(19) United States
(12) Patent Application Publication
(10) Pub. No.: US 2022/0133911 A1
(43) Pub. Date: May 5, 2022

(54) MYC, CYCLIN T1 AND/OR CDK9 FOR USE IN THE TREATMENT OF DEGENERATIVE HEART AND CNS DISORDERS

(71) Applicant: CAMBRIDGE ENTERPRISE LIMITED, Cambridge Cambridgeshire (GB)

(72) Inventors: Catherine Helen Wilson, Cambridge Cambridgeshire (GB); Megan Bywater, Queensland (AU)

(21) Appl. No.: 17/430,969

(22) PCT Filed: Feb. 14, 2020
(86) PCT No.: PCT/GB2020/050350

§ 371 Date: Aug. 13, 2021

(30) Foreign Application Priority Data
Feb. 14, 2019 (GB) 1902068.4

(51) Int. Cl. A61K 48/00; A61K 38/17; A61K 38/45; A61P 9/10

(52) U.S. Cl. CPC A61K 48/0066; A61P 9/10; A61K 38/45; A61K 38/1709

(57) ABSTRACT

The invention relates to expression of the transcription factor Myc and/or pTEF-b and their use as medicaments for inducing proliferation in cells with limited proliferative potential, such as cardiomyocytes. Also described are methods for the prevention and treatment of diseases, such as heart disease, associated with the loss of cells or cell death.

### FIELD OF THE INVENTION

The invention relates to expression of the transcription factor Myc and/or pTEF-b and their use as medicaments for inducing proliferation in cells with limited proliferative potential, such as cardiomyocytes. Also described are methods for the prevention and treatment of diseases, such as heart disease, associated with the loss of cells or cell death.

### BACKGROUND OF THE INVENTION

When an organ is damaged in order to maintain the integrity of the physiological and morphological state, the tissue must repair. The regenerative potential of an organ relates to the ability of the cells within that tissue to restore original tissue architecture post damage. Some organs have low regenerative potential and when damaged replace tissue with connective tissue that causes subsequent scarring. Certain organs, tissues and cells of the body are more capable of cellular proliferation (and hence regeneration) than others (Krafts, 2010). Broadly, organs can be separated into three categories based on their regenerative potential (Kotton and Morrisey, 2014). First, organs such as the intestine, skin and hematopoietic system that show high levels of cellular turnover and require a dedicated undifferentiated stem cell population to replace dead or sloughed-off cells. These tissues have enormous proliferative and self-renewing capacity. Second, quiescent tissues such as the liver, lung and pancreas respond with a proliferative burst after injury to replace lost cells (Kotton and Morrisey, 2014). Third, non-dividing tissues such as the central nervous system (CNS) and heart regenerate poorly or not at all after injury. These tissues are for the most part, incapable of self-repair and regeneration. Much research is devoted to developing technologies and techniques to aid or promote healing when organs are damaged.

There are ˜23 million heart failure patients worldwide, a condition for which there is currently no cure and current treatments only slow disease progression. The initiating cause of most instances of heart failure is a loss of cardiomyocytes, which are never replenished due to the low intrinsic regenerative capacity of the adult heart (Porrello et al., 2011; Porrello and Olson, 2014). Likewise, nervous system injuries affect over 90,000 people every year (Stabenfeldt et al., 2006). Traumatic injury to the CNS causes cell death and, for the most part, the CNS is incapable of regeneration of these lost cells. Control of neuron regeneration is both a major unmet medical need and an unsolved problem in neurobiology (Mahar and Cavalli, 2018). The failure of damaged adult CNS axons to regrow results in permanent disabilities for individuals with spinal cord injury or stroke (Mahar and Cavalli, 2018) which has enormous socio-economic impact. Furthermore, many diseases for example, multiple sclerosis, could be alleviated by regeneration of the CNS.

Regeneration of cells is essentially mediated via genetic regulation and the identification of factors that are capable of re-activating proliferation in adult tissue has become a promising avenue of research. While a variety of different factors can increase proliferation, the effects on regeneration have been modest (Leach et al., 2017; Mohamed et al., 2018; Nakada et al., 2017). Therefore, further understanding the cell intrinsic mechanisms of regeneration of these non-dividing cells is imperative if innate regenerative capacity is to be harnessed.

Myc is a basic helix-loop-helix-leucine zipper (bHLH-LZ) transcription factor that binds preferentially to specific sequences in the genome, termed E-boxes (Blackwood and Eisenman, 1991), via association with its bHLH-LZ heterodimerisation partner Max (Amati et al., 1993, 1992; Blackwood et al., 1992). Myc functions principally as a transcriptional activator by potentiating transcription initiation via association with various cofactors such as TRRAP and associated histone acetyl-transferases (Bouchard et al., 2001; McMahon et al., 1998) and facilitating productive transcriptional elongation by promoting RNA PolII loading and via its association with positive transcription elongation factor (P-TEFb) (De Pretis et al., 2017; Eberhardy and Farnham, 2002; Kanazawa et al., 2003; Rahl et al., 2010). P-TEFb is comprised of Cdk9 and Cyclin T1 which are stringently regulated by various transcriptional and post transcriptional mechanisms (Jonkers and Lis, 2015; Peterlin and Price, 2006; Zhou et al., 2012; Zhou and Yik, 2006), and dynamically controlled by an association with inactivation complex comprised of 7SK snRNA, Larp7, MEPCE and HEXIM (Barboric et al., 2005; He et al., 2008; Yik et al., 2003). P-TEFb phosphorylates Serine 2 of the C-terminal Domain (CTD) of paused RNA PolII leading to productive elongation (Jonkers and Lis, 2015; Peterlin and Price, 2006; Zhou et al., 2012; Zhou and Yik, 2006).

Myc is a highly pleiotropic transcription factor which coordinates multiple transcriptional programmes involved in cell replication and differentiation, metabolism and apoptosis (Amati et al., 1993; Dang, 2013; Eilers et al., 1991; Evan et al., 1992; Roussel et al., 1991). With the development of animal models allowing for switchable ectopic Myc expression in vivo, it has become evident that Myc also governs diverse cell extrinsic processes required for tissue regeneration—such as angiogenesis, modulation of the local inflammatory and immune responses, invasion and migration—but in a manner that is tightly tailored to the tissue in which Myc is activated (Kortlever et al., 2017; Shchors et al., 2006; Sodir et al., 2011). Since deregulated and elevated Myc expression is a pervasive and causal attribute of most, perhaps all, tumours, understanding how tissue-specific responses to Myc are determined at a molecular level is imperative.

Comprehensive analysis of Myc transcriptional output in individual cell types indicates that Myc regulates the expression of thousands of genes, perhaps as much as a third of the transcriptome. Such studies show great diversity across experimental platforms and hint that components of the transcriptional repertoire of Myc are highly context specific. In particular, genome-wide analysis of Myc occupancy indicates the presence of Myc on virtually all promoters with open chromatin, suggesting that tissue-specific variations in Myc activity result from specific, pre-existing resident cellular programmes. However, this large and diverse number of potential Myc target genes, and the lack of comparative analysis of transcriptional responses to Myc in different tissues, have together confounded reliable identification of common and tissue-specific Myc-dependent transcriptional programmes. Moreover, recruitment of Myc to a given gene does not always correlate with its level of transcription and binding efficiency and transcriptional outputs are influenced significantly by different levels of Myc expression. Nonetheless, it remains clear that Myc is a selective transcription factor that regulates a defined set of core genes across multiple tissues, and others that are tissue-specific

As discussed above, there is a need to understand the intrinsic cellular mechanisms of regeneration or lack of in non-dividing cells, and moreover, a need to be able to influence these mechanisms such that the regenerative potential of these cells can be re-established. The present invention addresses this need.

### SUMMARY OF THE INVENTION

Using a reversibly switchable mouse model in which supraphysiological levels of Myc are expressed comparably across different tissues we have determined that there are three general classes of response to activation of oncogenic levels of Myc: tissues that proliferated in response to the activation of ectopic Myc; tissues that did not; and tissues that exhibited innately high endogenous Myc and proliferative indices. The correlation between proliferative response to ectopic Myc in these tissues and the innate regenerative potential of each tissue is striking and suggests an underlying mechanistic connection. We have further determined that tissue regenerative capacity is tightly linked to the capacity of that tissue to respond to Myc and that tissue Myc responsiveness is governed principally by availability of key components of the core transcriptional machinery, namely p-TEFb, which Myc co-opts to drive its regenerative biological output. As such, we have determined that increasing the expression and/or activity of at least one, preferably both of Myc and p-TEFb, can increase or rather re-establish regenerative proliferation in adult tissues without proliferative potential. Where we refer to p-TEFb herein is meant cyclin T1 and/or cyclin-dependent kinase 9 (CDK9).

In one aspect of the invention there is provided a nucleic acid molecule comprising (at least one) nucleic acid sequence encoding at least one of a myc transcription factor, cyclin T1 and cyclin-dependent kinase 9 (CDK9) for use as a medicament.

In one embodiment, the nucleic acid is a ribonucleotide, preferably mRNA. In a further preferred embodiment, the mRNA molecule comprises at least one modification selected from a cap modification, a tail modification, a nucleoside modification or an untranslated region (UTR) modification.

In one embodiment, the mRNA molecule encodes a myc transcription factor as defined in SEQ ID NO: 11 or a functional variant thereof. In a further embodiment, the mRNA molecule encodes a cyclin T1 protein as defined in SEQ ID NO: 5 or a functional variant thereof. In another embodiment, the mRNA molecule encodes a CDK9 protein as defined in SEQ ID NO: 7 or a functional variant thereof.

In an alternative embodiment, the nucleic acid molecule is a nucleic acid construct or vector. In a preferred embodiment, the nucleic acid sequence encodes an inducible myc transcription factor operably linked to a first regulatory sequence.

In a further embodiment, the nucleic acid construct or vector further comprises a nucleic acid sequence encoding a cyclin T1 and/or a cyclin-dependent kinase 9 (CDK9) operably linked to the first regulatory sequence or a second regulatory sequence.

In one embodiment, the nucleic acid sequence encodes an inducible myc transcription factor as defined in SEQ ID NO: 1 or 10 or a functional variant thereof. In another embodiment, the nucleic acid sequence encodes a cyclin T1 protein as defined in SEQ ID NO: 5 or a functional variant thereof. In a further embodiment, the nucleic acid sequence encodes a CDK9 protein as defined in SEQ ID NO: 7 or a functional variant thereof.

In one embodiment, the regulatory sequence is a promoter. In one example, the promoter is the CAG promoter.

In one embodiment, the vector is a viral vector. In one preferred embodiment the viral vector is an adeno-associated viral vector.

In another aspect of the invention there is provided a composition comprising a mRNA molecule as described above.

In another aspect of the invention there is provided a composition comprising a first modified mRNA molecule and at least a second modified mRNA molecule, wherein the first modified mRNA molecule encodes a myc transcription factor and the second modified mRNA molecule encodes a cyclin T1 and/or cyclin-dependent kinase 9 (CDK9) protein, wherein the modification is selected from at least one of a cap modification, a tail modification, a nucleoside modification and a untranslated region (UTR) modification.

In a preferred embodiment, the composition comprises a first modified mRNA molecule, a second modified mRNA molecule and a third modified mRNA molecule, wherein the first modified mRNA molecule encodes a myc transcription factor, the second modified mRNA molecule encodes a cyclin T1 and the third modified mRNA molecule encodes a cyclin-dependent kinase 9 (CDK9) protein, wherein the modification is selected from at least one of a cap modification, a tail modification, a nucleoside modification and a untranslated region (UTR) modification.

In another aspect of the invention there is provided a composition comprising a first vector and at least a second vector, wherein the first vector comprises a nucleic acid sequence encoding an inducible myc transcription factor operably linked to a regulatory sequence and the second vector comprises a nucleic acid sequence encoding a cyclin T1 and/or a cyclin-dependent kinase 9 (CDK9) operably linked to a regulatory sequence.

In a further embodiment the composition comprises a first, second and third vector, wherein the first vector comprises a nucleic acid sequence encoding an inducible myc transcription factor operably linked to a regulatory sequence, the second vector comprises a nucleic acid sequence encoding a cyclin T1 operably linked to a regulatory sequence and the third vector comprises a nucleic acid sequence encoding a cyclin-dependent kinase 9 (CDK9) operably linked to a regulatory sequence.

In another aspect of the invention there is provided a composition as described herein for use as a medicament.

In a further aspect of the invention there is provided a nucleic acid molecule as described herein or a composition as described herein for use in the treatment of a condition characterised by the loss of cells or cell death.

In a preferred embodiment, the condition is selected from myocardial infarction, reduced ejection fraction of the heart, stroke, spinal cord injury or a neurodegenerative disorder.

In another aspect of the invention there is provided a method of therapy, the method comprising administering to an individual or patient in need thereof a nucleic acid molecule as described herein or a composition as described herein.

In one embodiment, the method is for the treatment of a condition characterised by cell loss. In a preferred embodiment, the condition is selected from myocardial infarction, reduced ejection fraction of the heart, stroke, spinal cord injury or a neurodegenerative disorder.

In another aspect of the invention there is provided a method of increasing at least one of cell proliferation, mitosis and cytokinesis in a cell, the method comprising introducing to the cell a nucleic acid molecule as described herein or a composition as described herein.

In a further aspect of the invention there is provided a method of increasing organ size, the method comprising introducing to the organ a nucleic acid molecule as described herein or a composition as described herein.

In another aspect of the invention, there is provided a host cell comprising the nucleic acid molecule as described herein or a composition as described herein. Preferably the host cell is a eukaryotic cell, preferably a mammalian cell and more preferably a heart, brain or kidney cell.

In a final aspect of the invention there is provided a nanoparticle comprising a nucleic acid molecule as described herein.

### DESCRIPTION OF THE FIGURES

The invention is further described in the following non-limiting figures:

FIG. 1 shows the proliferative response to supraphysiological Myc is very variable across different tissues.

(a) Immunoblot analysis of MycERT2 and endogenous c-Myc protein levels in wild-type (R26+/+) murine embryonic fibroblasts (MEFs) maintained in serum-deprived media, and at the indicated type points (in hours) post addition of serum, all compared with asynchronous R26+/+, R26MER/+, R26MER/MER, R26CMER/+, R26MER/CMER, R26CM ER/CM ER MEFs. Expression of Actin is included as a loading control.

(b) Immunohistochemical and immunofluorescence staining of Ki67 and BrdU in the brain, heart, kidney, lung, pancreas, liver, spleen and thymus isolated from wild-type (R26+/+) and R26CMER/+ mice 24 hours post administration of tamoxifen, Representative images based on analysis of 5 independent mice.

(c) Quantification of p-H3-positive nuclei percentage from brain, heart, kidney, lung, pancreas, liver (hepatocytes), spleen (red pulp) and thymus isolated from oil treated R26^{CMER/+} (n≥3) mice or wild-type (R26+/+, n≥3) and R26CMER/+ (n=3) mice 24 hours post administration of tamoxifen. Mean of 5 images per mouse; error bars, s.e.m.

(d) Immunoblot analysis of MycERT2 and endogenous c-Myc expression in the brain, heart, kidney, lung, pancreas, liver, spleen and thymus isolated from R26CMER/+ mice. Sample loading was normalized for equal protein content, as determined by a bicinchoninic acid assay (BCA). Expression of GAPDH is included as a confirmation of efficient protein isolation. Representative results based on analysis of 4 independent mice.

FIG. 2 shows Myc binding is dictated by chromatin access and differs between tissues. However, common Myc binding sites in the heart and liver are at genes involved in mitotic cell cycle and gene expression.

(a) The number of peaks called for c-Myc ChIP sequencing performed on chromatin isolated from hearts and livers harvested from wild-type (R26+/+) and R26CMER/+ mice 4 hours post administration of 4-OHT, and their location in relation to coding regions of the genome (promoter, intragenic, intergenic). Replicates are derived from independent mice.

(b) Venn diagram of the overlap of peaks called within promoter regions (−2 kb to +1 kb from the nearest TSS), identified by Myc ChIP sequencing on chromatin isolated from heart and livers harvested from R26CMER/+ mice (n=2) 4 hours post administration of 4-OHT.

(c) Venn diagram of the overlap of peaks called within distal elements, identified by Myc ChIP sequencing performed on the heart and livers isolated from R26CMER/+ mice (n=2) 4 hours post administration of 4-OHT.

(d) Common Myc-bound promoters in both the liver and heart. Motif probability curves show the probability of an E-box consensus sequence occurring at a given position relative to the Myc ChIP peak at each common promoter site, as determined by CentriMo (top left). Average read count of Myc peaks at common promoters containing 0, 1 or >2 E-box motifs within 1,000 bp from the peak centre in Liver and Heart chromatin (bottom left). Selected significant GO Biological Process Gene sets that overlap with common Myc-bound promoter elements (right).

(e) Liver-specific Myc-bound promoters bound by Myc only in the liver. Motif probability curves show the probability of an E-box consensus sequence occurring at a given position relative to the Myc ChIP peak at each liver-specific promoter site, as determined by CentriMo (left). Average read count of Myc peaks shown at common (black) and liver-specific (green) promoter sites (centre). Selected significant Mouse Gene Atlas Gene sets are shown that overlap with liver-specific Myc-bound promoter elements (right).

(f) Heart specific Myc-bound promoters bound by Myc only in the heart. Motif probability curves show the probability of an E-box consensus sequence occurring at a given position 5 relative to the Myc ChIP peak at each heart-specific promoter site, as determined by CentriMo (left). Average read count of Myc peaks shown at common (black) and heartspecific (red) promoter sites (centre). Significant Mouse Gene Atlas Gene sets are shown that overlap with heart-specific Myc-bound promoter elements (right).

(g) Heat map of peaks called by DNAse treated, acetylated H3K27 (H3K27ac), tri10 methylated H3K4 (H3K4me3) and RNA Polymerase II (PolII) ChIP sequencing at Mycbound promoter elements that are common between both the liver and heart (commonblack) or specific for an individual tissue (liver specific-green, heart specific-red). Myc ChIP sequencing was performed on the heart and livers isolated from R26CMER/+ mice 4 hours post administration of 4-OHT, overlap of n=2. PolII ChIP sequencing performed on the heart and livers isolated from wild-type (R26+/+ 15) mice, overlap of n=2. ChIP sequencing data for DNase treated, H3K27ac and H3K4me3 were taken from the ENCODE Project (accession numbers; GSM1014166, GSM1000093, GSM769017, GSM1014195, GSM1000140, GSM769014).

(h) As in (g) but for distal elements.

(i) Heat map of peaks called for ATAC seq, acetylated H3K27 (H3K27ac) and tri-methylated H3K4 (H3K4me3) ChIP sequencing on purified cardiomyocytes (CM) compared to peaks called by DNAse treated, acetylated H3K27 (H3K27ac), tri-methylated H3K4 (H3K4me3) and RNA Polymerase II (PolII) ChIP sequencing on whole heart (Heart) and liver (Liver) at Mitotic Cell Cycle genes (GO Biological process gene set 0000278) with Myc-bound promoter elements that are common between both the liver and heart (common-black) or specific for an individual tissue (liver specific-green, heart specific-red). Myc ChIP sequencing was performed on the heart and livers isolated from R26^{CMER/+} mice 4 hours post administration of 4-OHT, overlap of n=2. PolII ChIP sequencing performed on the heart and livers isolated from wild-type (R26+1 mice, overlap of n=2. ChIP sequencing data for DNase treated, H3K27ac and H3K4me3 were taken from the ENCODE Project (accession numbers; GSM1014166, GSM1000093, GSM769017, GSM 1014195, GSM 1000140, GSM769014), cardiomyocyte ATAC sequencing accession: GSE95763 (39), cardiomyocyte H3K27ac and H3K4me3 ChIP sequencing accession: SRP033385 (90).

FIG. 3 shows Myc-directed transcriptional targets differ between tissues.

(a) The number of differentially expressed genes in the heart, kidney, lung and liver of R26CMER/+ (n≥3) compared to wild-type (R26+/+, n≥3) mice 4 hours post administration of 4-OHT, as determined by RNA sequencing.

(b) Venn diagram depicting overlap of peaks within promoter regions identified by c-Myc ChIP sequencing performed on chromatin from heart (Heart MYC ChIP, red) or liver (Liver MYC ChIP, green) isolated from R26CMER/+ mice together with genes differentially expressed in response to supraphysiological Myc in these same tissues (UP DEGs-black, DOWN DEGs-grey), as determined by RNA sequencing on tissues isolated from R26CMER/+ versus wild-type (R26+/+) mice. All analysis performed at 4 hours post administration of 4-OHT. Areas are proportional to the numbers of promoters or differentially expressed genes within each cohort.

(c) Venn diagram depicting numbers of genes showing increased expression in response to supraphysiological Myc (UP DEGs) in the kidney (yellow), liver (green), heart (red) and lung (blue) of R26CMER/+ (n≥3) mice relative to wild-type (R26+/+, n≥3), as determined by RNA sequencing (FDR<0.05 and abs(log 2FC)>0.5) at 4 hours post administration of 4-OHT.

(d) 6 most significant GO Biological Process Gene sets that overlap with listed genes, showing a differential increase in expression (UP DEGs) in two or more tissues (kidney, liver, heart, lung) of R26CMER/+ (n≥13) mice relative to wild-type (R26+/+, n≥3), as determined by RNA sequencing at 4 hours post administration of 4-OHT.

(e) Venn diagram depicting numbers of genes showing decreased expression in response to supraphysiological Myc (DOWN DEGs) in the kidney (yellow), liver (green), heart (red) and lung (blue) of R26CMER/+ (n≥3) mice relative to wild-type (R26+/+, n≥3), as determined by RNA sequencing at 4 hours post administration of 4-OHT.

(f) 6 most significant GO Biological Process Gene sets that overlap with listed genes that show a differential decrease in expression (DOWN DEGs) in two or more tissues (kidney, liver, heart, lung) of R26CMER/+ (n≥3) mice relative to wild-type (R26+/+, n≥3), as determined by RNA sequencing at 4 hours post administration of 4-OHT.

(g) 3 most significant Mouse Gene Atlas Gene sets that overlap with listed genes that show a differential decrease in expression (DOWN DEGs) only in the liver (liver-specific) of R26CMER/+ (n≥3) mice relative to wild-type (R26+/+, n≥3), as determined by RNA sequencing at 4 hours post administration of 4-OHT.

(h) 6 most significant GO Biological Process Gene sets that overlap with listed genes that show a differential decrease in expression (DOWN DEGs) only in the liver (liver-specific, absent in kidney, heart and lung) of R26CMER/+ (n≥3) mice relative to wild-type (R26+/+, n≥3), as determined by RNA sequencing at 4 hours post administration of 4-OHT.

(i) Heatmap showing the union of DEGs called in each tissue; liver, lung, kidney and heart. Shown are mRNA expression fold changes (Log 2) upon MycERT2 activation relative to wild-type as determined by RNA sequencing of R26CMER/+ (n≥3) mice relative to wild-type (R26+/+, n≥3) at 4 hours post administration of 4-OHT.

(j) Hearts isolated from R26^{CMER/+} mice show enrichment of common Myc targets (exclusive to the liver, lung and kidney) in comparison to wild-type (R26^{+/+}), as determined by RNA sequencing at 4 hours post administration of 4-OHT.

FIG. 4 shows the heart has low levels of endogenous P-TEFb

(a) Immunoblot analysis of the C-terminal domain of RNA Polymerase II—total (Rpb1) and phosphorylated (p-Rpb1(S5) and p-Rpb1(S2)), CDK9, Cyclin T1 and Larp7 expression in the heart, liver, lung and kidney isolated from wild-type (R26+/+) mice. Sample loading was normalized for equal protein content. Expression of GAPDH is included as a confirmation of efficiency of protein isolation and comparable loading between individual tissue samples. Replicate samples are derived from independent mice. The composite figure is generated from the same samples loaded across multiple blots and a representative image for GAPDH is shown.

(b) Immunoblot analysis of NELF-A, NELF-B, NELF-C/D, NELF-E, SPT4 and SPT5 expression in the heart, liver, lung and kidney isolated from wild-type (R26+/+) mice. Sample loading was normalized for equal protein content. Expression of GAPDH is included as a confirmation of efficiency of protein isolation and comparable loading between individual tissue samples. Replicate samples are derived from independent mice. The composite figure is generated from the same samples loaded across multiple blots and a representative image for GAPDH is shown.

(c) Quantitative RT-PCR analysis of 7SK snRNA in liver and heart isolated from wild-type mice (n=5). Expression is normalized to Actin and Gapdh and relative to liver. Error bars show s.e.m. Unpaired t-test; liver vs heart P=0.019*. Replicate samples are derived from independent mice.

FIG. 5 shows that overexpression of Ccnt1 in vitro increases efficiency of Myc driven transcription in cardiomyocytes.

(a) Quantitative RT-PCR analysis of Myh6 in the heart (n=4), kidney (n=4), lung (n=4), pancreas (n=4), liver (n=4), spleen (n=4) and thymus (n=3) of wild-type mice, and cardiomyoctyes (n=3) isolated from wild-type mice. Expression is normalized to Actin and Gapdh and relative to liver. Error bars show s.e.m. Replicate samples are derived from independent mice.

(b) Phase contrast images of adult cardiomyocytes in culture 48 hours post infection and accompanying fluorescent images to determine transfection efficiency on the basis of fluorescent protein marker expression, either GFP (Ad-CMV-GFP) (left) or RFP (Ad-AMV-Ccnt1-RFP) (right). Scale bar=400 μm.

(c) Immunoblot analysis of Cyclin T1 and phosphorylated C-terminal domain of RNA Polymerase II (p-Rpb1(S2)) in R26CMER/+ primary cardiomyocytes infected with an adenovirus encoding either GFP (Ad-GFP) or Ccnt1 (Ad-Ccnt1). Replicate samples are derived from independent primary cardiomyocyte isolations.

(d) Quantitative RT-PCR analysis of Cad, Bzw2, Pinx1, Polr3d, St6 and Cdc25a in wild type (R26+/+, n≥4) and R26CMER/+ (n≥4) primary cardiomyocytes infected with an adenovirus encoding either GFP (Ad-GFP) or Ccnt1 (Ad-Ccnt1), 4 hours post addition of 100 nM 4-OHT. Expression is normalized to Gapdh and Actin and relative to an individual wild-type (R26+/+) Ad-GFP control. Error bars show s.d. Two Way ANOVA with Tukey's multiple comparisons test; Ad-GFP R26+/+vs R26CMER/+: P=0.05* (Cad), Ad-Ccnt1 R26+/+vs R26CMER/+: P=0.05* (Cad, Pinx1, Polr3d, St6) P=0.01** (Bzw2, Cdc25a). Replicate samples are derived from independent primary cardiomyocyte isolations.

FIG. 6 shows that the juvenile heart has high levels of P-TEFb and is permissive for Myc-driven transcriptional activation

(a) Immunoblot analysis of the C-terminal domain of RNA Polymerase II, total (Rpb1) and phosphorylated (p-Rpb1(S2)), Cyclin T1 and CDK9 protein expression in wild-type heart and liver isolated from 15 day-old (15 d) and 60 day-old (60 d) mice. Expression of GAPDH is included as a loading control. Replicate samples are derived from independent mice.

(b) Quantitative RT-PCR analysis of Cad, Bzw2, Pinx1, Polr3d, St6 and Cdc25a expression in wild-type (R26+/+, n≥6) and R26CMER/+ (n≥6) heart isolated from 15 day-old (15 d) mice versus heart and liver isolated from 60 day-old (60 d) mice 4 hours post administration of 4-OHT. Expression is normalized to Actin and Gapdh and relative to the respective wildtype (R26+/+). Error bars show s.d. Two Way ANOVA with Tukey's multiple comparisons test; 15 day R26+/+4-OHT vs R26CMER/+4-OHT: P=0.001*** (Cad, Bzw2, Pinx1, Polr3d, St6 and Cdc25a), 15 day R26+/+4-OHT vs R26CMER/+4-OHT: P=0.001** (Polr3d), adult heart R26+/+4-OHT vs R26CMER/+4-OHT: P=0.001*** (Cad), adult liver R26+/+4-OHT vs R26CMER/+4-OHT: P=0.001*** (Cad, Bzw2, Pinx1, Polr3d, St6 and Cdc25a). Replicate samples are derived from independent mice.

(c) Heatmap showing the union of DEGs called in each tissue; adult liver, adult heart and P15 heart. mRNA expression fold changes (Log 2) upon MycER activation are shown relative to wild type, as determined by RNA sequencing of R26CMER/+ (n≥3) tissues relative to wild-type (R26+/+, n≥3) at 4 hours post administration of 4-OHT.

(d) Quantification of p-H3 positive nuclei percentage in heart (positive cardiomyocyte nuclei only) and liver (positive hepatocyte nuclei only) isolated from 15 day-old (15d) and 60 day-old (60d) wild-type (R26+/+, n≥7) and R26CMER/+ (n≥4) mice 24 hours post administration of tamoxifen. Means are taken from 5 images per mouse; Error bars show s.d. Two Way ANOVA with Tukey's multiple comparisons test; R26+/+ 4-OHT vs R26CMER/+ 4-OHT: P=0.001*** (15 day heart and adult liver).

(e) Immunofluorescent staining of cardiac troponin, p-H3, Ki67, PCM1, Wheat Germ Agglutinin and Aurora B positive mid-body in the heart of 15 day-old control (R26^{LSL-CMER/+}; no cre) and Myh6-Cre; R26^{LSL-CMER/+} (R26^{LSL-CMER/+}; cre+) mice 48 hours post administration of tamoxifen. Representative images based on analysis of 5 independent mice.

(f) Image of the whole heart and a tibia isolated from a 15 day-old control (R26^{LSL-CMER/+}; no cre) and Myh6-Cre; R26^{LSL-CMER/+} (R26^{LSL-CMER/+}; cre+) mice 48 hours post administration of tamoxifen. Scale bar represents 10 mm. Representative images based on analysis on >4 independent mice.

(g) The weight (mg) of hearts (left) and quantification of the number of cardiomyocytes (right) isolated from 15 day-old control (R26^{LSL-CMER/+}; no cre, n=4) and Myh6-Cre; R26^{LSL-CMER/+} (R26^{LSL-CMER/+}; cre+, n=7) mice 48 hours post administration of tamoxifen or oil (veh), expressed as fold change over the length (mm) of a tibia isolated from the same mouse. Error bars show s.e.m. Unpaired t-test; no cre vs cre+P=0.0003*** (weight), Mann Whitney test; no cre vs cre+ P=0.0061** (cardiomyoctyte number). Replicate samples are derived from independent mice.

(h) Immunofluorescent staining of cardiac troponin T (green), p-H3 (red) and DNA (blue) on cardiomyocytes isolated from fixed and dissociated hearts of 15 day-old control (R26^{LSL-CMER/+}; no cre) and Myh6-Cre; R26^{LSL-CMER/+} (R26^{LSL-CMER/+}; cr+) mice

(i) Three dimensional render (second from left) generated from 500 image z-stack (50 μm) of transmission electron micrographs from a Myh6-Cre; R26^{LSL-CMER/+} heart 48 hours post administration of tamoxifen. Green shows outline of cells, blue shows nuclei, pink line shows site of cross section through cells, outlined in green (second from right), and expanded (right). Left image shows mitotic nuclei, arrow. Structures are indicated; f-myofibrils, n-nucleus, m-mitochondria, bv-blood vessel.

(j) Immunoblot analysis of the C-terminal domain of RNA Polymerase II, total (Rpb1) and phosphorylated (p-Rpb1(S2)), MycERT2, Cyclin T1 and phosphorylated ERK1 (T202/T204) and ERK2 (T185/T187) protein expression in hearts isolated from control (R26+/+; TetO-HRas, R26+/+; Myh6-tTA or R26+/+), TetO-HRas; Myh6-tTA; R26+/+ (R26+/+;HRas), R26CMER/+ and TetO-HRas; Myh6-tTA; R26 CMER/+ (R26 CMER/+;HRas) mice 4 weeks post withdrawal of doxycycline and 4 hours post administration of 4-OHT. Replicate samples are derived from independent mice.

(k) Venn diagram of the number of genes showing an increase in expression in response to supraphysiological Myc (UP DEGs) in the kidney (yellow), liver (green) and lung (blue) of R26CMER/+ (R26 CMER/+, n≥3) in comparison to wild-type (R26+/+, n≥3) and heart (red) from TetO-HRas; Myh6-tTA; R26 CMER/+ (R26 CMER/+;HRas) mice (n=3) in comparison to R26+/+; TetO-HRas; Myh6-tTA mice (n=3) four weeks post withdrawal of doxycycline, as determined by RNA sequencing (FDR<0.05 and abs(log 2FC)>0.5) at 4 hours post administration of 4-OHT.

FIG. 7 shows that overexpression of Ccnt1 in the adult heart leads to Myc driven transcription and proliferation of cardiomyocytes

(a) Immunohistochemistry (left) and quantification (right) of Ccnt1 in the liver isolated from adult R26^{LSL-CMER/+} or heart isolated from Myh6-Cre; R26^{LSL-CMER/+} mice systemically infected with adeno associated virus (serotype 9, 1×1011 vg/mouse) encoding either RFP (AAV9-RFP, n=5) or Ccnt1 (AAV9-Ccnt1, n=4) 4 weeks post infection. Representative images based on analysis of 5 independent mice.

(b) Immunoblot analysis of the C-terminal domain of phosphorylated RNA Polymerase II p-Rpb1(S2)), CDK9 and Cyclin T1 expression in the heart isolated from adult Myh6-Cre; R26^{LSL-CMER/+} mice systemically infected with adeno associated virus encoding either RFP (AAV9-RFP) or Ccnt1 (AAV9-Ccnt1) 4 weeks post infection. Sample loading was normalized for equal protein content. Expression of GAPDH is included as a confirmation of efficiency of protein isolation and comparable loading between individual tissue samples. Replicate samples are derived from independent mice.

(c) Quantitative RT-PCR analysis of Cad, Bzw2, Pinx1, Polr3d, St6 and Cdc25a expression in control (R26^{LSL-CMER/+}, no cre, n=5) and Myh6-Cre; R26^{LSL-CMER/+} (R26^{LSL-CMER/+}; cre+, n=7) adult mouse heart isolated 4 weeks post systemic infection with an adeno associated virus encoding Ccnt1 (AAV9-Ccnt1) and 4 hours post administration of 4-OHT. Expression is normalized to Actin and Gapdh and relative to the respective wildtype (R26^{LSL-CMER/+}). Error bars show s.e.m. Unpaired t-test; no cre vs cre+: P<0.0005***(Cad, Bzw2, Pinx1 and St6), P=0.0013** (Cdc25a), P=0.0035** (Polr3d). Replicate samples are derived from independent mice.

(d) Enrichment of common Myc targets (liver, lung, kidney and heart) in cardiomyocytes isolated from the heart of AAV-CCNT1 infected Myh6-cre; R26^{LSL-CMER/+} (Myc+CCNT1) mice in comparison to uninfected Myh6-cre; R26^{LSL-CMER/+} mice (Myc) at 4 hours post administration of 4-OHT, as determined by RNA sequencing. Including normalised enrichment score (NES) and FDR q-value (FDR).

(e) Immunofluorescent staining of cardiac troponin, p-H3,Ki67, PCM1, Wheat Germ Agglutinin (WGA) and Aurora B in control (R26^{LSL-CMER/+}, no cre) and Myh6-Cre; R26^{LSL-CMER/+} (R26^{LSL-CMER/+}, cre+) adult mouse heart isolated 4 weeks post systemic infection with an adeno associated virus encoding Ccnt1 (AAV9-Ccnt1) and 48 hours post administration of tamoxifen. Representative images based on analysis of 5 independent mice.

(f) Quantification of p-H3 positive nuclei percentage in control (R26^{LSL-CMER/+;} no cre, n=9) and Myh6-Cre; R26^{LSL-CMER/+} (R26^{LSL-CMER/+},cre+, n=7) adult mouse heart isolated 4 weeks post systemic infection with an adeno associated virus encoding Ccnt1 (AAV9-Ccnt1) and 48 hours post administration of tamoxifen. Means are taken from 5 images per mouse; Error bars show s.e.m. Unpaired t-test; no cre vs cre+P<0.0001.

(g) The weight (mg) of hearts isolated from control (R26^{LSL-CMER/+;} no cre, n>5) and Myh6-Cre; R26^{LSL-CMER/+} (R26^{LSL-CMER/+}, n>6) adult mouse heart 4 weeks post systemic infection with an adeno associated virus encoding Ccnt1 (AAV9-Ccnt1) and 48 and 72 hours post administration of tamoxifen, expressed as fold change over the length (mm) of a tibia isolated from the same mouse. Error bars show s.e.m. Two Way ANOVA with Tukey's multiple comparisons test; no cre vs cre+: P=0.05* (48 hours), P=0.001*** (72 hours). Replicate samples are derived from independent mice.

(h) Image of the whole heart and a tibia from control (R26^{LSL-CMER/+}, no cre) and Myh6-Cre; R26^{LSL-CMER/+} (R26^{LSL-CMER/+}; cre+) adult mouse heart isolated 4 weeks post systemic infection with an adeno associated virus encoding Ccnt1 (AAV9-Ccnt1) and 72 hours post administration of tamoxifen. Scale bar represents 10 mm. Representative images based on analysis on independent mice.

(i) Quantification of the number and size of cardiomyocytes from control (R26^{LSL-CMER/+}, no cre, n=5) and Myh6-Cre; R26^{LSL-CMER/+} (R26^{LSL-CMER/+}, n=6) adult mouse heart isolated 4 weeks post systemic infection with an adeno associated virus encoding Ccnt1 (AAV9-Ccnt1) and 72 hours post administration of tamoxifen. Error bars show s.d. Mann Whitney test; no cre vs cre+P=0.0043**. Replicate samples are derived from independent mice.

FIG. 8 shows (a) Southern blot analysis of digested DNA from R26+/+ wild-type and R26CMER/+ mice, analysed with 5′ (EcoRI), 3′ (EcoRI/SalI) and internal (BamHI) probes. Wild-type fragments (11.5 kb, 5′; 10.3 kb, 3′ and 2.4 kb, internal) are present in wild-type and R26CMER/+ mice whereas the targeted fragments (2.3 kb, 5′; 9.5 kb, 3′ and 3.3 kb, internal) are present in only R26CMER/+ mice.

(b) Quantitative RT-PCR analysis of MycER in R26MER/+ (n=4) and R26CMER/+(n=4) liver, lung, pancreas, spleen, thymus, heart and kidney. Expression is normalized to Actin and Gapdh and relative to the respective R26MER/+. Error bars show s.e.m. Replicate samples are derived from independent mice.

(c) Immunoblot analysis of MycERT2 expression in the brain, heart, kidney, lung, pancreas, liver, spleen and thymus isolated from R26CMER/+ and R26CMER/CMER mice. Sample loading was normalized for equal protein content, as determined by a bicinchoninic acid assay (BCA). Expression of GAPDH is included as a confirmation of efficient protein isolation and equal loading within individual tissue samples.

(d) Quantitative RT-PCR analysis of Tomato in R26CMER/mTmG liver, lung, pancreas, spleen, thymus, heart and kidney isolated 4 hours post administration of oil (vehicle, n≥3) or tamoxifen (n≥3). Expression is normalized to Actin and Gapdh and relative to the respective vehicle control. Error bars show s.e.m. Replicate samples are derived from independent mice.

(e) Immunohistochemistry of Ki67 in the heart, kidney, lung and liver isolated from wild-type (R26+/+) and R26CMER/+ mice 72 hours post administration of tamoxifen. Representative images based on analysis of 4 independent mice.

(f) Quantification of p-H3 positive nuclei per field of view of the heart, kidney, lung and liver (positive hepatocyte nuclei only) isolated from wild-type (R26+/+, n=4) and R26CMER/+ (n=5) mice 72 hours post administration of tamoxifen. Means are taken from 5 images per mouse; error bars show s.d.

(g) Immunohistochemical staining of Ki67 in the heart, kidney, lung and liver isolated from wild-type (R26+/+), R26CMER/+ and R26CMER/CMER mice 24 hours post administration of tamoxifen Representative images based on analysis of at least 3 independent mice.

(h) Quantification of p-H3 positive nuclei per field of view of the heart, kidney, lung and liver (positive hepatocyte nuclei only) isolated from wild-type (R26+/+, n=3), R26CMER/+ (n=5) and R26CMER/CMER (n=4) mice 24 hours post administration of tamoxifen. Means are taken from 5 images per mouse; error bars show s.d.

FIG. 9 shows (a) Immunoblot analysis of the C-terminal domain of RNA Polymerase II, total (Rpb1) and c-Myc protein expression in wild-type heart isolated from embryos at 16.5 days post fertilisation (E16.5), neonates at birth (OD), 5 day (5D), 10 day (10D) 15 day (15D) and 20 day (20D) old mice and wild-type liver isolated from 60 day (60D) old mice. Expression of GAPDH is included as a loading control. Replicate samples are derived from independent mice.

(b) Longer exposure of Immunoblot shown in FIG. 9a, showing the C-terminal domain of RNA Polymerase II and total (Rpb1) and phosphorylated (p-Rpb1(S2)) expression in wild-type heart and liver isolated from 15 day-old (15d) and 60 day-old (60d) mice.

(c) Enrichment of common Myc targets (liver, lung, kidney and heart) in heart isolated from 15 day R26CMER/+ mice in comparison to controls (R26+/+) at 4 hours post administration of 4-OHT, as determined by RNA sequencing. Including normalised enrichment score (NES) and FDR q-value (FDR).

(d) Quantitative RT-PCR analysis of Cyclin D1, Cdk4, Cyclin B1 and Cdk1 in wild-type (R26+/+, n≥5) and R26CMER/+ heart isolated from 15 day-old (15d Heart, n≥4) mice versus heart and liver isolated from 60 day-old (60d Heart, n≥5) (60d Liver, n≥4) mice 4 (light grey) or 24 (dark grey) hours post i.p. of 4-OHT. Expression is normalized to Actin and Gapdh and relative to the respective wildtype (R26+/+). Error bars show s.d. One Way ANOVA with Tukey's multiple comparisons test; 15d heart R26+/+vs 24 hour R26CMER/+: P=0.001*** (Cdk4, Cyclin B1 and Cdk1), 60d heart R26+/+vs 24 hour R26CMER/+: P=0.01** (Cyclin D1) P=0.001*** (Cdk4), 60d liver R26+/+vs 4 hour R26CMER/+: P=0.05* (Cdk4), 60d liver R26+/+vs 24 hour R26CMER/+: P=0.01** (Cdk4) P=0.001*** (Cyclin D1, Cyclin B1, Cdk1). Replicate samples are derived from independent mice.

(e) Enrichment of pro-cell cycle progression gene sets in heart isolated from 15 day R26CMER/+ mice in comparison to controls (R26+/+) at 4 hours post administration of 4− OHT, as determined by RNA sequencing. Including normalised enrichment score (NES) and FDR q-value (FDR). Immunofluorescent staining of cardiac troponin and Aurora B in the heart of 15 day-old

(f) Myh6-Cre; R26LSL-CMER/+ mice 48 hours post administration of tamoxifen. Arrows show differences in Aurora B localization throughout the cell cycle. 1—G2, nucleus. 2—Metaphase, metaphase chromosomes. 3—Anaphase, mid-zone. 4—Cytokinesis, centrally located mid-body. 5—Failing cytokinesis—laterally displaced mid-body.

(g) Immunofluorescent staining of cardiac troponin T (green) positive cardiomyocytes isolated from fixed and dissociated hearts of 15 day-old control (R26LSL-CMER/+; no cre) and Myh6-Cre; R26LSL-CMER/+ (R26LSL-CMER/+;cre+) mice.

(h) Quantification of the length and width of cardiomyocytes isolated from the hearts of 15 day-old control (R26LSL-CMER/+; no cre, n=4) and Myh6-Cre; R26LSL-CMER/+ (R26LSL-CMER/+;cre+, n=6) mice 48 hours post administration of tamoxifen. Mean determined from the measurement of >13 individual cardiomyocytes per mouse. Error bars show s.e.m. Mann Whitney test; no cre vs cre+P=not significant. Replicate samples are derived from independent mice.

(i) Top-H&E staining, middle-WGA and p-H3 immunofluorescent staining, bottom-cardiac troponin immunofluorescent staining of hearts from 15 day-old control (R26LSL-CMER/+; no cre) and Myh6-Cre; R26LSL-CMER/+ (R26LSL-CMER/+;cre+) mice 96 hours post administration of tamoxifen.

FIG. 10 shows (a) Quantitative RT-PCR analysis of Cad, Bzw2, Pinx1, Polr3d, St6 and Cdc25a expression in adult wildtype (R26+/+, n=6) and R26CMER/+ mouse heart isolated 4 weeks post systemic infection with an adeno associated virus encoding either RFP (AAV9-RFP, n=4) or Ccnt1 (AAV9-Ccnt1, n=6) and 4 hours post administration of 4-OHT. Expression is normalized to Actin and Gapdh and relative to the respective wildtype. Error bars show s.e.m. Two Way ANOVA with Tukey's multiple comparisons test; R26+/+ AAV9-Ccnt1 vs R26CMER/+ AAV9-RFP: P=0.05* (Cad). R26+/+ AAV9-Ccnt1 vs R26CMER/+ AAV9-Ccnt1: P=0.01** (Bzw2, Cdc25a and Pinx1). R26+/+ AAV9-Ccnt1 vs R26CMER/+ AAV9-Ccnt1: P=0.001*** (Cad and St6).Replicate samples are derived from independent mice.

(b) Top-enrichment of common Myc targets (liver, lung, kidney and heart), bottom-enrichment of a pro-cell cycle progression gene set in heart isolated from adult Myh6-Cre; R26LSL-CMER/+ (cre+) in comparison to control (R26LSL-CMER/+, no cre), both treated with AAV-CCNT1 4 weeks before administration of 4-OHT (4 hr), as determined by RNA sequencing. Including normalised enrichment score (NES) and FDR q-value (FDR).

(c) Relative amounts of premature and mature mRNA expressed from common Myc target genes (left) and heart-specific genes (right) was determined from RNA sequencing performed on cardiomyocytes purified from adult Myh6-Cre; R26LSL-CMER/+ (cre+) and control (R26LSL-CMER/+, no cre) mice, both treated with AAV-CCNT1 4 weeks before administration of 4-OHT (4 hr), as determined by RNA sequencing. Intercept (I) and slope (S) included.

(d) Quantification of p-H3 positive nuclei per field of view from adult R26CMER/+ mouse hearts isolated 4 weeks post systemic infection with an adeno associated virus encoding either RFP (AAV9-RFP, n=5) or Ccnt1 (AAV9-Ccnt1, n=7) and 48 hours post administration of tamoxifen. Means are taken from 5 images per mouse; Error bars show s.e.m. Unpaired t-test; AAV9-RFP vs AAV9-Ccnt1, P=0.0110.

(e) Immunofluorescent staining of cardiac troponin (red), p-H3 (green, left) and Aurora B positive mitotic nuclei (green, right) from adult R26CMER/+ mouse hearts isolated 4 weeks post systemic infection with an adeno associated virus encoding either RFP (AAV9-RFP) or Ccnt1 (AAV9-Ccnt1) and 48 hours post administration of tamoxifen. Representative images based on analysis of 5 independent mice.

(f) Enrichment of mitosis and cytokinesis gene sets in heart isolated from adult Myh6-Cre; R26LSL-CMER/+ mice 4 weeks post systemic infection with AAV-CCNT1 verses AAV-RFP and hours post administration of tamoxifen, as determined by RNA sequencing. Including normalised enrichment score (NES) and FDR q-value (FDR).

(g) The weight (mg) of hearts isolated from adult R26CMER/+ mouse hearts isolated 4 weeks post systemic infection with an adeno associated virus encoding either RFP (AAV9-RFP, n=5) or Ccnt1 (AAV9-Ccnt1, n=15) and 48 hours post administration of tamoxifen, expressed as fold change over the length (mm) of a tibia isolated from the same mouse. Error bars show s.e.m. Unpaired t-test; AAV9-RFP vs AAV9-Ccnt1, P=0.0006. Replicate samples are derived from independent mice.

(h) Nuclei quantification performed on images of cardiomyocytes isolated from control (R26LSL-CMER/+; no cre) and Myh6-Cre; R26LSL-CMER/+ (R26LSL-CMER/+; cre+) adult mouse hearts isolated 4 weeks post systemic infection with an adeno associated virus encoding Ccnt1 (AAV9-Ccnt1) or RFP (AAV9-RFP) and 72 hours post administration of tamoxifen. Error bars show s.d. Mann Whitney test; no cre vs cre+P=0.0043**. Replicate samples are derived from independent mice.

Left—H&E staining, middle—IdU (BrdU) and WGA immunofluorescent staining and, right—Gomori Trichrome staining from control (R26LSL-CMER/+; no cre) and Myh6-Cre; R26LSL-CMER/+ (R26LSL-CMER/+; cre+) adult mouse heart. Mice were systemically infected with an adeno associated virus encoding Ccnt1 (AAV9-Ccnt1) at 4 weeks of age, given a single injection of tamoxifen to transiently activate MycER at 8 weeks of age and collected at 16 weeks of age.

FIG. 11 shows indirectly increasing PTEF-b activity leads to Myc driven transcription and proliferation in cardiomyocytes

(a) Quantification of p-H3 positive nuclei per field of view in hearts isolated from control (R26+/+; TetO-HRas, R26+/+; Myh6-tTA or R26+/+, black, n=8), TetO-HRas; Myh6-tTA; R26+/+ (R26+/+;HRas, red, n=5), R26CMER/+ (blue, n=4) and TetO-HRas; Myh6-tTA; R26 CMER/+ (R26 CMER/+;HRas, purple, n=3) mice 4 weeks post withdrawal of doxycycline and 24 hours post administration of tamoxifen. Representative images based on analysis of 5 images per mouse; error bars show s.e.m. One Way ANOVA with Tukey's multiple comparisons test; control vs TetO-HRas; Myh6-tTA; R26 CMER/+: P=0.001***.

(b) Immunohistochemistry of Ki67 in hearts isolated from control (R26+/+; TetO-HRas, R26+/+; Myh6-tTA or R26+/+), TetO-HRas; Myh6-tTA; R26+/+ (R26+/+;HRas), R26CMER/+ and TetO-HRas; Myh6-tTA; R26 CMER/+ (R26 CMER/+;HRas) mice 4 weeks post withdrawal of doxycycline and 24 hours post administration of tamoxifen. Representative images based on analysis of at least 3 independent mice.

(c) Immunofluorescent staining of cardiac troponin (red) and p-H3 positive mitotic nuclei (green) in the heart of TetO-HRas; Myh6-tTA; R26 CMER/+/+ (R26 CMER/+;HRas) mice and TetO-HRas; Myh6-tTA; R26+/+ control (R26+/+;HRas) 4 weeks post withdrawal of doxycycline and 24 hours post administration of tamoxifen. Representative images based on analysis of 5 independent mice.

(d) Immunofluorescent staining of cardiac troponin (red) and Aurora B (green) in the heart of TetO-HRas; Myh6-tTA; R26 CMER/+ (R26 CMER/+;HRas) mice and TetO-HRas; Myh6-tTA; R26+/+ control (R26+/+;HRas) 4 weeks post withdrawal of doxycycline and 24 hours post administration of tamoxifen. Representative images based on analysis of 5 independent mice.

(e) Quantitative RT-PCR analysis of Cyclin D1, Cdk4, Cyclin B1 and Cdk1 in control (C; R26+/+; TetO-HRas, R26+/+; Myh6-tTA or R26+/+, n≥24), TetO-HRas; Myh6-tTA; R26+/+ (R26+/+; HRas, n≥5), R26CMER/+ (n≥14) and TetO-HRas; Myh6-tTA; R26 CMER/+ (R26 CM ER/+;HRas, n=8) mice 4 weeks post withdrawal of doxycycline and 4 hours or 24 hours post administration of 4-OHT. Expression is normalized to Actin and Gapdh and relative to the respective control. Error bars show s.e.m. One Way ANOVA with Tukey's multiple comparisons test; control vs 24 hour R26CMER/+: P=0.05* (Cdk4), control vs 24 hour TetO-HRas; Myh6-tTA; R26 CMER/+: P***=0.001 (Cyclin D1, Cdk4, Cyclin B1, Cdk1). Replicate samples are derived from independent mice.

(f) Quantitative RT-PCR analysis of Cad, Bzw2, Pinx1, Polr3d, St6 and Cdc25a in control (R26+/+; TetO-HRas, R26+/+; Myh6-tTA or R26+/+, n≥14), TetO-HRas; Myh6-tTA; R26+/+ (R26+/+;HRas, n=4), R26CMER/+ (n=6) and TetO-HRas; Myh6-tTA; R26 CMER/+ (R26 CMER/+;HRas, n=5) mice 4 weeks post withdrawal of doxycycline and 4 hours post administration of 4-OHT. Expression is normalized to Actin and Gapdh and relative to the respective control. Error bars show s.e.m. Two Way ANOVA with Tukey's multiple comparisons test; control vs R26CMER/+: P=0.001*** (Cad), control vs TetO-HRas; Myh6-tTA; R26 CMER/+: P**=0.01 (Bzw2, Pinx1, Polr3d, Cdc25a) P***=0.001 (St6). Replicate samples are derived from independent mice.

(g) Heat map showing the union of DEGs called in each tissue; adult liver and adult heart. Shown are mRNA expression fold changes (Log 2) upon MycERT2 activation relative to wild-type, as determined by RNA sequencing of R26CMER/+ (n≥3) mice in comparison to wild-type (R26+/+, n≥3) and TetO-HRas; Myh6-tTA; R26 CMER/+ mice (R26 CMER/+;HRas, n=3) in comparison to TetO-HRas; Myh6-tTA; R26+/+ mice (R26+/+;HRas, n=3) at 4 hours post administration of 4-OHT.

(h) Heat map depicts gene expression of the “regeneration network” (described in Quaife-Ryan et al., 2017) from RNA-seq of TetO-HRas; Myh6-tTA; R26+/+(R26+/+;HRas, n=3) and TetO-HRas; Myh6-tTA; R26 CMER/+P60 adult hearts (R26 CMER/+;HRas, n=3). MIP1Myo and MIP56.Myo denote neonatal (P1) or adult (P56) cardiomyocytes isolated from myocardial infarction-operated hearts (n=4). Regeneration network genes were filtered for genes differentially expressed (DEGs) between TetO-HRas; Myh6-tTA; R26+/+ (R26+/+;HRas) and TetO-HRas; Myh6-tTA; R26 CMER/+(R26 CM ER/+;HRas) hearts (FDR<0.05).

(i) Immunoblot analysis of the C-terminal domain of RNA Polymerase II, total (Rpb1) and phosphorylated (p-Rpb1(S2)), MycERT2, Cyclin T1 and phosphorylated ERK1 (T202/T204) and ERK2 (T185/T187) protein expression in hearts isolated from control (R26+/+; TetO-HRas, R26+/+; Myh6-tTA or R26+/+), TetO-HRas; Myh6-tTA; R26+/+(R26+/+;HRas), R26CMER/+ and TetO-HRas; Myh6-tTA; R26 CMER/+ (R26 CMER/+;HRas) mice 4 weeks post withdrawal of doxycycline and 4 hours post administration of 4-OHT. Replicate samples are derived from independent mice.

FIG. 12 shows (a) Immunoblot analysis of the C-terminal domain of RNA Polymerase II—total (Rpb1) and phosphorylated (p-Rpb1(S2)), CDK9, Cyclin T1 and endogenous c-Myc in wild-type brain and kidney over time, compared to adult liver. Sample loading was normalized for equal protein content. Expression of GAPDH is included as a confirmation of efficiency of protein isolation and comparable loading between individual tissue samples. Replicate samples are derived from independent mice. The composite figure is generated from the same samples loaded across multiple blots and a representative image for GAPDH is shown. (b) Immunohistochemical staining of p-H3, in the brains and kidneys of 10 and 15-day-old R26CMER/+ and wild-type mice, 24 hrs post Myc activation by tamoxifen.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of microbiology, tissue culture, molecular biology, chemistry, biochemistry, recombinant DNA technology, and bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

In one aspect of the invention there is provided a nucleic acid molecule comprising a nucleic acid sequence encoding a myc transcription factor, cyclin T1 or cyclin-dependent kinase 9 (CDK9) for use as a medicament.

In another aspect of the invention, there is provided a nucleic acid construct comprising a nucleic acid sequence encoding at least one of a myc transcription factor, cyclin T1 and cyclin-dependent kinase 9 (CDK9) for use as a medicament. In a preferred embodiment, the nucleic acid sequence encodes a myc transcription factor and a cyclin T1

In certain embodiments, cyclin T1 may be replaced with cyclin K and/or cyclin T2. In further embodiments, the nucleic acid molecule may further comprise cyclin K and/or cyclin T2. It will be understood that the same may apply to other aspects of the invention. The amino acid and nucleic acid sequences of cyclin K and cyclin T2 are given as SEQ ID NO: 18 and 19, and SEQ ID NO: 20 and 21, respectively; these, or a functional variant thereof, may be used in the invention.

Cyclin T1 is encoded by the ccnt1 gene and such terms are used interchangeably throughout the application.

In one embodiment, the nucleic molecule is a ribonucleotide (RNA) molecule. Accordingly, in one embodiment of the invention there is provided a ribonucleotide, preferably an mRNA encoding a myc transcription factor, a cyclin T1 or cyclin-dependent kinase 9 (CDK9). wherein the mRNA is modified, preferably chemically modified (i.e. a modRNA). In a preferred embodiment the mRNA is modified to avoid detection by the Toll-receptor pathways, which would otherwise lead to the activation of the innate immunity pathway and cell death.

In another embodiment, there is provided an RNA molecule, preferably an mRNA molecule, encoding at least one of a myc transcription factor, a cyclin T1 and a cyclin-dependent kinase 9 (CDK9). Again, preferably the mRNA is modified, preferably chemically modified (i.e. a modRNA). More preferably the RNA molecule encodes a myc transcription factor and a cyclin T1 and/or a cyclin-dependent kinase 9 (CDK9). For example, the RNA molecule encodes a myc transcription factor and a cyclin T1; or a myc transcription factor and CDK9; or a myc transcription factor and a cyclin T1 and a CDK9. Such an RNA molecule is known as bicistronic or polycistronic RNA molecule. The bicistronic or polycistronic RNA molecules of the invention may further comprises at least one sequence that allows self-cleavage/self-processing of the myc and cyclin T1 and/or CDK9 upon or following transcription. Examples of suitable sequence are described below, and may include a ribosomal skipping sequence (such as P2A) or a t-RNA sequence.

In one embodiment, myc is selected from c-myc, l-myc or n-myc. In a most preferred embodiment, myc is c-myc. Preferably, the (unmodified) RNA sequence of c-myc comprises or consists of a sequence as defined in SEQ ID NO: 15 or a functional variant thereof. In an alternative embodiment, myc is an inducible form of myc as defined below. Where the myc is 1-myc or n-myc, the DNA sequence that encodes the unmodified mRNA sequence of the 1-myc or n-myc may comprise or consist of a sequence as defined in SEQ ID NO: 22 or SEQ ID NO: 24 respectively, or a functional variant thereof. The amino acid sequences of 1-myc and n-myc are given as SEQ ID NO: 23 and 25 respectively.

In one embodiment, the cyclin T1 RNA sequence comprises or consists of SEQ ID NO: 13.

In another embodiment, the CDK9 RNA sequence comprises or consists of SEQ ID NO: 14.

As explained above, preferably the RNA sequence of myc, cyclin T1 or CDK9 has one or more modifications. In one embodiment, the modification may be selected from a cap modification, a tail modification, a nucleoside modification and/or a UTR modification (Young and Chung, 2015; Expert Opinion Biological Therapy, 15(9) 1337-1348 incorporated herein by reference).

In a preferred embodiment, the cap modification comprises the introduction of an anti-reverse-cap analogue (ARCA) comprising a modified cap structure containing a 5′-5′ triphosphate bridge, wherein this modification increases the efficiency of mRNA translation. More preferably, the ARCA comprises a high number of modified cap structures and elongated 5′-5′ phosphate bridges, wherein this increases the efficiency of mRNA translation as well as the stability of the mRNA.

In a further preferred embodiment, the tail modification comprises a poly(A) tail comprising at least 20 poly(A)s, wherein this increases the efficiency of mRNA translation and stability of the mRNA.

In another preferred embodiment, the nucleoside modification is the introduction of a modified nucleoside selected from one or more of the following: 2′-O methyl nucleoside, pseudouridine, 2-thiouridine, 5-methylcytidine, 6-methyladenosine and inosine, wherein the modification reduces immune activation, preferably TLR-mediated immune activation.

In a further preferred embodiment, the UTR modification comprises replacing unstable non-coding sequences (for example, AU-rich sequences in the UTR) with non-coding sequences of mRNAs that are known to be stable (for example, 3′UTR of the human globin gene), wherein the modification improves stability of the mRNA.

In another embodiment, the modification is codon optimisation.

Methods for synthesising modified RNA molecules are known in the art—for example, as described in Kondrat et al. 2017.

In a particularly preferred embodiment, the modification comprises a cap modification and at least one modified nucleoside, preferably the modification of uracil to pseudouridine and/or the modification of cytidine to 5-methylcytidine. Accordingly, in one embodiment, the RNA sequence of myc may comprise a sequence as defined in SEQ ID NO: 37 or a functional variant thereof; the RNA sequence of cyclin-T1 may comprise a sequence as defined in SEQ ID NO: 16 and the RNA sequence of CDK9 may comprise a sequence as defined in SEQ ID NO: 17.

In an alternative embodiment, the nucleic acid molecule is a nucleic acid construct or vector (such terms may be used interchangeably). Accordingly, in one embodiment there is provided a vector comprising a nucleic acid sequence encoding a myc transcription factor operably linked to a regulatory sequence. The sequence of myc is described above.

In one embodiment, the vector comprises a nucleic acid sequence encoding an inducible form of myc. By “inducible” is meant that the expression and/or activity of the myc transcription factor is responsive to the presence or absence of an external stimuli. That is, expression and/or activity of the myc transcription factor can be turned on or off by the presence or absence of an external stimuli. Preferably the external stimuli acts as a reversible switch, meaning that expression and/or activity can be turned on and then off again by the presence of the external stimuli.

In one embodiment, the activity of myc may be regulated using a myc fusion protein. Accordingly, in one embodiment, the myc transcription factor is a fusion protein. For example, myc may be fused to a steroid or thyroid binding domain, such as for example, an estrogen receptor (ER), glucocorticoid receptor (GR), progesterone receptor (PR), thyroid receptor (TR), mineralocorticoid receptor (MR) or androgen receptor (AR) ligand binding domain. More preferably, the steroid or thyroid binding domain may be modified. For example, the ER binding domain may be modified to bind tamoxifen or the PR binding domain may be modified to bind RU486.

In a preferred example, myc is fused to a modified hormone-binding domain of the estrogen receptor—called ER^{T2} as described in Murphy et al. 2008, which is incorporated herein by reference. In this system binding of 4-hydroxytamoxifen (4-OHT) to the ER binding domain leads to a conformational change and activation of the myc fusion protein. In one embodiment, the inducible myc transcription factor is defined in SEQ ID NO: 1 or 10 or a functional variant thereof. In a preferred embodiment, the nucleic acid sequence encoding the inducible myc transcription factor comprises SEQ ID NO: 2 or 9 or a functional variant thereof.

The term “operably linked” as used throughout refers to a functional linkage between the promoter sequence and the gene of interest, such that the promoter sequence is able to initiate transcription of the gene of interest.

In one embodiment, the regulatory sequence is a promoter, preferably a constitutive or inducible promoter. An inducible promoter may be used with an inducible or non-inducible form of myc, but preferably an inducible promoter is used instead of an inducible form of myc.

One example of an inducible promoter is the Tet-On or Tet-Off promoter system. Accordingly, in one embodiment, the promoter is a Tet-inducible promoter. In a further embodiment, the vector may additionally comprise a tetracycline-controlled transactivator (tTA) or a reverse tetracycline-controlled transactivator (rtTA). Preferably, the tTA or rtTA are operably linked to a regulatory sequence such as a promoter. Suitable promoters would be known to the skilled person. Alternatively, the tTA or rtTA may be provided as a separate vector.

According to all aspects of the invention, the term “regulatory sequence” is used interchangeably herein with “promoter” and all terms are to be taken in a broad context to refer to regulatory nucleic acid sequences capable of effecting expression of the sequences to which they are ligated. The term “regulatory sequence” also encompasses a synthetic fusion molecule or derivative that confers, activates or enhances expression of a nucleic acid molecule in a cell, tissue or organ.

The term “promoter” typically refers to a nucleic acid control sequence located upstream from the transcriptional start of a gene and which is involved in the binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences.

A “constitutive promoter” refers to a promoter that is transcriptionally active during most, but not necessarily all, phases of growth and development and under most environmental conditions, in at least one cell, tissue or organ. Examples of constitutive promoters include the CAG promoter, the Rosa26 promoter, the ubiquitin promoter, the actin promoter, the tubulin promoter, the GAPDH promoter, as well as the CMV, PGK, SV40, and EF1A promoters.

For the identification of functionally equivalent promoters, the promoter strength and/or expression pattern of a candidate promoter may be analysed for example by operably linking the promoter to a reporter gene and assaying the expression level and pattern of the reporter gene in various tissue. Suitable well-known reporter genes are known to the skilled person and include for example beta-glucuronidase or beta-galactosidase.

In one embodiment, the regulatory sequence of the vector is a promoter, preferably the CAG promoter, as defined in SEQ ID NO: 3 or a functional variant thereof.

In a further embodiment, the vector further comprises a nucleic acid sequence encoding cyclin T1 and/or cyclin-dependent kinase 9 (CDK9). In one embodiment, the nucleic acid sequence encoding cyclin T1 and/or cyclin-dependent kinase 9 (CDK9) are operably linked to the same regulatory sequence. Preferably in this embodiment the vector may comprise at least one sequence that allows self-cleavage/self-processing of the myc and cyclin T1 and/or CDK9 upon transcription such that each of myc and the cyclin T1 and/or CDK9 can be expressed from a single promoter.

In one embodiment, to allow two proteins to be expressed as individual proteins from a single mRNA molecule, ribosomal skipping sequences may be added to the 5′ and/or 3′ end of the individual proteins (for example, myc and the cyclin T1 and/or CDK9 sequences). During translation, when the ribosome encounters a ribosomal skipping sequence it is prevented from creating the peptide bond with the last proline in the ribosomal skipping sequence. As a result, translation is stopped, the nascent polypeptide is released and translation is re-initiated to produce a second polypeptide. This results in the addition of a C-terminal ribosomal skipping sequence (or the majority of such a sequence) to the first polypeptide chain, and an N-terminal proline to the next polypeptide. Accordingly, in a further embodiment, the nucleic acid construct comprises at least one ribosomal skipping sequence. In one example, the ribosomal skipping sequence is a 2A-like peptide, such as P2A (comprising ATNFSLLKQAGDVEENPGP, SEQ ID NO: 26), E2A (comprising QCTNYALLKLAGDV ESNPGP, SEQ ID NO: 27) or F2A (comprising VKQTLNFDLLKLAGDVESNPGP, SEQ ID NO: 28). In an alternative embodiment, the nucleic acid construct may comprise at least one 2A-like peptide which provides a proteolytic cleavage site, for example T2A (comprising EGRGSLLTCGDVEENPGP, SEQ ID NO: 29).

Alternatively, tRNA sequences may be used to allow multiple RNAs to be produced from a single engineered polycistronic gene consisting of tandemly arrayed tRNA-RNA units. After the polycistronic gene is transcribed by endogenous transcriptional machinery, the endonucleases RNAse P and RNAse Z (or RNAse E in bacterium) recognise and specifically cleave the tRNAs at specific sites at the 3′ and 5′ ends, releasing mature RNAs and tRNAs. Advantageously, tRNAs and their processing system are virtually conserved in all living organisms and therefore this method can be used in all known species. In particular, this technology can allow for the production of multiple excised mature gRNAs which can direct Cas9 to multiple targets, wherein the polycistronic gene contains tandemly arrayed tRNA-gRNA units, where each gRNA contains a target-specific spacer and a conserved gRNA scaffold (see Xie et al, 2015). Accordingly, in a further embodiment, the nucleic acid construct comprises at least one tRNA sequence.

Accordingly, in one embodiment, at least one of myc and cyclin T1 and/or CDK9 may be flanked at the 5′ or 3′ ends by a ribosomal skipping sequence and/or a proteolytic cleavage site. The construct may further comprise linker sequences between one of myc, cyclin T1 and CDK9. In one example, the linker may be a GS linker.

In an alternative embodiment, the nucleic acid sequence encoding cyclin T1 and/or cyclin-dependent kinase 9 (CDK9) is operably linked to a second regulatory sequence. A regulatory sequence is described above. In one example, the regulatory sequence is the troponin T promoter, preferably as defined in SEQ ID NO: 4 or a functional variant thereof.

In a further embodiment of the invention, the nucleic acid construct may further comprise at least one UTR, preferably a 5′ and 3′ UTR. In a further embodiment, at least one of myc and cyclin T1 and/or CDK9 may be flanked at their 5′ and 3′ ends with a UTR. In one embodiment, the sequence of the 5′ UTR preferably comprises the consensus sequence: GCCRCC, where R is a purine, preferably A. In one example, the sequence of the 5′UTR comprises or consist of SEQ ID NO: 38 or a variant thereof. A variant is defined above.

In a preferred embodiment, the cyclin T1 nucleic acid encodes a protein as defined in SEQ ID NO: 5. More preferably, the cyclin T1 nucleic acid comprises or consists of a sequence as defined in SEQ ID NO: 6 or a functional variant thereof.

In a further embodiment, the vector comprises at least one terminator sequence, which marks the end of the operon causing transcription to stop. A suitable terminator sequence would be well known to the skilled person.

Similarly in a preferred embodiment, the CDK9 nucleic acid encodes a protein as defined in SEQ ID NO: 7. More preferably, the cyclin T nucleic acid comprises or consists of a sequence as defined in SEQ ID NO: 8 or a functional variant thereof.

In one embodiment, the vector is a viral vector. More preferably, the viral vector is selected from adenoviruses, adeno-associated viruses (AAV), alphaviruses, flaviviruses, herpes simplex viruses (HSV), measles viruses, rhabdoviruses, retroviruses, lentiviruses, Newcastle disease virus (NDV), poxviruses, picornaviruses and hybrids thereof. In one embodiment, the vector is an adeno-associated virus (AAV). In a further preferred embodiment, the AAV may be selected from serotype 1, 2, 3, 4, 5, 6, 7, 8, 9, 10 or 11. The skilled person would understand that serotypes differ in their tropism and as such the selection of the target tissue will depend on the target tissue to be infected. In one example, where the target tissue is the heart, the AAV may be selected from serotypes 1, 8 and 9, and most preferably is AAV9. In another example, where the target tissue is the brain or CNS, the AAV may be selected from serotypes 1, 2, 4, 5, 8 and 9. In another example where the target tissue is the kidney, the AAV may be AAV2.

In an alternative embodiment, the vector is a non-viral vector, such as a plasmid. In this embodiment, a non-viral vector may be delivered to a target cell or tissue using transfection. Examples of suitable transfection techniques would be known to the skilled person and include chemical and physical transfection. In one embodiment, chemical transfection includes the use of calcium phosphate, lipid or protein complexes. In one example, the non-viral vector may be combined with a lipid solution to result in the formation of a liposome or lipoplex. In another embodiment, physical transfection means include electroporation, microinjection or the use of ballistic particles. In a further example, bacteria can be used to deliver a non-viral vector to a target cell or tissue. This is known as bactofection.

In a further aspect of the invention, there is provided a RNA molecule obtained or obtainable by transcription of the nucleic acid construct described above. In one embodiment, where the nucleic acid construct comprises sequences encoding myc and cyclin T1 and/or CDK9, the RNA molecule obtained or obtainable by transcription of the nucleic acid construct is a bicistronic RNA.

In another aspect of the invention there is provided a host cell comprising the vector described above.

In another aspect of the invention, there is provided a method of increasing at least one of proliferation, mitosis, cytokinesis in a cell, the method comprising introducing and expressing a vector or introducing at least one mRNA molecule as described herein to a cell. Preferably, where a mRNA molecule is introduced the method comprises introducing a first mRNA molecule encoding a myc transcription factor as described above and a second mRNA molecule encoding a cyclin T1 and/or cyclin-dependent kinase 9 (CDK9). The first mRNA molecule may be introduced before, after or concurrently with the second mRNA molecule.

As used herein an “increase” in proliferation may refer to an increase in proliferation of at least 5%, 10%, 20%, 25%, 30%, 35%, 40%, 45%, 50%, 55%, 60%, 65%, 70%, 75%, 80%, 85%, 90%, 95%, 100% or more compared to the level in a control. As used herein a control may be considered a cell that does not comprise or express a vector of the invention. In one embodiment, an increase in cell proliferation may be assessed by an increase in cell number.

In another aspect of the invention there is provided a method of increasing organ size, the method comprising introducing and expressing a vector or introducing at least one mRNA molecule as described herein to at least one cell in the organ. Preferably, where a mRNA molecule is introduced the method comprises introducing a first mRNA molecule encoding an inducible myc transcription factor as described above and a second mRNA molecule encoding a cyclin T1 and/or cyclin-dependent kinase 9 (CDK9). The first mRNA molecule may be introduced before, after or concurrently with the second mRNA molecule.

In a preferred embodiment, the organ is the heart, a component of the CNS or the brain.

In a further aspect of the invention, there is provided the use of a vector or at least one mRNA molecule as described herein to increase at least one of cell proliferation, mitosis, cytokinesis and organ size.

As used herein, an “increase” in organ size may refer to an increase in organ size of at least 5%, 10%, 20%, 25%, 30%, 35%, 40%, 45%, 50%, 55%, 60%, 65%, 70%, 75%, 80%, 85%, 90%, 95%, 100% or more compared to the level in a control. As used herein a control may be considered a cell or organ that does not comprise or express a vector or mRNA molecule of the invention.

The “host cell” or “cell” as used herein may be eukaryotic, and may include bacterial cells, fungal cells such as yeast, plant cells, insect cells, or mammalian cells. In one example, the host cell may be a cell with a reduced or limited capacity to regenerate, wherein preferably the host cell is from an adult organism. Examples of such cells include cells from the kidney, heart and brain. Accordingly, in one example, the cell is a cardiomyocyte. In an alternative example the cell is a neuron.

In another aspect of the invention there is provided a transgenic organism where the transgenic organism expresses a vector or mRNA molecule of the invention. Again, the organism is any prokaryote or eukaryote.

In one embodiment, the progeny organism is transiently transformed with the vector or mRNA molecule. In another embodiment, the progeny organism may be stably transformed with the vector described herein and comprises the exogenous polynucleotide which is heritably maintained in at least one cell of the organism. The method may include steps to verify that the construct or vector is stably integrated.

In a further aspect of the invention, there is provided an organism obtained or obtainable by the methods described herein.

The term “organism” as used herein refers to any prokaryotic or eukaryotic organism. Some examples of eukaryotes include a human, a non-human primate/mammal, a livestock animal (e.g. cattle, horse, pig, sheep, goat, chicken, camel, donkey, cat, and dog), a mammalian model organism (mouse, rat, hamster, guinea pig, rabbit or other rodents), an amphibian (e.g., Xenopus), fish, insect (e.g. Drosophila), a nematode (e.g., C. elegans), a plant, an algae, a fungus. Examples of prokaryotes include bacteria (e.g. cyanobacteria) and archaea. In a most preferred embodiment, the organism is a human.

In another aspect of the invention there is provided a composition comprising a combination of a first vector, wherein the first vector comprises a nucleic acid sequence encoding an inducible myc transcription factor operably linked to a regulatory sequence, as described herein, and a second vector comprising a nucleic acid sequence encoding a cyclin T1 and/or cyclin-dependent kinase 9 (CDK9) operably linked to a regulatory sequence, as also described above.

In a further aspect of the invention there is provided a composition comprising a first mRNA molecule encoding a myc transcription factor as described above and a second mRNA molecule encoding a cyclin T1 and/or cyclin-dependent kinase 9 (CDK9). In an alternative aspect of the invention there is provided a comprising a bi or polycistronic RNA molecule as described above.

Optionally the composition may further comprise a pharmaceutically acceptable carrier. Such pharmaceutically acceptable carriers may comprise excipients and other components which facilitate processing of the active compounds into preparations suitable for pharmaceutical administration.

In a further aspect of the invention there is provided a vector, mRNA molecule or composition as described herein for use as a medicament. In a further aspect there is provided a vector, mRNA molecule or composition as described herein for use in the treatment of a condition characterised by the loss of cells or cell death, preferably in the heart, CNS, brain or kidney.

Where the tissue concerned is the heart, regeneration of cardiomyocyte tissues is most relevant for heart attacks and reduced ejection fraction as the addition of cardiomyocytes has the potential to improve heart functionality. In reduced ejection fraction the heart tissue becomes weaker or thinner thus reducing its ability to pump blood around the body. In a heart attack (myocardial infarction) cardiomyocytes die as a result of oxygen loss to heart tissues (e.g. due to an artery blockage) and thus reduced heart function (or death) can result. Accordingly, in one embodiment, the condition characterised by loss of cells or cell death in the heart is a heart disease, such as a heart attack (or myocardial infarction) or reduced ejection fraction.

In another embodiment, the condition is characterised by the loss of cells or cell death in the CNS or brain (i.e. neuronal loss). Examples of such conditions include spinal cord injury, stroke and neurodegenerative disorders such as multiple sclerosis, Alzheimer's disease, Parkinson's disease, Huntington's disease and motor neurone disease (amyotrophic lateral sclerosis). Further examples of such conditions may include macular degeneration, glaucoma, retinopathy, muscular dystrophy, brain injuries caused by trauma, seizure disorders such as epilepsy, cancer such as brain tumours, and infections such as meningitis.

In a further aspect of the invention there is provided a method of therapy comprising administering a vector, mRNA molecule or composition as described herein to an individual or patient in need thereof. In a yet further aspect of the invention there is provided a method of treating a condition characterised by the loss of cells or cell death, preferably in the heart, CNS, brain or kidney, as described above, the method comprising administering a, preferably therapeutically effective, amount of a vector, mRNA molecule or composition as described herein to a patient or individual in need thereof.

A “therapeutically effective amount” as used herein may refer to an amount that is suitable to be therapeutically effective at the dosage and for the periods of time necessary to achieve the therapeutic purpose. The skilled person will appreciate that the amount to be administered will vary depending on such factors as the age, sex, weight of the individual. A therapeutically effective amount may also preferably be an amount that limits any unwanted side-effects on the treatment.

In one embodiment, introduction and expression of the vector described herein leads to ectopic expression of myc and/or P-TEFb. As used herein “ectopic” expression of myc may refer to an increase in the basal levels of myc and/or P-TEFb expression by at least 200% compared to a control.

In another preferred embodiment of the methods described herein, when the inducible myc transcription factor is a myc-fusion protein as described above, and more preferably where the myc transcription factor is fused to a steroid or thyroid binding domain, the method may further comprise administration of the steroid or thyroid.

In a preferred embodiment where the myc transcription factor is fused to an ER binding domain or a modified ER binding domain as described above, the steroid may be 4-OHT. Alternatively, the steroid is tamoxifen.

In a preferred embodiment, the steroid or thyroid is administered anything from one day to one or more weeks after administration of a vector of the invention. This time scale depends on the route of delivery of the steroid or thyroid, and suitable timescales would be known to the skilled person. In one embodiment, the steroid or thyroid is administered one, two, three, four, five, six or seven days after administration of the vector of the invention. Preferably in this embodiment the steroid or thyroid is administered in a vector. In an alternative embodiment, the steroid or thyroid is administered one, two, three, four, five, six or seven, eight, nine or ten weeks after administration of the vector of the invention. Preferably in this embodiment, the steroid or thyroid is administered orally.

Where the composition is used the first vector or first mRNA molecule may be introduced before, after or concurrently with the second vector or second mRNA molecule.

The term “variant” or “functional variant” as used throughout with reference to any of SEQ ID NOs refers to a variant nucleotide, ribonucleotide or protein sequence that retains the biological function of the full non-variant sequence. A functional variant also comprises a variant that has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence or ribonucleic acid sequence that result in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

As used in any aspect of the invention described throughout a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid, ribonucleic acid or amino acid sequence.

By “introducing” (of the vector or mRNA molecule of the invention) is meant administration of the vector or mRNA molecule.

Administration of the vector or the mRNA molecule may be accomplished by physical disturbance or generation of mRNA endocytosis by cationic carriers. Methods of physical disturbance include electroporation, gene guns, ultrasound or high-pressure injection. Methods of generation of mRNA endocytosis by cationic carriers such as self-assembled lipo-plexes or poly-plexes made up of cationic lipids or polymers including lipolexes, polyplexes, polycations and dendrimers. Suitable polycations include DEAE (diethylaminoethyl)-dextran, DOTMA/DOPE (1,2-dioeyl-3-trimethylammoniumchloride/1,2-dioleoyl-3-phodphoethanolamine), poly L-lysine, and PEI (polyethylene imine), and most preferably, DOTAP (1,2-dioleoyl-3-trimethylammonium propane).

Administration of the vectors or the mRNA molecule may be accomplished orally or parenterally. Similarly, administration of the external stimuli, such as steroid or thyroid of the invention may also be accomplished orally or parenterally. Methods of parenteral delivery include topical, intra-arterial, intramuscular, intracardiac, subcutaneous, intramedullary, intrathecal, intraventricular, intravenous, intraperitoneal, mucosal or intranasal administration. Most preferably however the vector or mRNA molecule(s) is administered by local or systemic injection. Local injection encompasses electroporation, gene gun, ultrasound and high pressure; whilst systemic injection encompasses vein injection, portal injection and artery injection. Where the target tissue is the heart, the vector or mRNA molecule(s) may be administered directly into the heart concurrently with a surgical procedure for a stent or coronary artery bypass, for example. Where the target tissue is the retina, direct administration to the retina is preferred (for example, retinal injection).

Of note, in a particularly preferred embodiment, when the mRNA molecule is administered by systemic injection it is administered with cationic lipid or polymer complexes (preferably PEGylated lipid or polymer complexes) to protect the mRNA molecule against nuclease activity and thus improve stability of mRNA.

In one embodiment, the mRNA molecule may be administered in the form of a nanoparticle, more preferably a liposome-protamine-RNA or LPR. An LPR comprises modified anionic mRNA which is mixed with polycation protamine to generate an mRNA/protamine complex which is then mixed with a liposome (comprising cationic lipid DOTAP and cholesterol) to form an LPR complex which is preferably PEGylated (Wang et al, 2013; Molecular Therapy, 21(2) 358-367). The resulting LPR is small in size (<100 nm) allowing for easy internalisation. Accordingly, in a further aspect of the invention there is provided a nanoparticle, preferably a LPR comprising a mRNA encoding at least one of an inducible myc transcription factor, a cyclin T1 and/or cyclin-dependent kinase 9 (CDK9), as described above. In a preferred embodiment the nanoparticle is administered by local or systemic injection.

Pharmaceutical compositions for oral administration can be formulated using pharmaceutically acceptable carriers known in the art in dosages suitable for oral administration. Such carriers enable the compositions to be formulated as tablets, pills, dragees, capsules, liquids, gels, syrups, slurries, suspensions, and the like suitable for ingestion by the subject.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous suspension injections can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilisers or agents which increase the solubility of the compounds to allow for the preparation of highly concentrated solutions. Pharmaceutical compositions may also include adjuvants to enhance or modulate antigenicity.

For topical or nasal administration, penetrants appropriate to the particular barrier to be permeated may be used in the formulation.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

As used herein, a control may be an individual, patient or cell that has not been treated with at least one vector of the invention.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting example.

### Example I

Myc is a basic helix-loop-helix-leucine zipper (bHLH-LZ) transcription factor that binds preferentially to specific sequences in the genome, termed E-boxes (Blackwood and Eisenman, 1991), via association with its bHLH-LZ heterodimerisation partner Max (Amati et al., 1993, 1992; Blackwood et al., 1992). Myc functions principally as a transcriptional activator by potentiating transcription initiation via association with various cofactors such as TRRAP (Bouchard et al., 2001; McMahon et al., 1998) and facilitating productive transcriptional elongation by promoting RNA PolII loading and via its association with positive transcription elongation factor (P-TEFb) (De Pretis et al., 2017; Eberhardy and Farnham, 2002; Kanazawa et al., 2003; Rahl et al., 2010). P-TEFb is comprised of Cdk9 and Cyclin T1 which are stringently regulated by various transcriptional and post transcriptional mechanisms (Jonkers and Lis, 2015; Peterlin and Price, 2006; Zhou et al., 2012; Zhou and Yik, 2006), and dynamically controlled by an association with inactivation complex comprised of 7SK snRNA, Larp7, MEPCE and HEXIM (Barboric et al., 2005; He et al., 2008; Yik et al., 2003). P-TEFb phosphorylates Serine 2 of the C-terminal Domain (CTD) of paused RNA PolII leading to productive elongation (Jonkers and Lis, 2015; Peterlin and Price, 2006; Zhou et al., 2012; Zhou and Yik, 2006).

Max is also the heterodimerisation partner of the Mxd family of transcription factors that act principally as transcriptional repressors through their interactions with HDASC2, SMC3 and Sin3A (Diolaiti et al., 2015). Since Mxd/Max heterodimers bind sites overlapping with Myc/Max and act as transcriptional repressors, it has been suggested that competition for Max by Myc versus Mxd proteins dictates transcriptional outputs at bound targets (Ayer et al., 1993; Kretzner et al., 1992).

Myc is a highly pleiotropic transcription factor which coordinates multiple transcriptional programmes involved in cell replication and differentiation, metabolism and apoptosis (Amati et al., 1993; Dang, 2013; Eilers et al., 1991; Evan et al., 1992; Roussel et al., 1991). With the development of animal models allowing for switchable ectopic Myc expression in vivo, it has become evident that Myc also governs diverse cell extrinsic processes required for tissue regeneration—such as angiogenesis, modulation of the local inflammatory and immune responses, invasion and migration—but in a manner that is tightly tailored to the tissue in which Myc is activated (Kortlever et al., 2017; Shchors et al., 2006; Sodir et al., 2011). Since deregulated and elevated Myc expression is a pervasive and causal attribute of most, perhaps all, tumours, understanding how tissue-specific responses to Myc are determined at a molecular level is imperative.

To determine how tissue-specificity controls the output of Myc activity, we generated a versatile, reversibly switchable mouse model in which supraphysiological levels of Myc are expressed comparably across different tissues. Using this model, we assessed Myc target gene binding and expression in distinct tissues at just 4 hours post activation of Myc. This short time scale limits any confounding normalization issues that might arise from global RNA amplification as well as avoids indirect gene expression changes coming from secondary transcriptional activation. This unique in vivo model allows us to identify essential differences between tissues in which Myc drives proliferation and tissues that exhibit no such proliferative response.

Results

The Capacity of Myc to Drive Proliferation is Tissue-Restricted

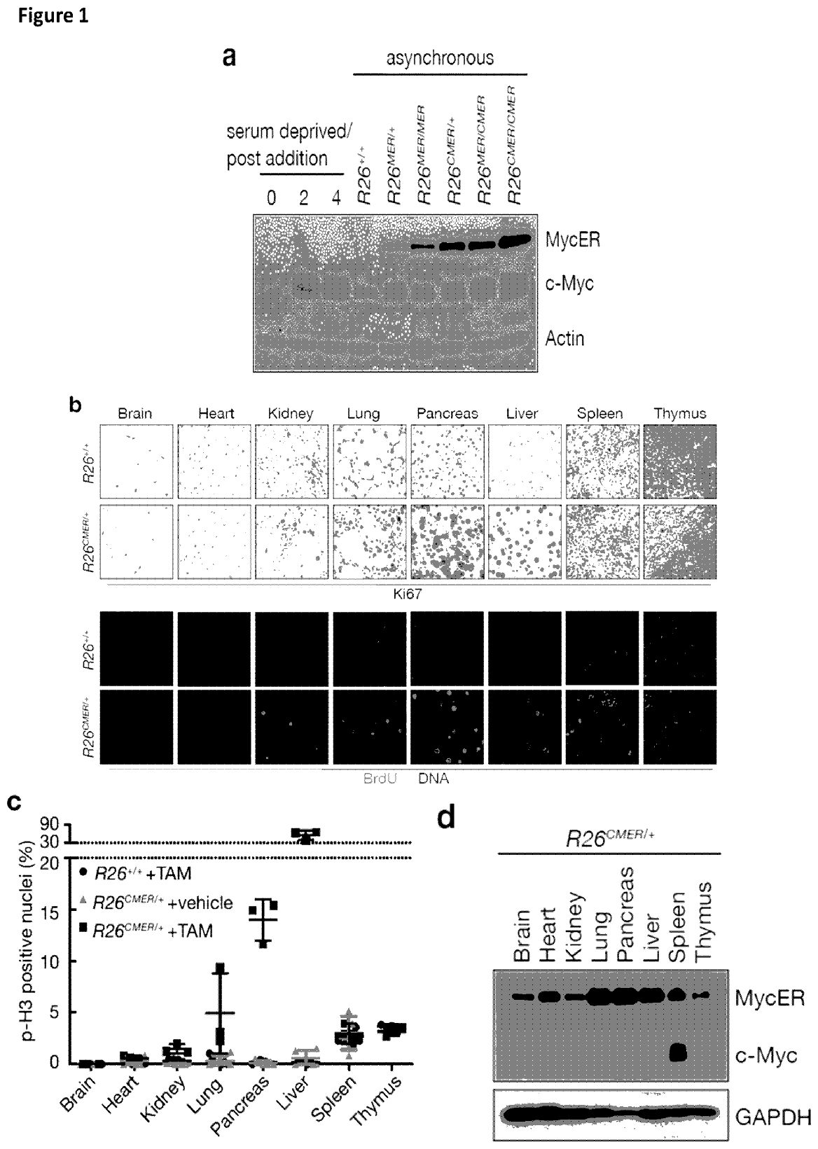

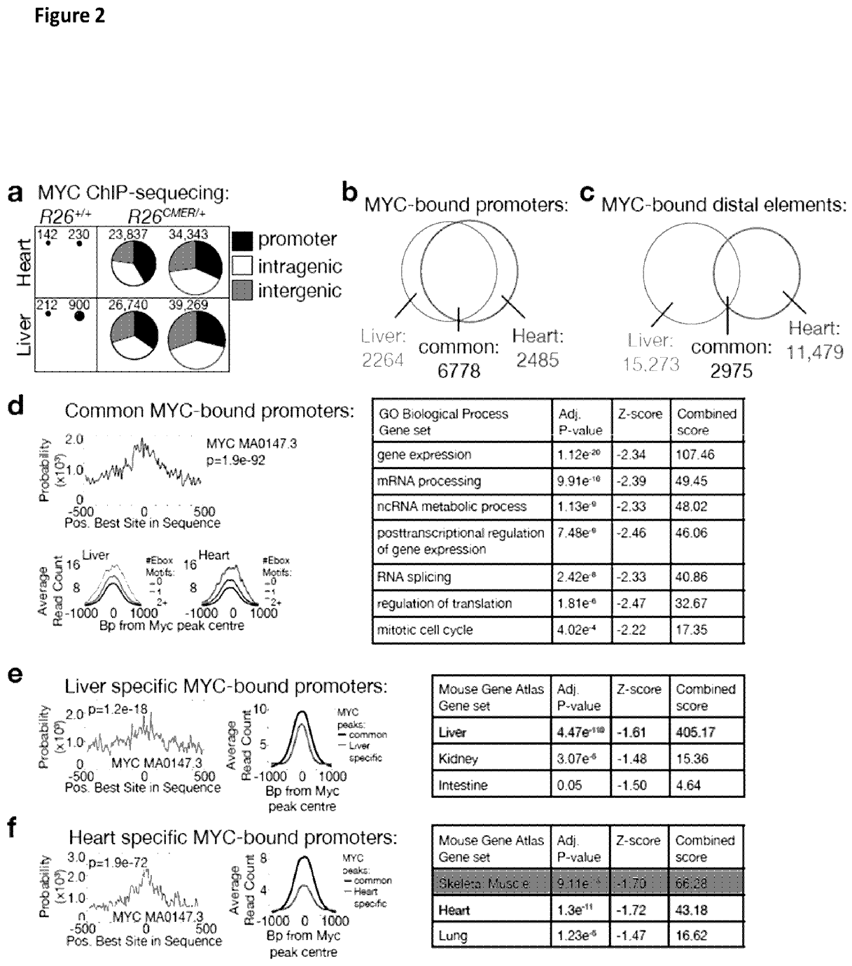

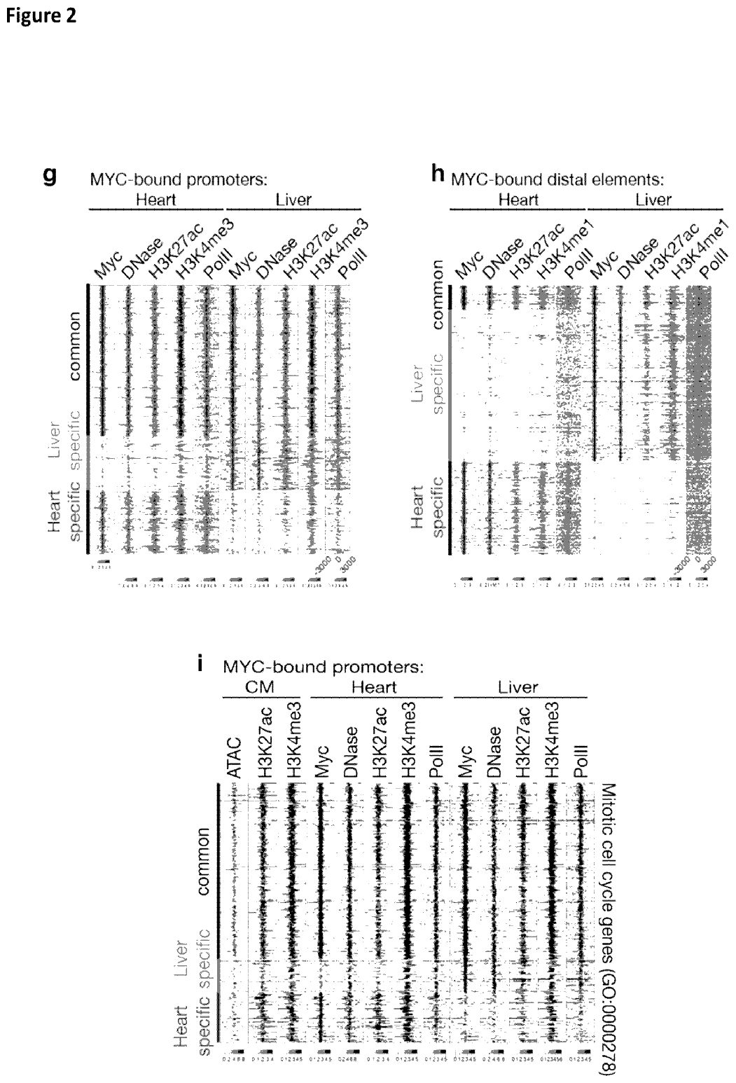

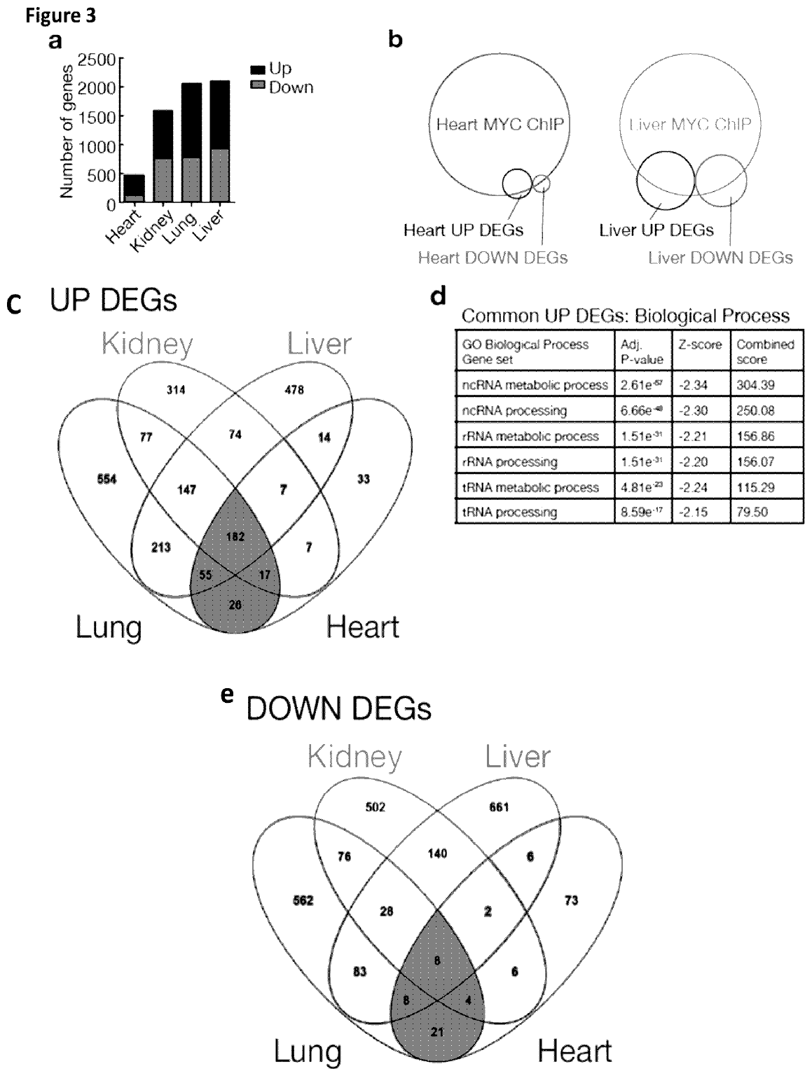

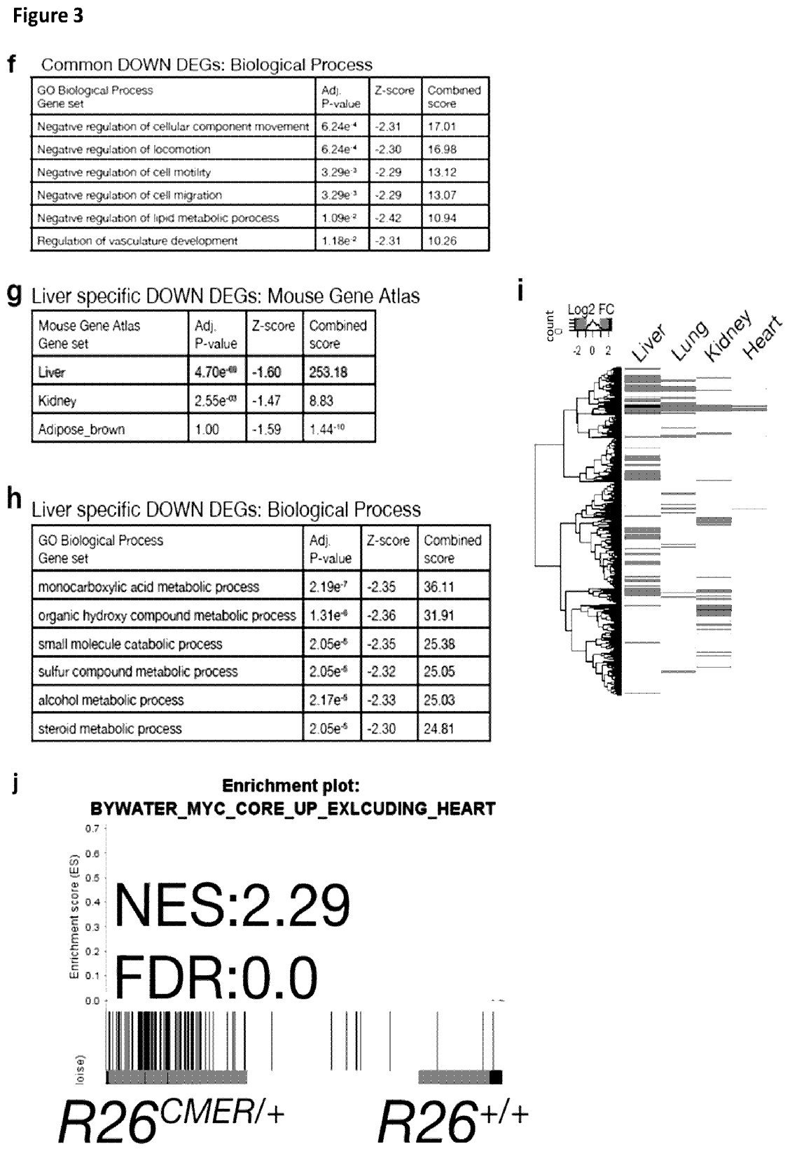

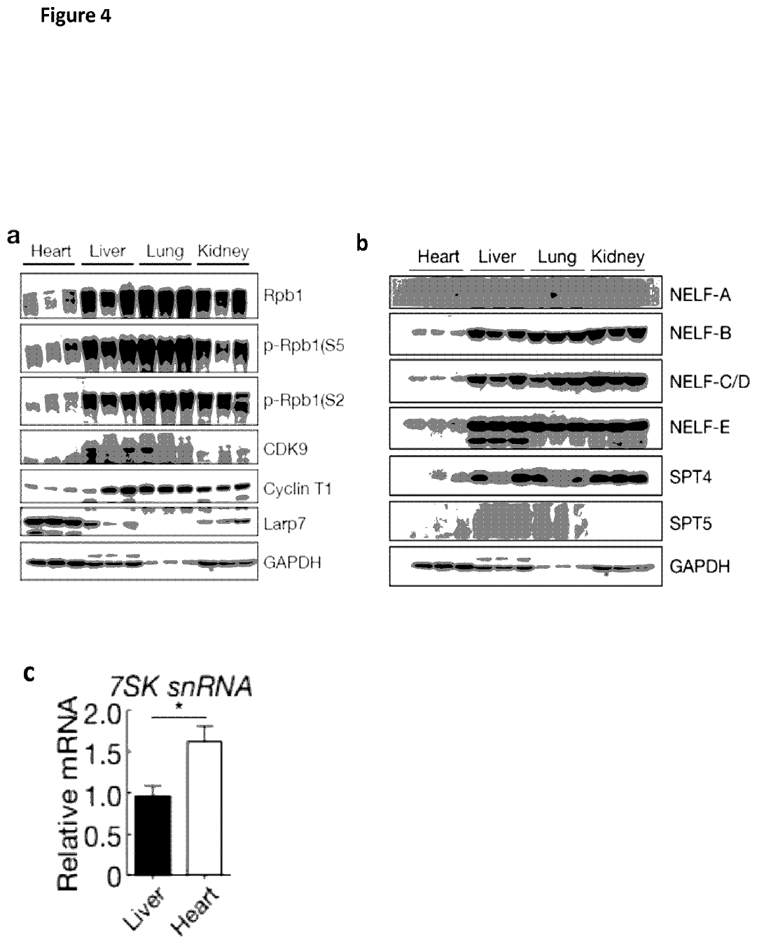

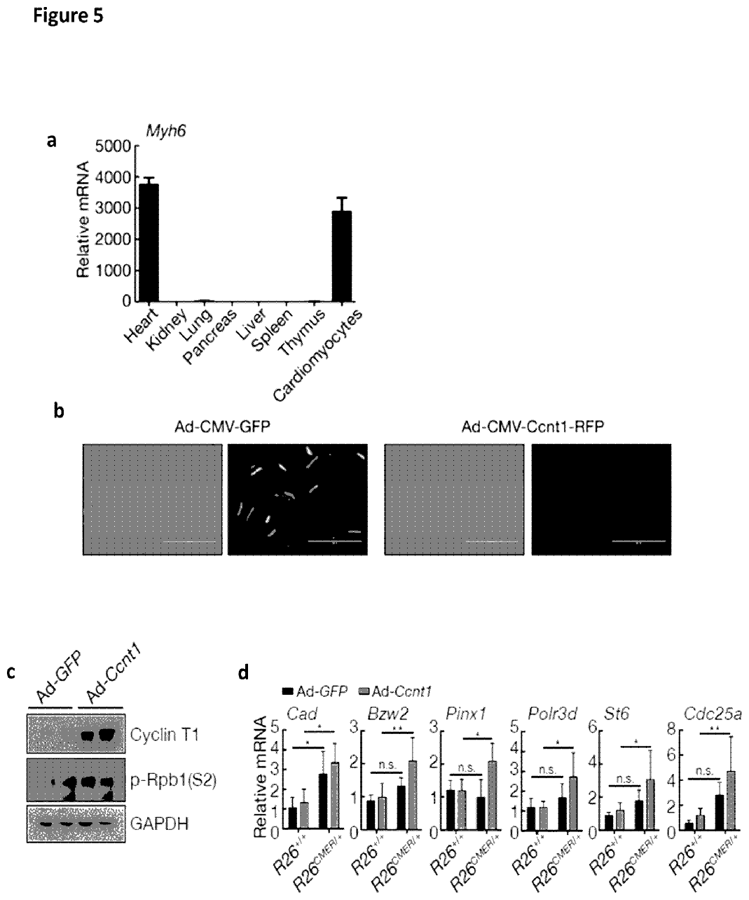

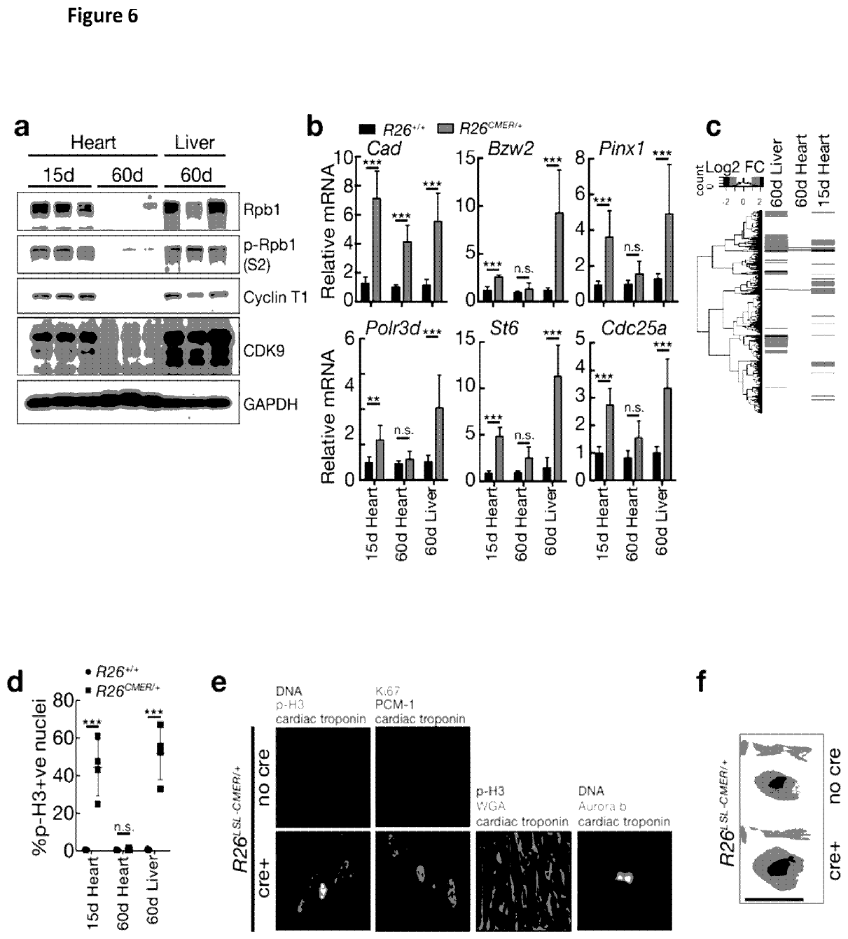

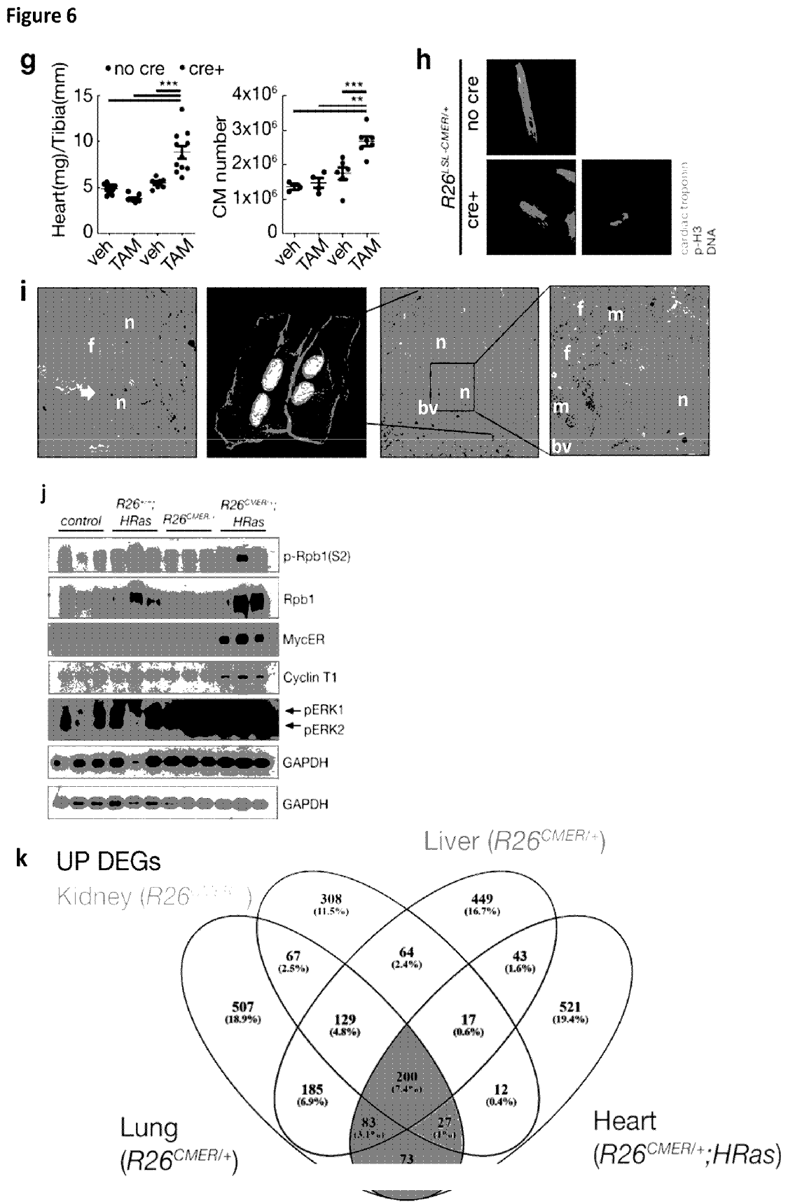

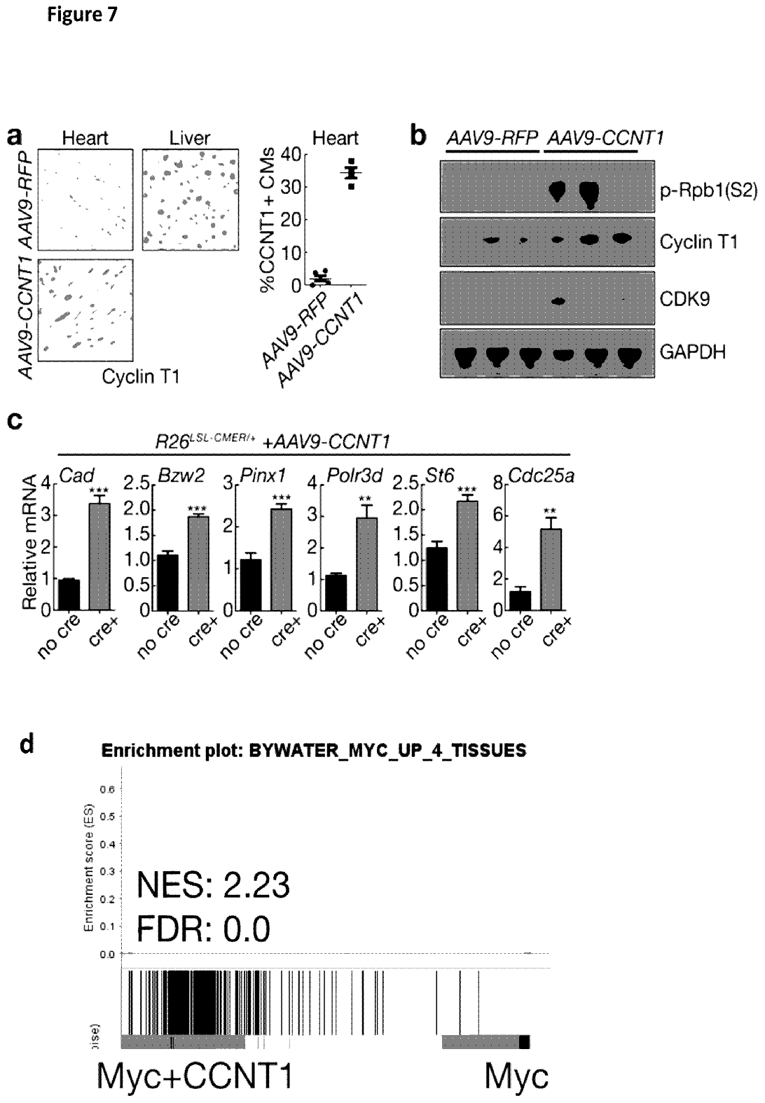

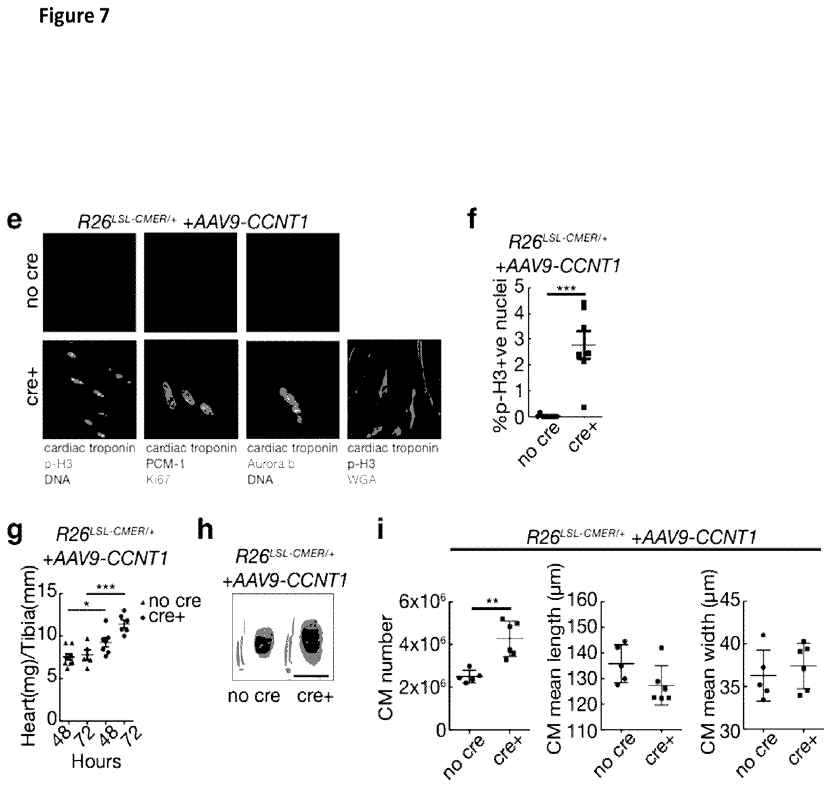

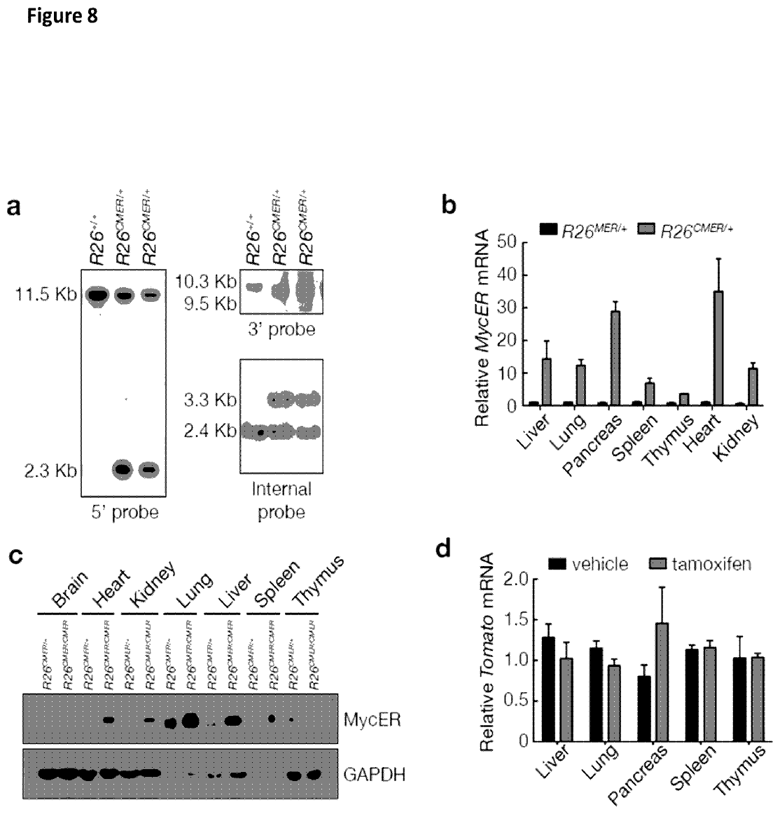

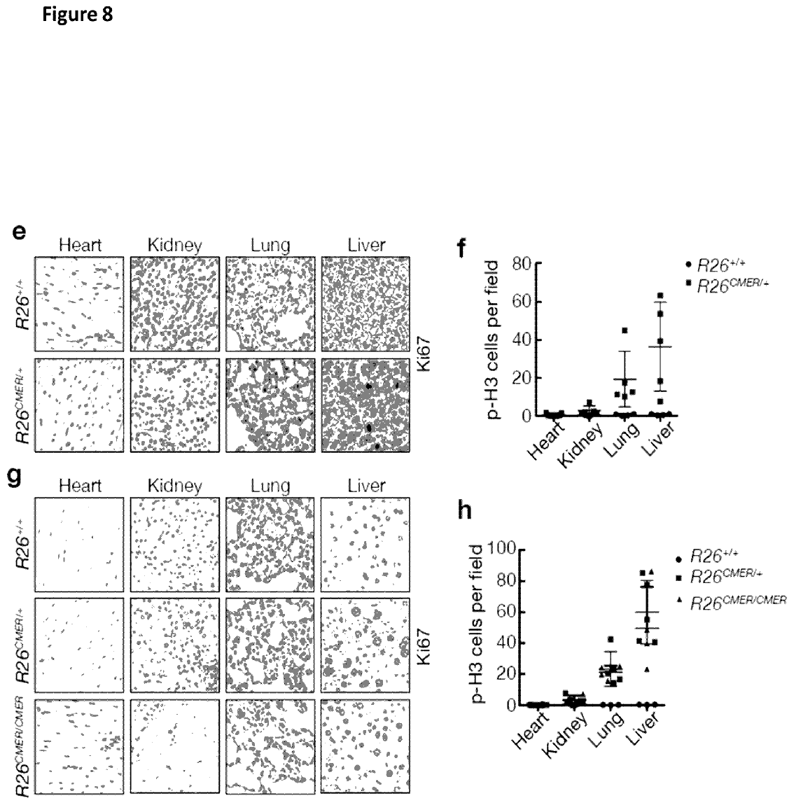

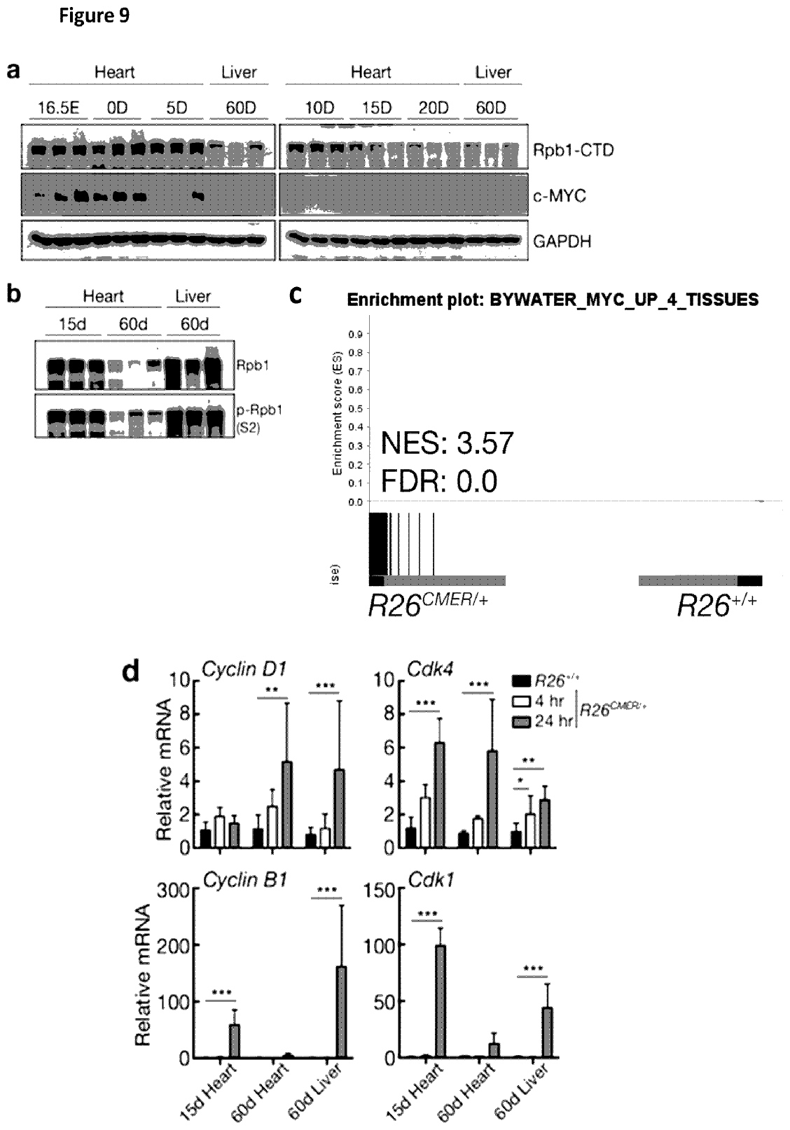

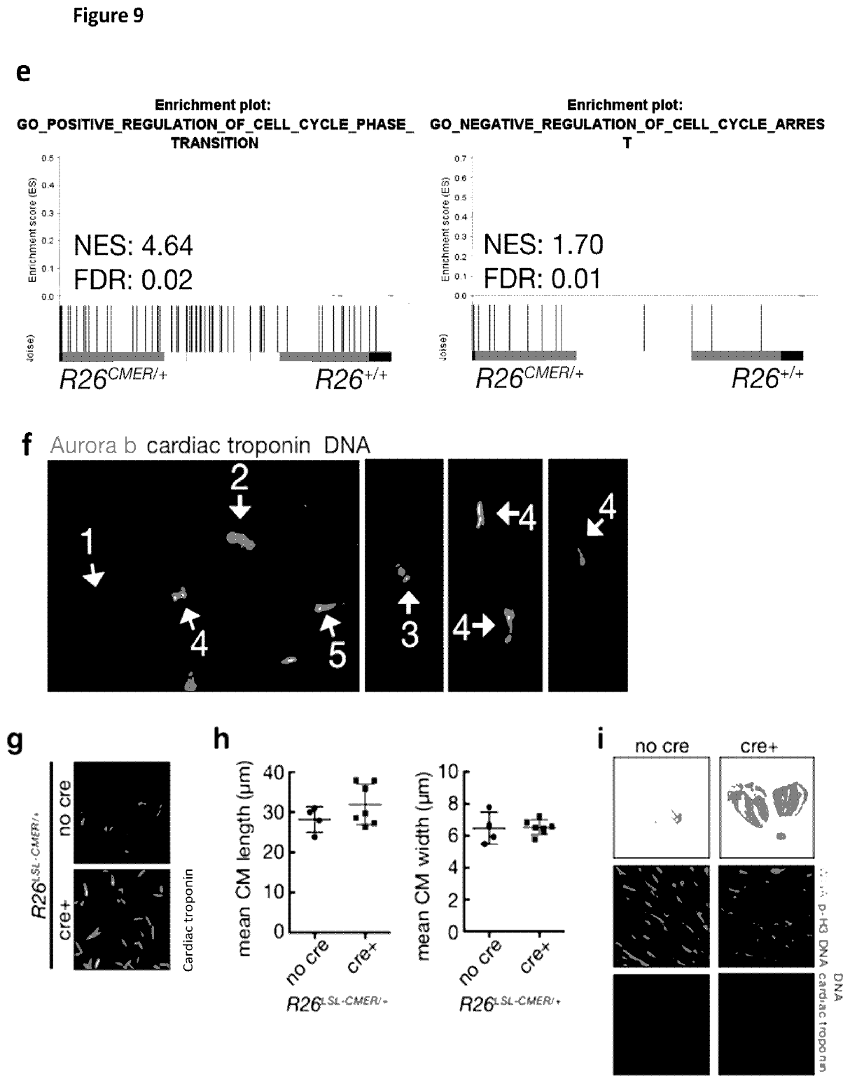

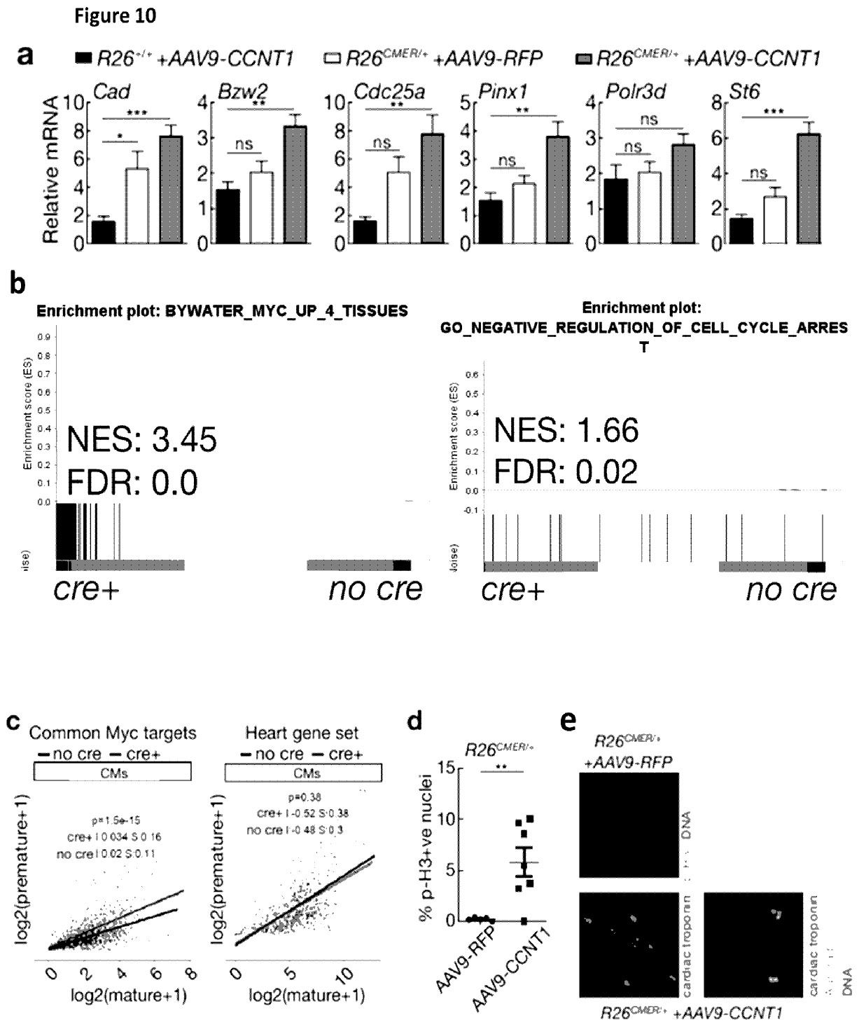

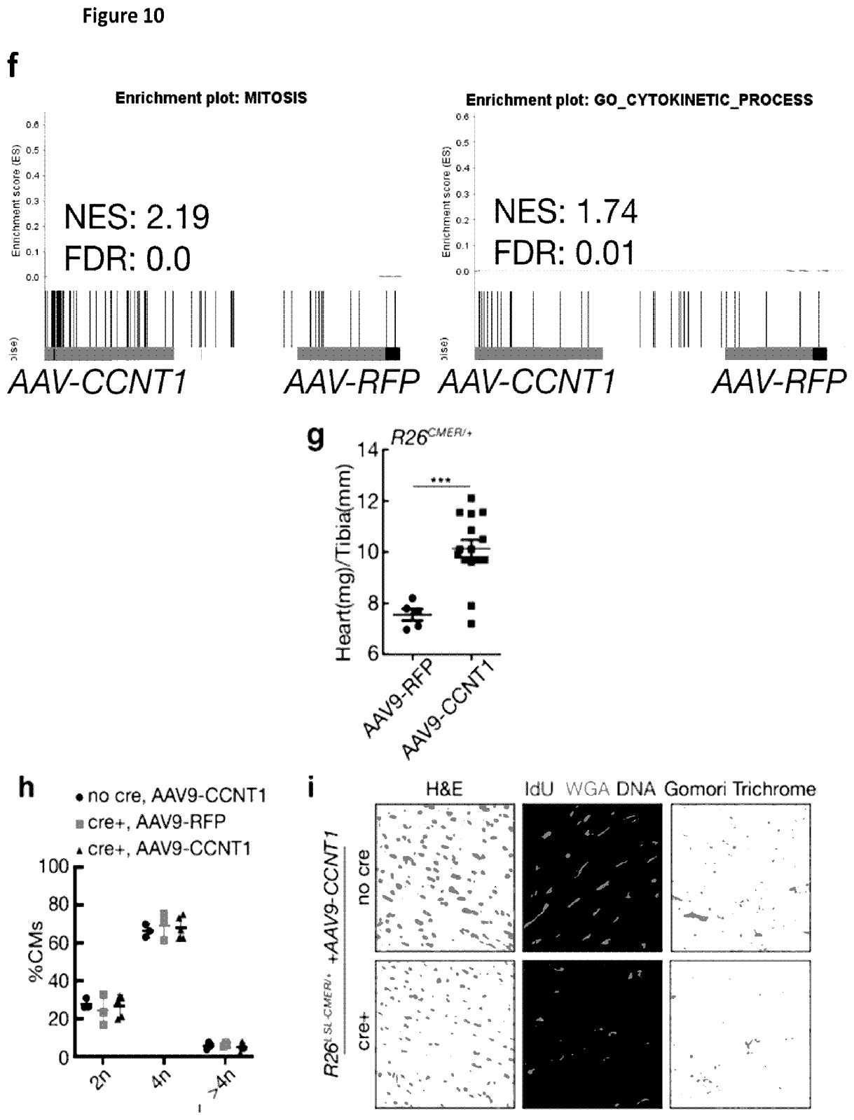

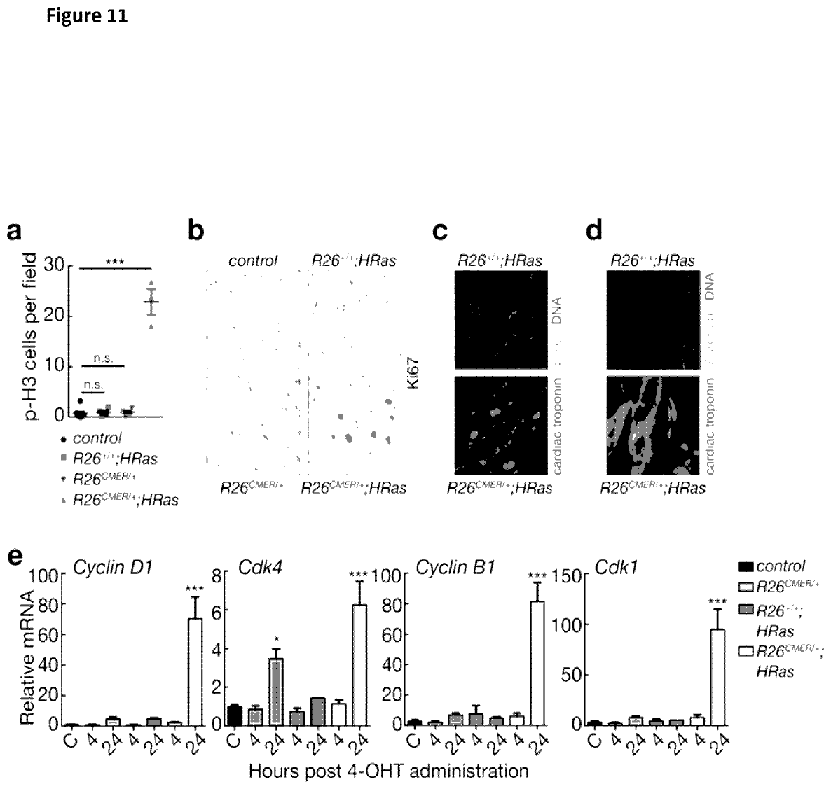

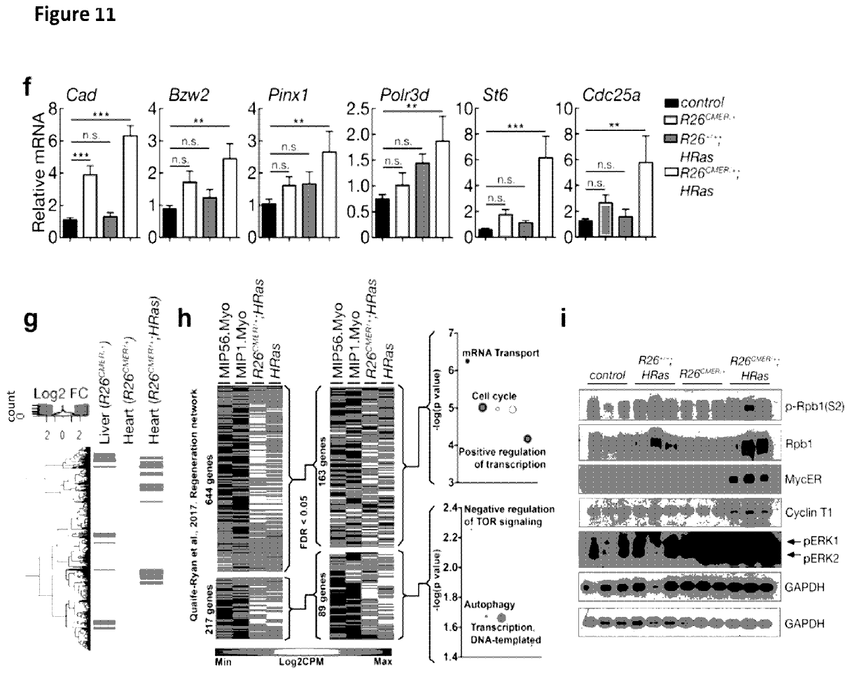

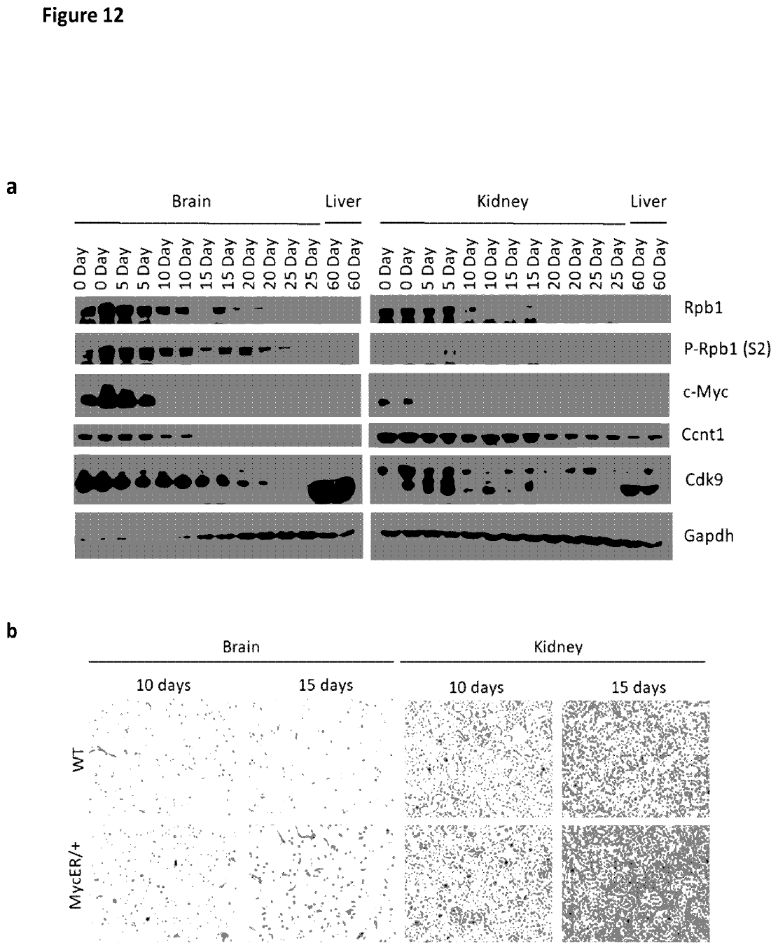

To compare the responses of different tissues to the acute activation of a similar level of Myc, we generated a mouse strain in which supraphysiological levels of the switchable Myc protein c-MycER^{T2} are ubiquitously expressed from a common promoter at comparable levels across tissues. In this knock-in mouse strain, the R26^{LSL-c-MycER} locus (R26^{MER}) (Murphy et al., 2008) is modified to include a CAG (chicken beta actin/CMV) enhancer to augment MycER^{T2} expression (FIG. 8a). Such R26_{CAG-LSL-c-MycERT2} mice were crossed into the Tg(Zp3-cre)93Knw strain in which Cre is active in the growing oocyte, effectively excising the LSL stop cassette from all adult tissues in the resultant R26^{CMER} mice (De Vries et al., 2000). Compared with the physiological level of Myc expressed in R26^{MER/+} cells (heterozygous for the R26^{MER} allele)(Murphy et al., 2008), R26^{CMER/+} cells express around 8-fold higher levels of MycER^{T2} RNA (FIG. 8b) in each tissue tested. R26^{CAG-LSL-MER} and R26^{LSL-MER} mice were interbred to generate an allelic series of ascending levels of MycER^{T2} expression (R26^{+/+}, R26^{LSL-MER/+}, R26^{LSL-MER/LSL-MER}, R26^{CAG-LSL-MER/+}, R26^{CAG-LSL-MER/LSL-MER} and R26^{CAG-LSL-MER/CAG-LSL-MER}). To measure directly relative levels of MycER^{T2} in this allelic series, mouse embryo fibroblasts (MEFs) were isolated from each strain and the LSL stop cassette excised by infection in vitro with a Cre expressing Adenovirus to engage constitutive MycER^{T2} expression. Western blot analysis of cell lysates confirmed an ascending allelic series of MycER^{T2} expression with homozygous R26^{MER} and R26^{CMER} cells expressing twice the level of their respective heterozygous counterparts (FIG. 1a). This allelic expression series was precisely mirrored in tissues from heterozygous and homozygous R26^{CMER} mice (FIG. 8c).

To rule out the possibility that high levels of Myc might modulate the Rosa26 promoter—and hence elicit artifactual feedback effects—we crossed R26^{CMER} mice to mice carrying a R26^{mTmG} (Gt(ROSA)26Sor^{tm4(ACTB-tdTomato,-EGFP)Luo}) reporter allele (Muzumdar et al., 2007). Systemic activation of MycER^{T2} in R26^{CAG/mTmG} mice had no impact on expression of either Tomato or MycER^{T2} transcripts in any tested tissues (FIG. 8d). Hence, elevated MycER^{T2} activity does not modulate activity of the Rosa26 promoter.

We next determined whether acute activation of MycER^{T2} elicits a proliferative response in tissues of R26^{CMER/+} mice. MycER^{T2} was activated for twenty-four hours by systemic administration of tamoxifen (Wilson et al., 2014) and proliferation assessed by immunohistochemical staining of Ki67, BrdU and the mitotic marker phospho-histone H3 (p-H3). We observed a consistent pattern of proliferative responses to supraphysiological Myc in tissues (FIGS. 1b and c) that fell into three general classes: 1. Adult tissues such as liver, lung and pancreas with normally low levels of endogenous Myc (FIG. 1d) but capable of significant regeneration after injury. Such tissues showed a marked induction of proliferation upon Myc activation. 2. Adult tissues, such as kidney, heart and brain, with normally low levels of endogenous Myc and a limited capacity to regenerate, demonstrated only a negligible rise in proliferation. 3. Tissues with constitutively high proliferative rates and substantial constitutive levels of endogenous Myc (FIG. 1d), such as thymus and spleen, in which activation of ectopic Myc elicited no significant additional proliferation above the already high basal level. Neither sustaining MycER^{T2} activation for three days nor increasing the expression of MycER^{T2} from 1 to 2 copies of R26^{CMER} changed the proliferative responses to Myc in any tissue (FIG. 8e to h). Hence, different tissues harbour different inherent sensitivities to Myc-driven proliferation. Of note, quantification of the broad proliferative marker Ki67 revealed a small but significant increase in some cells within the heart in response to global Myc activation. However, these changes were confined to non-cardiomyocytes.

Target Genes and Tissue Specificity of Myc Binding is Dictated at the Chromatin Level

There are several plausible explanations for the failure of acute Myc activation to induce a proliferative response in tissues such as heart and brain. First, non-responsive tissues might express high levels of one or more of the Mxd proteins, which could directly antagonise Myc's transcriptional function by sequestering Max and/or competing for its binding to shared target genes. To investigate this possibility, we determined the levels of Mxd expression in various tissues. Although expression of Mxd family members varied across tissues, we saw no correlation between the extent of Myc-induced proliferation and the levels of Mxd transcripts. Next, we determined whether Myc is still able to bind its target genes using ChIP-sequencing analysis of Myc binding sites 4 hours after activation in exemplar responsive (liver) and non-responsive (heart) tissue. We observed a dramatic increase in gene occupancy upon MycER^{T2} activation in both tissues, from a few hundred sites in wild-type control, reflecting the low levels of endogenous Myc expression in these tissues to ˜30,000 after Myc activation (FIG. 2a). Hence, the failure of the heart to proliferate in response to Myc activation cannot be attributed to the inability of ectopic Myc to access its target genes. Around a quarter of Myc target binding sites were common to both tissues, with a significant overlap principally at promoter elements (FIGS. 2b and c). Motif analysis demonstrated significant enrichment for the E-box consensus sequences at these regions (FIG. 2d) and gene ontology indicated that these overlapping promoter binding sites located to ‘classic’ core Myc-dependent genes implicated in control of gene expression, mRNA processing and mitotic cell cycle (FIG. 2d). In addition to these common targets, however, Myc also bound discrete sets of promoter elements that differed between the two tissues (FIG. 2b). These tissue-specific sites were also enriched for E-box consensus sequences but demonstrated a generally lower affinity for Myc in comparison to the common core elements (FIGS. 2e and f). Interestingly, these tissue-specific promoter elements were associated with genes whose expression defines each tissue (i.e. hepatocytes versus cardiomyocytes respectively—FIGS. 2e and f), consistent with the idea that Myc binds already active/open promoters. Myc ChIP-seq profiles, RNA Polymerase II (P0111) ChIP-seq in normal liver and heart, ATAC-seq in purified cardiomyocytes, publicly available data for chromatin marks of active transcription (H3K27 acetylation H3K4 tri-methylation and H3K4 mono-methylation) and DNAse-seq in heart and liver all concurred that Myc binding closely overlaps with transcriptionally active promoter and distal elements in each tissue. This holds true across both common and tissue-specific Myc-bound sites. For example, promoters bound in the heart but not liver are characterised by absence of active chromatin marks and PolII binding in the liver. Conversely, promoters bound by Myc only in liver lack active chromatin marks or associated PolII in heart (FIGS. 2g and h). To confirm Myc binding to genes implicated in the transcriptional control of cell cycle in the heart, liver and purified cardiomyocytes, we performed sub analysis of Myc binding and open chromatin marks in mitotic cell cycle gene set (GO: 0000278). Over 60% of these sites were marked as open and Myc bound (FIG. 2i). These data confirm that Myc binds “open” chromatin at both promoters and distal sites (enhancers) and that the disposition of chromatin accessibility is preconfigured in a tissue-specific way. Furthermore, the data indicate that adult terminally differentiated heart retains an open chromatin architecture at sites required for Myc to drive transcriptional control of proliferation and that the regenerative capacity of the heart is lost due to transcriptional control rather than an epigenetic block.

Myc Binding to Target Genes does not Necessarily Correlate with their Efficient Transcription

Since Myc efficiently binds target gene DNA in both heart and liver, we investigated other potential mechanism(s) that might limit proliferative output in the non-responsive heart. We first determined the overall transcriptional output in various tissues (heart, kidney, liver, lung) of R26^{CMER/+} and R26^{+/+} control mice following acute Myc activation for 4 hours. Differentially expressed genes (DEGs) were called in each tissue based on the fold-change (FC) in mRNA levels between R26^{CMER/+} and R26^{+/+} control mice (Log 2FC≥0.5, q≤0.05). In agreement with previous observations (Kress et al., 2015; Sabò et al., 2014; Walz et al., 2014; Zeller et al., 2006), the total number of DEGs in each tissue (FIG. 3a) was much lower than the number of Myc-bound promoters, albeit with significant inclusive overlap (FIG. 3b). Comparison of DEGs in the liver, lung, kidney and heart indicated considerable commonality amongst up-regulated genes (FIG. 3c) implicated in core Myc-regulated processes such as ncRNA, rRNA and tRNA, metabolic processes (FIG. 3d). By contrast, Myc repressed genes showed very little overlap across tissues, although gene ontology analysis did suggest that genes overlapping in 2 or more organs favoured involvement in negative regulation of cell movement, locomotion and migration (FIGS. 3e and f). Tissue-specific genes down-regulated by Myc showed significant overlap with genes normally expressed within that tissue, (FIG. 3g) and their functions were generally associated with differentiated tissue-specific processes (FIG. 3h).

Remarkably, DEG numbers also correlated well with each tissue's proliferative response to MycER^{T2} (FIG. 3a, FIGS. 1b and c): tissues with the lowest overall transcriptional changes displayed the least proliferative responses. However, while activation of ectopic Myc induced significant transcriptional changes to a greater number of target genes in liver than in heart, quantitative analysis of the RNA showed that most of the mRNAs that were induced in liver also exhibited modest, but non-significant, responses in the heart (FIG. 3i). Indeed, gene-set enrichment analysis in the heart confirmed a significant trend in the increased expression of common Myc targets, even when defined as targets commonly upregulated in response to Myc in all tissues excluding the heart (FIG. 3j). Hence, the transcriptional response to activation of ectopic Myc in the heart is not absent but highly attenuated and below that required to initiate cell proliferation. This refractoriness of the heart transcriptome to Myc remained evident even after prolonged (24 hours) Myc activation or in response to very high Myc levels (homozygous R26^{CMER} mice).

Productive Myc Transcriptional Output is Limited by Availability of Transcriptional Cofactors

The most plausible explanation for our data is that the attenuated transcriptional and biological response to Myc in heart is due to either active transcriptional inhibition and/or insufficiency of requisite transcriptional co-factors or machinery. Consistent with this, both the total and phosphorylated levels of the C terminal domain of RNA PolII (Rpb1) were much lower in non-responsive heart and kidney than in Myc-responsive tissues such as the liver and lung (FIG. 4a). Since recruitment of P-TEFb is necessary for transcriptional elongation, we next assessed the expression levels of the components of P-TEFb—CDK9 and CyclinT1. Both CDK9 and CyclinT1 proteins were present at considerably lower levels in the heart and kidney compared with lung or liver, along with components of the pause factors NELF and DSIF (FIGS. 4a and b). Conversely, Larp7, a negative regulator of P-TEFb, was relatively more abundant in the heart and kidney, as was the 7SK short non-coding RNA that serves as the scaffold for the P-TEFb inactivation complex (FIGS. 4a and c). This intimated that the inability of Myc to elicit transcriptional changes in the heart arises from a tissue-specific deficiency in core transcriptional cofactors, such as P-TEFb and RNA PolII itself, which are required for efficient transcriptional initiation and elongation. To determine in vitro if P-TEFb was limiting Myc driven transcription we purified adult cardiomyocytes from R26^{CMER/+}+ mice and overexpressed Ccnt1 with an Adeno virus carrying Ccnt1. Ectopic over-expression of Cyclin T1 in adult R26^{CMER/+} cardiomyocytes in vitro efficiently abrogated their normal refractoriness to Myc and permitted efficient expression of Myc target genes which were previously shown to be unresponsive in the adult heart (Bzw2, Pinx1, Polr3d, St6 and Cdc25a) (FIG. 5a to d). Together, these data support the notion that transcriptional activity of Myc is limited by P-TEFb availability.

Given the finding that Larp7 and the 7SK short non-coding RNA were relatively more abundant in the heart and kidney, this suggests that either or both of these may be alternative targets for therapy. For example, we hypothesise that a Larp7 knockdown (for example, via RNAi or CRISPR) could replace cyclin T1 overexpression. Hence, an aspect of the present invention further provides a method of therapy comprising reducing expression of Larp7 in a subject in need thereof. Suitable CRISPR guide sequences for targeting Larp7 include ATCCGGAGGAAAAAACCTC (SEQ ID NO: 30), AGTCTACTGGAGATCCAAA (SEQ ID NO: 31), AAGAAAGGCCGAATGAAAA (SEQ ID NO: 32), CTTACCTGAAGTCAGAACA (SEQ ID NO: 33), ACTTTTGATCGGGGAGCTA (SEQ ID NO: 34). These may be used in a CRISPR drop-out screen, for example as described in Tzelepis et al, 2016, Cell Reports 17, 1193-1205. The Larp7 amino acid and nucleotide sequences are given as SEQ ID NO: 35 and SEQ ID NO: 36, respectively.

Facilitating Myc-Dependent Transcriptional Activation in Terminally Differentiated Cardiomyocytes In Vivo Restores their Proliferative Capacity

Expression of CDK9, CyclinT1 and RNA PolII all progressively decrease during post-natal cardiac maturation (Sano et al., 2002). We therefore first determined the extent of Myc-dependent transcriptional responsiveness in juvenile heart tissue. Unlike the adult heart, 15-day old juvenile hearts express levels of P-TEFb, RNA PolII comparable to those in adult liver (FIG. 6a and FIGS. 9a and b). Acute (4 hours) MycER^{T2} activation in R26^{CMER/+} juvenile heart induced expression of a panel of common Myc transcriptional target genes (Bzw2, Pinx1, Polr3d, St6 and Cdc25a) normally up-regulated by Myc in the liver but not in adult heart (FIG. 5b). RNA sequencing indicated a proficient global transcriptional response in juvenile heart (1601 DEGs up-regulated and 1133 down-regulated; FIG. 5c), with significant enrichment for common Myc targets (FIG. 9c), defined from the overlap of genes upregulated in response to the activation of Myc in all four tissues. Following 24 hours of tamoxifen treatment, juvenile R26^{CMER/+} mice heart exhibited a marked proliferative response indicated by substantial induction of both G1 (Cdk4) and G2 (Cdk1/Ccnb1) cell cycle genes (FIG. 9d), enrichment of pro-cell cycle progression gene sets (FIG. 9e) and significant levels of mitosis (FIG. 9g) specifically in troponin T-positive cardiomyocytes. The extent of Myc induced cardiomyocyte proliferation was comparable to that observed in tamoxifen-activated R26^{CMER/+} adult liver (FIG. 6d). To confirm that Myc was acting to induce proliferation directly in cardiomyocytes themselves, we restricted expression of MycER^{T2} to cardiomyocytes using the using Myh6Cre allele (Tg(Myh6-cre)2182Mds/J (Agah et al., 1997) in which Cre recombinase is driven from the Myh6 promoter that directs expression of the Myosin heavy chain a isoform solely in cardiomyocytes. Myh6Cre; R26L^{SL-CMER/+} mice were generated and at 15 days of age given a single bolus of tamoxifen to activate MycER^{T2}. Within 48 hrs, Myh6Cre; R26^{LSL-CMER/+} hearts displayed p-H3 and Ki67 positivity specifically in cardiac troponin T and PCM1 positive cardiomyocytes, displayed Aurora B Kinase positivity at centrally located mid-bodies (FIG. 6e and FIG. 9f) and had doubled in size compared with R26^{LSL-CMER/+} controls (FIGS. 6f and g). Overt cell division was evident by a 1.8 fold increase in cardiomyocyte number without any discernible change in cardiomyocyte size (FIGS. 6g and h and FIG. 9h).

To further confirm cytokinesis, transmission electron micrographs (TEM) from Myh6Cre; R26^{LSL-CMER}/+ mice 48 hours post tamoxifen treatment were produced, 3D rendering allowed the identification of cardiomyocytes in mitosis and cytokinesis (FIG. 6i). Over the longer term, transient elevated MycER^{T2} activity in 15 day old Myh6Cre; R26^{LSL-CMER/+} mice caused a large increase in heart size, increased p-H3 immunoreactivity, a large proportion of cardiomyocytes displaying disassembled sarcomeres and mice did not survive beyond 4 days post MycER^{T2} activation (FIG. 9i).

To directly test if increasing P-TEFb activity within cardiomyocytes of the adult heart in vivo is permissive for efficient Myc transcriptional activation and proliferation we infected wild type, R26^{CMER/+}, Myh6Cre; R26^{LSL-CMER/+} and R26^{LSL-CMER/+} mice with an adeno-associated virus directing ectopic expression of Cyclin T1 or RFP specifically in cardiomyocytes under the troponin T promoter. After 4 weeks we confirmed viral Cyclin T1 overexpression in 34% of cardiomyocytes by immunohistochemical analysis (FIG. 7a). As described previously and confirmed here, elevated expression of Ccnt1 in cardiomyocytes leads to an increase in CDK9 and phosphorylated RNA PolII (S2) (FIG. 7b). Acute MycER activation in the presence of overexpressed Cyclin T1 induced the transcription of a panel of common Myc target genes normally up-regulated by Myc in the liver but not in the adult heart (FIG. 7c and FIG. 10a). RNA sequencing indicated a proficient global transcriptional response to the activation of Myc in the presence of ectopic Cyclin T1 in the adult heart or purified cardiomyocytes, with positive enrichment for common Myc targets and pro-cell cycle progression gene sets (FIG. 10b). Positive enrichment of common Myc targets was also observed in cardiomyocytes purified from Cyclin T1 infected Myh6Cre; R26^{LSL-CMER/+} hearts verses uninfected Myh6Cre; R26^{LSL-CMER/+} hearts 4 hours post activation of Myc (FIG. 7d). An increase in the rate of transcription of common Myc targets in cardiomyocytes purified from Cyclin T1 infected Myh6Cre; R26^{LSL-CMER/+} hearts in comparison to Cyclin T1 infected control R26^{LSL-CMER/+} hearts was inferred by a significant increase in the ratio of premature/mature mRNA (FIG. 10c). In contrast, this ratio was unchanged for genes known to be highly expressed in the heart (FIG. 10c), indicating that activation of Myc in the presence ectopic Cyclin T1 does not cause a global amplification of the pre-existing transcriptional repertoire of the adult heart.

A short pulse of MycER^{T2} activation in combination with Cyclin T1 overexpression resulted in a marked proliferative response in hearts collected at 48 hours post a single dose of tamoxifen, cardiomyocytes displaying markers of cell cycle, mitosis (FIGS. 7e and f, FIGS. 10d and e), cytokinesis (FIG. 6e and FIG. 10e), expression of mitosis and cytokinesis gene sets (FIG. 10f), increased heart size (FIG. 7g, h and FIG. 10g). Furthermore, 72 hours post MycER^{T2} activation in combination with Cyclin T1 overexpression led to a further increase in heart size accompanied by an increase in cardiomyocyte number without any change in cardiomyocyte size (FIG. 7g to i) or nucleation (FIG. 10h). A single transient burst of MycER^{T2} activity in Cyclin T1 expressing Myh6Cre; R26^{LSL-CMER/+} adult cardiomyocytes was compatible with long-term survival, with no histological abnormalities detected at 28 days after MycER^{T2} activation (FIG. 10i). Collectively, these data illustrate that Myc transcriptional activity can be facilitated in adult cardiomyoctes by co-activation of the transcriptional co-factor P-TEFb thus enabling adult heart tissue to re-enter the cell cycle.

Furthermore, constitutively elevated levels of P-TEFb have been noted in the hearts of TetO-HRas; Myh6-tTA (Tg(tetO-HRAS)65Lc/Nci); Myh6tTA (Tg(Myh6-tTA)6Smbf/Jm) mice, which constitutively express the oncogenic HRas^{G12V} protein in myocardium from weaning age. To test the prediction that such augmented basal levels of p-TEFb render heart tissue Myc responsive, these mice were crossed into the R26^{CMER/+} switchable Myc background to generate TetO-HRas; Myh6-tTA; R26^{CMER/+} mice. Doxycycline was withdrawn from TetO-HRas; Myh6-tTA; R26^{CMER/+} animals at weaning to induce expression of HRas^{G12V} and at adulthood animals exhibited the reported elevated levels of CyclinT1, RNA PolII and phospho-RNA PolII (S2) (FIG. 11i). Activation of MycER^{T2} (for 24 hours) in these animals induced multiple cell cycle genes (Cdk4, Ccnd1, Cdk1, Ccnb1) (FIG. 11e) not induced by Myc in control TetO-HRas; Myh6-tTA; R26+^{+/+} hearts and a marked proliferative response, mitosis (17.8% of cardiomyocytes phospho-histone H3 positive) and cytokinesis (FIG. 11a to d). We also observed Myc-dependent modulation of a broad panel of Myc target genes that were not affected in control adult heart (FIG. 11f). RNA sequencing confirmed increased transcription (976 DEGs up-regulated and 663 down regulated; FIG. 11g) of Myc target genes whose identities overlapped with genes induced by MycER^{T2} in adult liver, lung and kidney. To determine the extent that the transcriptional changes in TetO-HRas; Myh6-tTA; R26^{CMER/+} and TetO-HRas; Myh6-tTA; R26^{+/+} hearts revert to a neonatal (P1) proliferative state we compared the transcriptional profile to cardiomyocytes isolated from surgically infarcted hearts at different stages of development. Our unbiased approach revealed that the P1 neonatal regeneration signature genes were markedly enriched and correlated most closely with TetO-HRas; Myh6-tTA; R26^{CMER/+} hearts but not with cardiomyocytes isolated from adult infarcted hearts or with control TetO-HRas; Myh6-tTA; R26^{+/+} (no Myc) hearts (FIG. 11h). Collectively, these data illustrate that Myc transcription can be driven in adult cardiomyoctes by co-expression of the transcriptional co-factor P-TEFb thus enabling adult heart tissue to re-enter the cell cycle.

### DISCUSSION

Myc is widely acknowledged to play a pivotal coordinating role in the transcriptional control of somatic cell proliferation and tissue regeneration and its activity is deregulated and typically elevated in the majority of aggressive cancers (Amati et al., 1993; Eilers et al., 1991; Roussel et al., 1991). Past studies have largely focused on identifying Myc transcriptional programmes that are common across cell types. However, Myc exhibits great cell type variability, both with respect to how efficiently it can drive cell proliferation and the phenotypes its activities elicit in different tissues (Kortlever et al., 2017; Pelengaris et al., 2002, 1999; Shchors et al., 2006). In addition, previous studies have been limited by an inevitable focus on the longer-term outcomes of Myc activation, which makes it very difficult to parse the direct impact of Myc from the indirect pleiotropic consequences of the primary programmes that Myc engages across different tissues. To contrast and compare the direct, initial impact of Myc activation at oncogenic levels on different tissues, we developed a unique mouse model whereby the Myc encoding sequence is inserted downstream of the endogenous Rosa26 promoter, which is expressed at comparable levels across all tested tissues. This model is enhanced in several ways: first, by the option of a variant, augmented Rosa26 promoter incorporating additional CAG (CMV and β-actin promoter elements) that increases the expression level of Myc to supraphysiological levels frequent in many cancers, thereby establishing an ascending allelic series (R26^{MER/+}, R26^{MER/MER}, R26^{CMER/+}, R26^{CMER/MER}, R26^{CMER/CMER}) of increasing Myc levels with which to ascertain the relative oncogenic roles of Myc deregulation versus over-expression. Unlike most tissue-specific promoters used to drive transgenic oncogenes, neither Rosa26 nor Rosa CAG promoters are repressed by Myc activation, obviating a major confounding issue in most conventional transgenic models. Second, replacing Myc with the well-validated, reversibly switchable 4-OHT-dependent MycER^{T2} variant allows the analysis of the immediate and direct impact of Myc on its target genome through rapid and synchronous MycER^{T2} activation by 4-OHT. Third, inclusion of tissue-specific regulation via a Cre recombinase-excisable transcriptional STOP element provides the option for tissue restricted MycER^{T2} expression.

In our current studies, we have used the egg-specific ZP3 promoter to engage ubiquitous expression of MycER^{T2} in R26^{CMER} mice and used this to compare the direct impacts of oncogenic Myc activity in different adult mouse tissues. Using proliferation as the phenotypic signature of Myc output, we identified three general classes of response to activation of oncogenic levels of Myc: tissues that proliferated in response to MycER^{T2} activation; tissues that did not; and tissues that exhibited innately high endogenous Myc and proliferative indices. These innate tissue responses were maintained even when higher levels of Myc were expressed over an extended period of time. Organs with inherently high levels of cell turnover supplied from a dedicated stem cell compartment, such as the intestine and hematopoietic system, exhibit constitutively high endogenous Myc levels and activation of ectopic MycER^{T2} only modestly augments their already high proliferation ability. Tissues such as the liver, lung and pancreas are normally quiescent, have low endogenous levels of Myc, but capable of facultative regeneration in response to injury (Baddour et al., 2012). Activation of ectopic MycER^{T2} in such tissues induces a profound proliferative response. Finally, adult tissues such as the brain and heart have low endogenous levels of Myc, have little or no regenerative potential, regenerate poorly or not at all after injury and generally form scar tissue, and show no proliferative response to MycER^{T2} activation. The correlation between proliferative response to ectopic Myc in these tissues (FIGS. 1b and c) and the innate regenerative potential of each tissue (reviewed in Kotton and Morrisey, 2014) is striking and suggests an underlying mechanistic connection.

Myc shares both its obligate dimerization partner Max and its canonical E-box DNA binding specificity with the Mxd family of transcriptional repressor proteins and it has been suggested that Myc and Mxd compete for Max and thereby operate within a mutually antagonistic network (Ayer et al., 1993; Kretzner et al., 1992). However, we found no correlation across multiple tissues between levels of Mxd expression and proliferative response to Myc—indeed, Mxd proteins are more highly expressed in some Myc-responsive tissues cells (pancreas, liver, and lung). Furthermore, our data indicating high levels of Mxd expression in proliferating tissues such as spleen and thymus shows that, as previously suggested (Baudino and Cleveland, 2001), Mxd proteins can co-exist intracellularly with Myc without necessarily antagonising Myc action. The different patterns of chromatin binding that Max-Mxd and Myc-Max dimers exhibit, possibly arising from distinct residues in the Mxd versus Myc DNA-binding basic regions, also suggest that the two classes of transcription factor operate largely independently of each other (O'Hagan et al., 2000; Solomon et al., 1993). These data confirm that, as previously suggested (Baudino and Cleveland, 2001), Mxd proteins in various organs do not significantly antagonise Myc activity.

ChIP-seq analysis 4 hours post MycER^{T2} activation in exemplar Myc-responsive versus non-responsive tissues (respectively, liver and heart) showed a dramatic increase in gene occupancy in both tissues. Hence, tissue non-responsiveness to Myc is not due to failure of ectopic Myc to access its chromatin targets. Indeed, we found striking similarities between the numbers of sites to which Myc is bound across the different tissues analysed, underscoring that the phenotypic outcome effected by Myc is not solely dictated at the level of recruitment to chromatin.

The significant overlap in Myc-bound promoter regions across both the liver and heart affirms the widely held notion that Myc binds a set of target genes common to multiple cell types and implicated in governing core processes required for cell growth and cell cycle progression. However, Myc also binds a set of promoter elements specific to each tissue, indicating that the Myc-dependent cistrome is cell type-specific. We also note that this tissue-specific Myc binding repertoire is characterised by the presence of chromatin marks indicative of open chromatin, in keeping with the findings of others. Interestingly as early as 4-hours post Myc activation, we clearly see a significant number of genes to be transcriptionally repressed. These repressed genes principally articulate differentiated cell-type functions and show very little overlap between tissue types. Moreover, around a third (34%) of the repressed genes appear to be indirect targets since Myc is not detected at their promoter elements (FIG. 3b). Suppression of differentiation-specific genes by oncogenic Myc has been widely observed irrespective of cell or tissue type and is an example of the robust inverse generic relationship between cell proliferation and differentiation (Ruijtenberg and van den Heuvel, 2016).

A simple hypothetical explanation for the pervasive suppression of differentiation by Myc is that certain components of transcriptional machinery, such as P-TEFb or even total levels of RBP1, available for loading onto promoters are limiting, in which case repression of differentiation-specific genes is simply a consequence of their redeployment to Myc target genes, a mechanism recently proposed in 3T3 fibroblasts (De Pretis et al., 2017). Analogous scarcities in transcriptional machinery could also explain why some tissues fail to respond transcriptionally to Myc. Myc has been shown to modify both RNA PolII loading and its phosphorylation by P-TEFb (De Pretis et al., 2017; Eberhardy and Farnham, 2002; Jonkers and Lis, 2015; Kanazawa et al., 2003; Rahl et al., 2010; Shao and Zeitlinger, 2017). Prompted by the recent observation that MYC overexpression is the key molecular determinant dictating sensitivity to CDK9 inhibition in hepatocellular carcinoma (Huang et al., 2014), we showed that responsiveness of individual tissues to Myc correlates with the levels of expression of the basal transcription factors P-TEFb and RNA PolII, the pause factors, DSIF and NELF, and, inversely, with the expression of the P-TEFb repression complex.

Adult mammalian heart is a prototypical, terminally-differentiated, Myc non-responsive tissue in which Cyclin T1 is known to be a limiting transcriptional regulator whose chronic ectopic overexpression elicits promiscuous RNA polymerase activity and cardiac hypertrophy. While P1 neonatal heart retains proliferative potential, this rapidly declines after birth and is largely lost by P7. This decline in neonatal cardiac proliferative potential correlates with down-regulation of multiple genes involved in cell cycle including endogenous Myc, which is high at P1 but significantly reduced by P5 (FIG. 9a). Although changes in microRNAs, modulation of the Hippo pathway, and wholesale metabolic switching from glycolysis to oxidative phosphorylation have all been implicated, the mechanism underlying post-natal cardiac maturation and loss of regenerative potential remains unknown.

Interestingly, both the neonatal mouse heart and the zebrafish larval heart express high levels of P-TEFb. Whereas, the zebrafish adult heart retains 70% of the larval levels of P-TEFb and the ability to regenerate throughout development, P-TEFb levels in the adult mouse heart drop to 15%. Little is known regarding the regulation of P-TEFb during mammalian cardiac development. Levels of Cyclin T1 are regulated at the transcriptional and post-transcriptional level, with mitogens and cytokine signaling known to increase Cyclin T1 protein stability. We and others have shown that the level of Cyclin T1 is the key factor regulating the level and activity of P-TEFb within cardiomyocytes. As shown in FIG. 11, oncogenic Ras signaling increases Cyclin T1 in the heart, leading to cardiac hypertrophy. It is tempting to speculate that the noted oncogenic cooperation between Ras and Myc may, in part, rely on a Ras-dependent increase in P-TEFb, promoting Myc-driven transcription.

Re-establishing regenerative proliferation in the adult heart has proven very difficult. Inhibition of Hippo signalling, enforced expression of both G1 (CDK4 and Ccnd1) and G2/M (CDK1 and Ccnb1) factors, and hypoxia all induce modest regeneration but at levels well below those seen in P1 neonatal heart. While the adult heart is refractory to Myc induced proliferation, enforced ectopic Myc expression extended the window of neonatal cardiomyocyte proliferation, up to around P15, a time-point that correlates with high endogenous cardiac P-TEFb expression. The large increase in cardiomyocyte number and Aurora B localization to centrally located mid-bodies suggests that cardiomyocytes isolated from juvenile mice can progress through the cell cycle and many can complete cytokinesis. Remarkably, we show that an increase in the level of P-TEFb activity in the adult heart is sufficient to support ectopic Myc dependent transcription, proliferation and cytokinesis of cardiomyocytes in vivo, resulting in increased heart size and cardiomyocyte number within a very short timeframe. Importantly this transient wave of proliferation induced in a smaller fraction of total cardiomyocytes than in juvenile mice is compatible with long-term survival. It will be of great interest to determine if this genetic combination will prove beneficial in models of cardiac injury.

Despite the significant changes in regenerative capacity and responsiveness to Myc that attend cardiac development from embryonic stem cells through mesoderm to precursor cardiomyocyte to cardiomyocyte, the chromatin status at promoters for genes involved in basic cellular function, including cell cycle regulation, show very little variation (Heintzman et al., 2009; Wamstad et al., 2012). Similarly, the Myc binding sites of the adult liver (proliferative) and the adult heart (non-proliferative) exhibit significant overlap, indicating that even after its maturation, adult, terminally-differentiated heart retains an open chromatin architecture at sites required to drive transcriptional control of proliferation. Hence, if Myc could be induced by mitogenic stimulus in the heart, cardiac epigenome architecture does not preclude heart regenerative capacity: rather, it is the inability of Myc to drive transcriptional output from regenerative target genes (due to lack of the P-TEFb/RNA Pol II complex) that thwarts cardiomyocyte proliferation.

Overall, our data indicate that tissue regenerative capacity is tightly linked to the capacity of that tissue to respond to Myc and that tissue Myc responsiveness is governed principally by availability of key components of the core transcriptional machinery, which Myc co-opts to drive its regenerative biological output. However, in addition to its core target genes, which are common across tissues, a significant number of its targets are expressed in a tissue-specific manner. By contrast, tissue-specific accessibility of target genes to Myc appears to be dictated principally by hard-wired, tissue-specific epigenome organization. Our current study focuses in the main on liver and heart as, respectively, prototypical examples of regenerative and Myc responsive versus non-regenerative and Myc-unresponsive tissues.

### Example II

The adult mouse brain displays many of the same non-regenerative features as the adult mouse heart, such as increased cell size, a switch from glycolysis to oxidative phosphorylation and increased mitochondrial mass (Agathocleous et al., 2012; Zheng et al., 2016). Likewise, the nephron cells of the kidney have a huge metabolic demand, second only to the heart. We determined the level of Myc and Ccnt1/Cdk9 proteins throughout the mouse post-natal development. Similar to the heart, the level of all 3 of these proteins drop with age to adulthood, indicating P-TEFb levels may be limiting Myc driven transcription in adulthood (FIG. 12a). MycER was activated in 10 day old mice with a single injection of tamoxifen, immunohistochemical staining showed some increased positivity of p-H3 in MycER expressing brain and kidney tissues (FIG. 12b). Therefore, enforced ectopic Myc expression can extended the window of neonatal proliferation, up to around P15, a time-point that correlates with high endogenous cardiac P-TEFb expression, in both the brain and kidney.

### REFERENCES

Agah, R. et al., 1997. Gene recombination in postmitotic cells: Targeted expression of Cre recombinase provokes cardiac-restricted, site-specific rearrangement in adult ventricular muscle in vivo. J. Clin. Invest.
Agathocleous, M. et al. Metabolic differentiation in the embryonic retina. Nat. Cell Biol. 14, (2012).
Akalin, A. et al., 2015. Genomation: A toolkit to summarize, annotate and visualize genomic intervals. Bioinformatics 31, 1127-1129.
Amati, B. et al., 1992. Transcriptional activation by the human c-Myc oncoprotein in yeast requires interaction with Max. Nature 359, 423-426.
Amati, B. et al., 1993. The c-Myc protein induces cell cycle progression and apoptosis through dimerization with Max. EMBO J. 12, 5083-5087.
Anders, S. et al., 2015. HTSeq-A Python framework to work with high-throughput sequencing data. Bioinformatics 31, 166-169.
Ayer, D. E. et al., 1993. Mad: A heterodimeric partner for Max that antagonizes Myc transcriptional activity. Cell 72, 211-222.
Baddour, J. A. et al., 2012. Organ repair and regeneration: An overview. Birth Defects Res. Part C—Embryo Today Rev.
Bailey, T. L. et al., 2012. Inferring direct DNA binding from ChIP-seq. Nucleic Acids Res. 40, 1-10.
Barboric, M. et al., 2005. Interplay between 7SK snRNA and oppositely charged regions in HEXIM1 direct the inhibition. EMBO J. 24, 4291-4303.
Baudino, T. A. et al., 2001. The Max Network Gone Mad. Mol. Cell. Biol. 21, 691-702.
Bianchi, V. et al., 2016. Integrated Systems for NGS Data Management and Analysis: Open Issues and Available Solutions. Front. Genet. 7, 1-8.
Blackwood, E. M, et al., 1991. Max: A helix-loop-helix zipper protein that forms a sequence-specific DNA-binding complex with Myc. Science (80-.). 251, 1211-1217.
Blackwood, E. M. et al., 1992. Myc and Max associate in vivo. Genes Dev. 6, 71-80.
Blecher-Gonen, R. et al., 2013. High-throughput chromatin immunoprecipitation for genome-wide mapping of in vivo protein-DNA interactions and epigenomic states. Nat. Protoc. 8, 539-554.
Bolger, A. M. et al., 2014. Trimmomatic: A flexible trimmer for Illumina sequence data. Bioinformatics 30, 2114-2120.
Bouchard, C. et al., 2001. Regulation of cyclin D2 gene expression by the Myc/Max/Mad network: Myc-dependent TRRAP recruitment and histone acetylation at the cyclin D2 promoter. Genes Dev. 15, 2042-2047.
Chen, E. Y. et al., 2013. Enrichr: Interactive and collaborative HTML5 gene list enrichment analysis tool. BMC Bioinformatics 14.
Dang, C. V, 2013. MYC, metabolism, cell growth, and tumorigenesis. Cold Spring Harb. Perspect. Med. 3, a014217.
De Pretis, S. et al. Gene expression INSPEcT: a computational tool to infer mRNA synthesis, processing and degradation dynamics from RNA- and 4sU-seq time course experiments. Bioinformatics 31, 2829-2835 (2015).
De Pretis, S. et al., 2017. Integrative analysis of RNA polymerase II and transcriptional dynamics upon MYC activation. Genome Res. 10, 1658-1664.
De Vries, W. N. et al., 2000. Expression of Cre Recombinase in Mouse Oocytes: A Means to Study Maternal Effect Genes. Genesis 26, 110-112.
Diolaiti, D. et al., 2015. Functional interactions among members of the MAX and MLX transcriptional network during oncogenesis. Biochim. Biophys. Acta—Gene Regul. Mech. 1849, 484-500.
Dobin, A. et al., 2013. STAR: Ultrafast universal RNA-seq aligner. Bioinformatics 29, 15-21.
Dunham, I., et al. An integrated encyclopedia of DNA elements in the human genome. Nature 489, 57-74.
Eberhardy, S. R. et al., 2002. Myc recruits P-TEFb to mediate the final step in the transcriptional activation of the cad promoter. J. Biol. Chem. 277, 40156-40162.
Eilers, M. et al., 1991. The MYC protein activates transcription of the alpha-prothymosin gene. EMBO J. 10, 133-141.
Evan, G. I. et al., 1992. Induction of apoptosis in fibroblasts by c-myc protein. Cell 69, 119-128.
Furlan, M. et al. Genome-wide dynamics of RNA synthesis, processing and degradation without RNA metabolic labeling. bioRxiv Prepr. 1-26 (2019).
Gamper, I. et al., 2017. Determination of the physiological and pathological roles of E2F3 in adult tissues. Sci. Rep. 7.
Gentleman, R. et al., 2004. Bioconductor: open software development for computational biology and bioinformatics. Genome Biol. 5, R80.
Gilsbach, R. et al., 2014. Dynamic DNA methylation orchestrates cardiomyocyte development, maturation and disease. Nat. Commun. 5, 5288.
He, N. et al., 2008. Article A La-Related Protein Modulates 7SK snRNP Integrity to Suppress P-TEFb-Dependent Transcriptional Elongation and Tumorigenesis. Mol. Cell 29, 588-599.
Heintzman, N. D. et al., 2009. Histone modifications at human enhancers reflect global cell-type-specific gene expression. Nature 459, 108-112.
Huang, C. H. et al., 2014. CDK9-mediated transcription elongation is required for MYC addiction in hepatocellular carcinoma. Genes Dev. 28, 1800-1814.
Huang, D. W. et al., 2009. Systematic and integrative analysis of large gene lists using DAVID bioinformatics resources. Nat. Protoc. 4, 44-57.
Jonkers, I., Lis, J. T., 2015. Getting up to speed with transcription elongation by RNA polymerase II. Nat. Rev. Mol. Cell Biol.
Kanazawa, S, et al., 2003. c-Myc recruits P-TEFb for transcription, cellular proliferation and apoptosis. Oncogene 22, 5707-5711.
Kishore, K. et al., 2015. methylPipe and compEpiTools: A suite of R packages for the integrative analysis of epigenomics data. BMC Bioinformatics 16, 1-11.
Kondrat, J et al. Synthesis of modified mRNA for myocardial delivery; Cardiac Gene Therapy, Vol. 152. p 127-137; 2017.
Kortlever, R. M. et al., 2017. Myc Cooperates with Ras by Programming Inflammation and Immune Suppression. Cell 171, 1301-1315.e14.
Kotton, D. N., Morrisey, E. E., 2014. Lung regeneration: Mechanisms, applications and emerging stem cell populations. Nat. Med.
Kress, T. R. et al., 2016. Identification of MYC-dependent transcriptional programs in oncogene-addicted liver tumors. Cancer Res. 76, 3463-3472.
Kress, T. R. et al., 2015. MYC: Connecting selective transcriptional control to global RNA production. Nat. Rev. Cancer.
Kretzner, L. et al., 1992. Transcriptional activities of the Myc and Max proteins in mammalian cells. Curr. Top. Microbiol. Immunol. 182, 435-443.
Leach, J. P, et al., 2017. Hippo pathway deficiency reverses systolic heart failure after infarction. Nature 550, 260-264.
Li, B., Dewey, C. N., 2011. RSEM: Accurate transcript quantification from RNA-Seq data with or without a reference genome. BMC Bioinformatics 12.
Li, F. et al., 1996. Rapid transition of cardiac myocytes from hyperplasia to hypertrophy during postnatal development. J. Mol. Cell. Cardiol. 28, 1737-1746.
Li, H., 2013. Aligning sequence reads, clone sequences and assembly contigs with BWA-MEM 00, 1-3.
Love, M. I. et al., 2014. Moderated estimation of fold change and dispersion for RNA-seq data with DESeq2. Genome Biol. 15, 1-21.
Martin, M., 2011. Cutadapt removes adapter sequences from high-throughput sequencing reads. EMBnet. journal 17, 10.
McMahon, S. B. et al., 1998. The novel ATM-related protein TRRAP is an essential cofactor for the c-Myc and E2F oncoproteins. Cell 94, 363-374.
Mohamed, T., et al., 2018. Regulation of Cell Cycle to Stimulate Adult Cardiomyocyte Proliferation and Cardiac Regeneration. Cell 173, 104-116.
Murphy, D. J., et al., 2008. Distinct Thresholds Govern Myc's Biological Output In Vivo. Cancer Cell 14, 447-457.
Muzumdar, M. D., et al., 2007. A global double-fluorescent cre reporter mouse. Genesis 45, 593-605.
Nakada, Y., et al., 2017. Hypoxia induces heart regeneration in adult mice. Nature 541, 222-227.
O'Hagan, R. C., et al., 2000. Gene-target recognition among members of the Myc superfamily and implications for oncogenesis. Nat. Genet. 24, 113-119.
Oliveros, J. C., 2007. Venny. An interactive tool for comparing lists with Venn's diagrams.
Pelengaris, S., et al., 2002. Suppression of Myc-induced apoptosis in β cells exposes multiple oncogenic properties of Myc and triggers carcinogenic progression. Cell 109, 321-334.
Pelengaris, S., et al., 1999. Reversible activation of c-Myc in skin: Induction of a complex neoplastic phenotype by a single oncogenic lesion. Mol. Cell 3, 565-577.
Peterlin, B. M., Price, D. H., 2006. Controlling the Elongation Phase of Transcription with P-TEFb. Mol. Cell 23, 297-305.
Porrello, E. R., et al., 2011. Transient Regenerative Potential of the Neonatal Mouse Heart. Science (80-.). 331, 1078-1080.
Porrello, E. R., Olson, E. N., 2014. A neonatal blueprint for cardiac regeneration. Stem Cell Res. 13, 556-570.
Rahl, P. B., et al., 2010. C-Myc regulates transcriptional pause release. Cell 141, 432-445.
Robinson, M. D., et al., 2009. edge R: A Bioconductor package for differential expression analysis of digital gene expression data. Bioinformatics 26, 139-140.
Robinson, M. D., Oshlack, A., 2010. A scaling normalization method for differential expression analysis of RNA-seq data. Genome Biol. 11.
Roussel, M. F., et al., 1991. Myc rescue of a mutant CSF-1 receptor impaired in mitogenic signalling. Lett. To Nat. 353, 361-363.
Ruijtenberg, S., van den Heuvel, S., 2016. Coordinating cell proliferation and differentiation: Antagonism between cell cycle regulators and cell type-specific gene expression. Cell Cycle 15, 196-212.
Sabò, A., et al., 2014. Selective transcriptional regulation by Myc in cellular growth control and lymphomagenesis. Nature.
Sano, M., et al., 2002. Activation and function of cyclin T-Cdk9 (positive transcription elongation factor-b) in cardiac muscle-cell hypertrophy. Nat. Med. 8, 1310-1317.
Shao, W., Zeitlinger, J., 2017. Paused RNA polymerase II inhibits new transcriptional initiation. Nat. Genet.
Shchors, K., et al., 2006. The Myc-dependent angiogenic switch in tumors is mediated by interleukin 1β. Genes Dev. 20, 2527-2538.
Sodir, N. M., et al., 2011. Endogenous Myc maintains the tumor microenvironment. Genes Dev. 25, 907-916.
Solomon, D. L. C., et al., 1993. Distinct DNA binding preferences for the c-Myc/Max and Max/Max dimers. Nucleic Acids Res. 21, 5372-5376.
Walz, S., et al., 2014. Activation and repression by oncogenic MYC shape tumour-specific gene expression profiles. Nature 511, 483-487.
Wamstad, J. A., et al., 2012. Dynamic and coordinated epigenetic regulation of developmental transitions in the cardiac lineage. Cell 151, 206-220.
Wei, B.-R., et al., 2011. TRE-RasG12V 2011 Comp Med. Comp. Med. 61, 109-118.
Wilson, C. H., et al., 2014. The kinetics of ER fusion protein activation in vivo. Oncogene 33, 4877-4880.
Yik, J. H. N., et al., 2003. Inhibition of P-TEFb (CDK9/Cyclin T) Kinase and RNA Polymerase II Transcription by the Coordinated Actions of HEXIM1 and 7SK snRNA 12, 971-982.
Zeller, K. I., et al., 2006. Global mapping of c-Myc binding sites and target gene networks in human B cells. Proc. Natl. Acad. Sci. U.S.A 103, 17834-9.
Zhang, Y., et al., 2008. Model-based analysis of ChIP-Seq (MACS). Genome Biol. 9, R137.
Zhou, Q., et al., 2012. RNA Polymerase II Elongation Control. Annu. Rev. Biochem 81, 119-43.
Zhou, Q., Yik, J. H. N., 2006. The Yin and Yang of P-TEFb Regulation: Implications for Human Immunodeficiency Virus Gene Expression and Global Control of Cell Growth and Differentiation. Microbiol. Mol. Biol. Rev.
Zheng, X. et al. Metabolic reprogramming during neuronal differentiation from aerobic glycolysis to neuronal oxidative phosphorylation. Elife 1-25 (2016). doi: 10.7554/eLife. 13374

SEQUENCE LISTING
SEQ ID NO: 1: MycER^{T2} amino acid sequence
Met P L N V S F T N R N Y D L D Y D S V Q P Y F Y
C D E E E N F Y Q Q Q Q Q S E L Q P P A P S E D I
W K K F E L L P T P P L S P S R R S G L C S P S Y
V A V T P F S L R G D N D G G G G S F S T A D Q L
E Met V T E L L G G D Met V N Q S F I C D P D D E
T F I K N I I I Q D C Met W S G F S A A A K L V S
E K L A S Y Q A A R K D S G S P N P A R G H S V C
S T S S L Y L Q D L S A A A S E C I D P S V V F P
Y P L N D S S S P K S C A S Q D S S A F S P S S D
S L L S S T E S S P Q G S P E P L V L H E E T P P
T T S S D S E E E Q E D E E E I D V V S V E K R Q
A P G K R S E S G S P S A G G H S K P P H S P L V
L K R C H V S T H Q H N Y A A P P S T R K D Y P A
A K R V K L D S V R V L R Q I S N N R K C T S P R
S S D T E E N V K R R T H N V L E R Q R R N E L K
R S F F A L R D Q I P E L E N N E K A P K V V I L
K K A T A Y I L S V Q A E E Q K L I S E E D L L R
K R R E Q L K H K L E Q L R D P G S A G D Met R A
A N L W P S P L Met I K R S K K N S L A L S L T A
D Q Met V S A L L D A E P P I L Y S E Y D P T R P
F S E A S Met Met G L L T N L A D R E L V H Met I
N W A K R V P G F V D L T L H D Q V H L L E C A W
L E I L Met I G L V W R S Met E H P V K L L F A P
N L L L D R N Q G K C V E G Met V E I F D Met L L
A T S S R F R Met Met N L Q G E E F V C L K S I I
L L N S G V Y T F L S S T L K S L E E K D H I H R
V L D K I T D T L I H L Met A K A G L T L Q Q Q H
Q R L A Q L L L I L S H I R H Met S N K G Met E H
L Y S Met K C K N V V P L Y D L L L E A A D A H R
L H A P T S R G G A S V E E T D Q S H L A T A G S
T S S H S L Q K Y Y I T G E A E G F P A T V Stop
Q
SEQ ID NO: 2: MycER^{T2} nucleic acid sequence
ATGCCCCTCAACGTTAGCTTCACCAACAGGAACTATGACCTCGACTACGA
CTCGGTGCAGCCGTATTTCTACTGCGACGAGGAGGAGAACTTCTACCAGC
AGCAGCAGCAGAGCGAGCTGCAGCCCCCGGCGCCCAGCGAGGATATCTGG
AAGAAATTCGAGCTGCTGCCCACCCCGCCCCTGTCCCCTAGCCGCCGCTC
CGGGCTCTGCTCGCCCTCCTACGTTGCGGTCACACCCTTCTCCCTTCGGG
GAGACAACGACGGCGGTGGCGGGAGCTTCTCCACGGCCGACCAGCTGGAG
ATGGTGACCGAGCTGCTGGGAGGAGACATGGTGAACCAGAGTTTCATCTG
CGACCCGGACGACGAGACCTTCATCAAAAACATCATCATCCAGGACTGTA
TGTGGAGCGGCTTCTCGGCCGCCGCCAAGCTCGTCTCAGAGAAGCTGGCC
TCCTACCAGGCTGCGCGCAAAGACAGCGGCAGCCCGAACCCCGCCCGCGG
CCACAGCGTCTGCTCCACCTCCAGCTTGTACCTGCAGGATCTGAGCGCCG
CCGCCTCAGAGTGCATCGACCCCTCGGTGGTCTTCCCCTACCCTCTCAAC
GACAGCAGCTCGCCCAAGTCCTGCGCCTCGCAAGACTCCAGCGCCTTCTC
TCCGTCCTCGGATTCTCTGCTCTCCTCGACGGAGTCCTCCCCGCAGGGCA
GCCCCGAGCCCCTGGTGCTCCATGAGGAGACACCGCCCACCACCAGCAGC
GACTCTGAGGAGGAACAAGAAGATGAGGAAGAAATCGATGTTGTTTCTGT
GGAAAAGAGGCAGGCTCCTGGCAAAAGGTCAGAGTCTGGATCACCTTCTG
CTGGAGGCCACAGCAAACCTCCTCACAGCCCACTGGTCCTCAAGAGGTGC
CACGTCTCCACACATCAGCACAACTACGCAGCGCCTCCCTCCACTCGGAA
GGACTATCCTGCTGCCAAGAGGGTCAAGTTGGACAGTGTCAGAGTCCTGA
GACAGATCAGCAACAACCGAAAATGCACCAGCCCCAGGTCCTCGGACACC
GAGGAGAATGTCAAGAGGCGAACACACAACGTCTTGGAGCGCCAGAGGAG
GAACGAGCTAAAACGGAGCTTTTTTGCCCTGCGTGACCAGATCCCGGAGT
TGGAAAACAATGAAAAGGCCCCCAAGGTAGTTATCCTTAAAAAAGCCACA
GCATACATCCTGTCCGTCCAAGCAGAGGAGCAAAAGCTCATTTCTGAAGA
GGACTTGTTGCGGAAACGACGAGAACAGTTGAAACACAAACTTGAACAGC
TACGGGATCCGGGGTCTGCTGGAGACATGAGAGCTGCCAACCTTTGGCCA
AGCCCGCTCATGATCAAACGCTCTAAGAAGAACAGCCTGGCCTTGTCCCT
GACGGCCGACCAGATGGTCAGTGCCTTGTTGGATGCTGAGCCCCCCATAC
TCTATTCCGAGTATGATCCTACCAGACCCTTCAGTGAAGCTTCGATGATG
GGCTTACTGACCAACCTGGCAGACAGGGAGCTGGTTCACATGATCAACTG
GGCGAAGAGGGTGCCAGGCTTTGTGGATTTGACCCTCCATGATCAGGTCC
ACCTTCTAGAATGTGCCTGGCTAGAGATCCTGATGATTGGTCTCGTCTGG
CGCTCCATGGAGCACCCAGTGAAGCTACTGTTTGCTCCTAACTTGCTCTT
GGACAGGAACCAGGGAAAATGTGTAGAGGGCATGGTGGAGATCTTCGACA
TGCTGCTGGCTACATCATCTCGGTTCCGCATGATGAATCTGCAGGGAGAG
GAGTTTGTGTGCCTCAAATCTATTATTTTGCTTAATTCTGGAGTGTACAC
ATTTCTGTCCAGCACCCTGAAGTCTCTGGAAGAGAAGGACCATATCCACC
GAGTCCTGGACAAGATCACAGACACTTTGATCCACCTGATGGCCAAGGCA
GGCCTGACCCTGCAGCAGCAGCACCAGCGGCTGGCCCAGCTCCTCCTCAT
CCTCTCCCACATCAGGCACATGAGTAACAAAGGCATGGAGCATCTGTACA
GCATGAAGTGCAAGAACGTGGTGCCCCTCTATGACCTGCTGCTGGAGGCG
GCGGACGCCCACCGCCTACATGCGCCCACTAGCCGTGGAGGGGCATCCGT
GGAGGAGACGGACCAAAGCCACTTGGCCACTGCGGGCTCTACTTCATCGC
ATTCCTTGCAAAAGTATTACATCACGGGGGAGGCAGAGGGTTTCCCTGCC
ACAGTCTGACAATT
SEQ ID NO: 3: CAG promoter
CGGCCGCCCACCCTCCCCTTCCTCTGGGGGAGTCGTTTTACCCGCCGCCG
GCCGGGCCTCGTCGTCTGATTGGCTCTCGGGGCCCAGAAAACTGGCCCTT
GCCATTGGCTCGTGTTCGTGCAAGTTGAGTCCATCCGCCGGCCAGCGGGG
GCGGC::G:A:G:GAGGCGCTCCCAGGTTCCGGCCCTCCCCTCGGCCCCG
CGCCGCAGAGTCTGGCCGCGCGCCCCTGCGCAACGTGGCAGGAAGCGCGC
GCTGGGGGCGGGGACGGGCAGTAGGGCTGAGCGGCTGCGGGGCGGGTGCA
AGCACGTTTCCGACTTGAGTTGCCTCAAGAGGGGCGTGCTGAGCCAGACC
TCCATCGCGCACTCCGGGGAGTGGAGGGAAGGAGCGAGGGCTCAGTTGGG
CTGTTTTGGAGGCAGGAAGCACTTGCTCTCCCAAAGTCGCTCTGAGTTGT
TATCAGTAAGGGAGCTGCAGTGGAGTAGGCGGGGAGAAGGCCGCACCCTT
CTCCGGAGGGGGGAGGGGAGTGTTGCAATACCTTTCTGGGAGTTCTCTGC
TGCCTCCTGGCTTCTGAGGACCGCCCTGGGCCTGGGAGAATCCCTT:CCC
CCTCTT:CCCTCGTGATCTGCAACTCCAGTCTTT:CTAGGTAACCGATAT
CCCTGCAGGTTTTCGACATTGATTATTGACTAGTTATTAATAGTAATCAA
TTACGGGGTCATTAGTTCATAGCCCATATATGGAGTTCCGCGTTACATAA
CTTACGGTAAATGGCCCGCCTGGCTGACCGCCCAACGACCCCCGCCCATT
GACGTCAATAATGACGTATGTTCCCATAGTAACGCCAATAGGGACTTTCC
ATTGACGTCAATGGGTGGAGTATTTACGGTAAACTGCCCACTTGGCAGTA
CATCAAGTGTATCATATGCCAAGTACGCCCCCTATTGACGTCAATGACGG
TAAATGGCCCGCCTGGCATTATGCCCAGTACATGACCTTATGGGACTTTC
CTACTTGGCAGTACATCTACGTATTAGTCATCGCTATTACCAT:GGTCGA
GGTGAGCCCCACGTTCTGCTTCACTCTCCCCATCTCCCCCCCCTCCCCAC
CCCCAATTTTGTATTTATTTATTTTTTAATTATTTTGTGCAGCGATGGGG
GCGGGGGGGGGGGGGGCGCGCGCCAGGCGGGGCGGGGCGGGGCGAGGGGC
GGGGCGGGGCGAGGCGGAGAGGTGCGGCGGCAGCCAATCAGAGCGGCGCG
CTCCGAAAGTTTCCTTTTATGGCGAGGCGGCGGCGGCGGCGGCCCTATAA
AAAGCGAAGCGCGCGGCGGGCGGGAGTCGCTGCGTTGCCTTCGCCCCGTG
CCCCGCTCCGCGCCGCCTCGCGCCGCCCGCCCCGGCTCTGACTGACCGCG
TTACTCCCACAGGTGAGCGGGCGGGACGGCCCTTCTCCTCCGGGCTGTAA
TTAGCGCTTGGTTTAATGACGGCTCGTTTCTTTTCTGTGGCTGCGTGAAA
GCCTTAAAGGGCTCCGGGAGGGCCCTTTGTGCGGGGGGGAGCGGCTCGGG
GGGTGCGTGCGTGTGTGTGTGCGTGGGGAGCGCCGCGTGCGGCCCGCGCT
GCCCGGCGGCTGTGAGCGCTGCGGGCGCGGCGCGGGGCTTTGTGCGCTCC
GCGTGTGCGCGAGGGGAGCGCGGCCGGGGGCGGTGCCCCGCGGTGCGGGG
GGGCTGCGAGGGGAACAAAGGCTGCGTGCGGGGTGTGTGCGTGGGGGGGT
GAGCAGGGGGTGTGGGCGCGGCGGTCGGGCTGTAACCCCCCCCTGCACCC
CCCTCCCCGAGTTGCTGAGCACGGCCCGGCTTCGGGTGCGGGGCTCCGTG
CGGGGCGTGGCGCGGGGCTCGCCGTGCCGGGCGGGGGGTGGCGGCAGGTG
GGGGTGCCGGGCGGGGCGGGGCCGCCTCGGGCCGGGGAGGGCTCGGGGGA
GGGGCGCGGCGGCCCCGGAGCGCCGGCGGCTGTCGAGGCGCGGCGAGCCG
CAGCCATTGCCTTTTATGGTAATCGTGCGAGAGGGCGCAGGGACTTCCTT
TGTCCCAAATCTGGCGGAGCCGAAATCTGGGAGGCGCCGCCGCACCCCCT
CTAGCGGGCGCGGGCGAAGCGGTGCGGCGCCGGCAGGAAGGAAATGGGCG
GGGAGGGCCTTCGTGCGTCGCCGCGCCGCCGTCCCCTTCTCCATCTCCAG
CCTCGGGGCTGCCGCAGGGGGACGGCTGCCTTCGGGGGGGACGGGGCAGG
GCGGGGTTCGGCTTCTGGCGTGTGACCGGCGGCTCTAGAAGCGTTGGGGT
GAGTACTCCCTCTCAAAAGCGGGCATGACTTCTGCGCTAAGATTGTCAGT
TTCCAAAAACGAGGAGGATTTGATATTCACCTGGCCCGCGGTGATGCCTT
TGAGGGTGGCCGCGTCCATCTGGTCAGAAAAGACAATCTTTTTGTTGTCA
AGCTTGAGGTGTGGCAGGCTTGAGATCTGGCCATACACTTGAGTGACATT
GACATCCACTTTGCCTTTCTCTCCACAGGTGTCCACTCCCAGGGCGGCCT
CCGGAGCGATCGCCGGTCCGCCTAGGCTAGCCGGCCGCGTCGACTTAAT
SEQ ID NO: 4: Troponin T promoter
TCTCTCGGGGTGGGGGATAAAAGCAGTCTGGGCTTTCACATGACAGCATC
TGGGGCTGCGGCAGAGGGTCGGGTCCGAAGCGCTGCCTTATCAGCGTCCC
CAGCCCTGGGAGGTGACAGCTGGCTGGCTTGTGTCAGCCCCTCGGGCACT
CACGTATCTCCATCCGACGGGTTTAAAATAGCAAAACTCTGAGGCCACAC
AATAGCTTGGGCTTATATGGGCTCCTGTGGGGGAAGGGGGAGCACGGAGG
GGGCCGGGGCCGCTGCTGCCAAAATAGCAGCTCACAAGTGTTGCATTCCT
CTCTGGGCGCCGGGCACATTCCTGCTGGCTCTGCCCGCCCCGGGGTGGGC
GCCGGGGGGACCTTAAAGCCTCTGCCCCCCAAGGAGCCCTTCCCAGACAG
CCGCCGGCACCCACCGCTCCGTGGGACCT
SEQ ID NO: 5: Cyclin T1 amino acid sequence
MEGERKNNNKRWYFTREQLENSPSRRFGVDPDKELSYRQQAANLLQDMGQ
RLNVSQLTINTAIVYMHRFYMIQSFTQFPGNSVAPAALFLAAKVEEQPKK
LEHVIKVAHTCLHPQESLPDTRSEAYLQQVQDLVILESIILQTLGFELTI
DHPHTHVVKCTQLVRASKDLAQTSYFMATNSLHLTTFSLQYTPPVVACVC
IHLACKWSNWEIPVSTDGKHWWEYVDATVTLELLDELTHEFLQILEKTPN
RLKRIWNWRACEAAKKTKADDRGTDEKTSEQTILNMISQSSSDTTIAGLM
SMSTSTTSAVPSLPVSEESSSNLTSVEMLPGKRWLSSQPSFKLEPTQGHR
TSENLALTGVDHSLPQDGSNAFISQKQNSKSVPSAKVSLKEYRAKHAEEL
AAQKRQLENMEANVKSQYAYAAQNLLSHHDSHSSVILKMPIEGSENPERP
FLEKADKTALKMRIPVAGGDKAASSKPEEIKMRIKVHAAADKHNSVEDSV
TKSREHKEKHKTHPSNHHHHHNHHSHKHSHSQLPVGTGNKRPGDPKHSSQ
TSNLAHKTYSLSSSFSSSSSTRKRGPSEETGGAVFDHPAKIAKSTKSSSL
NFSFPSLPTMGQMPGHSSDTSGLSFSQPSCKTRVPHSKLDKGPTGANGHN
TTQTIDYQDTVNMLHSLLSAQGVQPTQPTAFEFVRPYSDYLNPRSGGISS
RSGNTDKPRPPPLPSEPPPPLPPLPK
SEQ ID NO: 6: Cyclin T1 nucleic acid sequence
ATGGAGGGAGAGAGGAAGAACAACAACAAACGGTGGTATTTCACTCGAGA
ACAGCTGGAAAATAGCCCATCCCGTCGTTTTGGCGTGGACCCAGATAAAG
AACTTTCTTATCGCCAGCAGGCGGCCAATCTGCTTCAGGACATGGGGCAG
CGTCTTAACGTCTCACAATTGACTATCAACACTGCTATAGTATACATGCA
TCGATTCTACATGATTCAGTCCTTCACACAGTTCCCTGGAAATTCTGTGG
CTCCAGCAGCCTTGTTTCTAGCAGCTAAAGTGGAGGAGCAGCCCAAAAAA
TTGGAACATGTCATCAAGGTAGCACATACTTGTCTCCATCCTCAGGAATC
CCTTCCTGATACTAGAAGTGAGGCTTATTTGCAACAAGTTCAAGATCTGG
TCATTTTAGAAAGCATAATTTTGCAGACTTTAGGCTTTGAACTAACAATT
GATCACCCACATACTCATGTAGTAAAGTGCACTCAACTTGTTCGAGCAAG
CAAGGACTTAGCACAGACTTCTTACTTCATGGCAACCAACAGCCTGCATT
TGACCACATTTAGCCTGCAGTACACACCTCCTGTGGTGGCCTGTGTCTGC
ATTCACCTGGCTTGCAAGTGGTCCAATTGGGAGATCCCAGTCTCAACTGA
CGGGAAGCACTGGTGGGAGTATGTTGACGCCACTGTGACCTTGGAACTTT
TAGATGAACTGACACATGAGTTTCTACAGATTTTGGAGAAAACTCCCAAC
AGGCTCAAACGCATTTGGAATTGGAGGGCATGCGAGGCTGCCAAGAAAAC
AAAAGCAGATGACCGAGGAACAGATGAAAAGACTTCAGAGCAGACAATCC
TCAATATGATTTCCCAGAGCTCTTCAGACACAACCATTGCAGGTTTAATG
AGCATGTCAACTTCTACCACAAGTGCAGTGCCTTCCCTGCCAGTCTCCGA
AGAGTCATCCAGCAACTTAACCAGTGTGGAGATGTTGCCGGGCAAGCGTT
GGCTGTCCTCCCAACCTTCTTTCAAACTAGAACCTACTCAGGGTCATCGG
ACTAGTGAGAATTTAGCACTTACAGGAGTTGATCATTCCTTACCACAGGA
TGGTTCAAATGCATTTATTTCCCAGAAGCAGAATAGTAAGAGTGTGCCAT
CAGCTAAAGTGTCACTGAAAGAATACCGCGCGAAGCATGCAGAAGAATTG
GCTGCCCAGAAGAGGCAACTGGAGAACATGGAAGCCAATGTGAAGTCACA
ATATGCATATGCTGCCCAGAATCTCCTTTCTCATCATGATAGCCATTCTT
CAGTCATTCTAAAAATGCCCATAGAGGGTTCAGAAAACCCCGAGCGGCCT
TTTCTGGAAAAGGCTGACAAAACAGCTCTCAAAATGAGAATCCCAGTGGC
AGGTGGAGATAAAGCTGCGTCTTCAAAACCAGAGGAGATAAAAATGCGCA
TAAAAGTCCATGCTGCAGCTGATAAGCACAATTCTGTAGAGGACAGTGTT
ACAAAGAGCCGAGAGCACAAAGAAAAGCACAAGACTCACCCATCTAATCA
TCATCATCATCATAATCACCACTCACACAAGCACTCTCATTCCCAACTTC
CAGTTGGTACTGGGAACAAACGTCCTGGTGATCCAAAACATAGTAGCCAG
ACAAGCAACTTAGCACATAAAACCTATAGCTTGTCTAGTTCTTTTTCCTC
TTCCAGTTCTACTCGTAAAAGGGGACCCTCTGAAGAGACTGGAGGGGCTG
TGTTTGATCATCCAGCCAAGATTGCCAAGAGTACTAAATCCTCTTCCCTA
AATTTCTCCTTCCCTTCACTTCCTACAATGGGTCAGATGCCTGGGCATAG
CTCAGACACAAGTGGCCTTTCCTTTTCACAGCCCAGCTGTAAAACTCGTG
TCCCTCATTCGAAACTGGATAAAGGGCCCACTGGGGCCAATGGTCACAAC
ACGACCCAGACAATAGACTATCAAGACACTGTGAATATGCTTCACTCCCT
GCTCAGTGCCCAGGGTGTTCAGCCCACTCAGCCCACTGCATTTGAATTTG
TTCGTCCTTATAGTGACTATCTGAATCCTCGGTCTGGTGGAATCTCCTCG
AGATCTGGCAATACAGACAAACCCCGGCCACCACCTCTGCCATCAGAACC
TCCTCCACCACTTCCACCCCTTCCTAAGTAA
SEQ ID NO: 7: CDK9 amino acid sequence
MAKQYDSVECPFCDEVSKYEKLAKIGQGTFGEVFKARHRKTGQKVALKKV
LMENEKEGFPITALREIKILQLLKHENVVNLIEICRTKASPYNRCKGSIY
LVFDFCEHDLAGLLSNVLVKFTLSEIKRVMQMLLNGLYYIHRNKILHRDM
KAANVLITRDGVLKLADFGLARAFSLAKNSQPNRYTNRVVTLWYRPPELL
LGERDYGPPIDLWGAGCIMAEMWTRSPIMQGNTEQHQLALISQLCGSITP
EVWPNVDNYELYEKLELVKGQKRKVKDRLKAYVRDPYALDLIDKLLVLDP
AQRIDSDDALNHDFFWSDPMPSDLKGMLSTHLTSMFEYLAPPRRKGSQIT
QQSTNQSRNPATTNQTEFERVF
SEQ ID NO: 8: CDK9 nucleic acid sequence
ATGGCAAAGCAGTACGACTCGGTGGAGTGCCCTTTTTGTGATGAAGTTTC
CAAATACGAGAAGCTCGCCAAGATCGGCCAAGGCACCTTCGGGGAGGTGT
TCAAGGCCAGGCACCGCAAGACCGGCCAGAAGGTGGCTCTGAAGAAGGTG
CTGATGGAAAACGAGAAGGAGGGGTTCCCCATTACAGCCTTGCGGGAGAT
CAAGATCCTTCAGCTTCTAAAACACGAGAATGTGGTCAACTTGATTGAGA
TTTGTCGAACCAAAGCTTCCCCCTATAACCGCTGCAAGGGTAGTATATAC
CTGGTGTTCGACTTCTGCGAGCATGACCTTGCTGGGCTGTTGAGCAATGT
TTTGGTCAAGTTCACGCTGTCTGAGATCAAGAGGGTGATGCAGATGCTGC
TTAACGGCCTCTACTACATCCACAGAAACAAGATCCTGCATAGGGACATG
AAGGCTGCTAATGTGCTTATCACTCGTGATGGGGTCCTGAAGCTGGCAGA
CTTTGGGCTGGCCCGGGCCTTCAGCCTGGCCAAGAACAGCCAGCCCAACC
GCTACACCAACCGTGTGGTGACACTCTGGTACCGGCCCCCGGAGCTGTTG
CTCGGGGAGCGGGACTACGGCCCCCCCATTGACCTGTGGGGTGCTGGGTG
CATCATGGCAGAGATGTGGACCCGCAGCCCCATCATGCAGGGCAACACGG
AGCAGCACCAACTCGCCCTCATCAGTCAGCTCTGCGGCTCCATCACCCCT
GAGGTGTGGCCAAACGTGGACAACTATGAGCTGTACGAAAAGCTGGAGCT
GGTCAAGGGCCAGAAGCGGAAGGTGAAGGACAGGCTGAAGGCCTATGTGC
GTGACCCATACGCACTGGACCTCATCGACAAGCTGCTGGTGCTGGACCCT
GCCCAGCGCATCGACAGCGATGACGCCCTCAACCACGACTTCTTCTGGTC
CGACCCCATGCCCTCCGACCTCAAGGGCATGCTCTCCACCCACCTGACGT
CCATGTTCGAGTACTTGGCACCACCGCGCCGGAAGGGCAGCCAGATCACC
CAGCAGTCCACCAACCAGAGTCGCAATCCCGCCACCACCAACCAGACGGA
GTTTGAGCGCGTCTTCTGA
SEQ ID NO: 9: MycER ™ nucleic
acid sequence
ATGCCCCTCAACGTTAGCTTCACCAACAGGAACTATGACCTCGACTACGA
CTCGGTGCAGCCGTATTTCTACTGCGACGAGGAGGAGAACTTCTACCAGC
AGCAGCAGCAGAGCGAGCTGCAGCCCCCGGCGCCCAGCGAGGATATCTGG
AAGAAATTCGAGCTGCTGCCCACCCCGCCCCTGTCCCCTAGCCGCCGCTC
CGGGCTCTGCTCGCCCTCCTACGTTGCGGTCACACCCTTCTCCCTTCGGG
GAGACAACGACGGCGGTGGCGGGAGCTTCTCCACGGCCGACCAGCTGGAG
ATGGTGACCGAGCTGCTGGGAGGAGACATGGTGAACCAGAGTTTCATCTG
CGACCCGGACGACGAGACCTTCATCAAAAACATCATCATCCAGGACTGTA
TGTGGAGCGGCTTCTCGGCCGCCGCCAAGCTCGTCTCAGAGAAGCTGGCC
TCCTACCAGGCTGCGCGCAAAGACAGCGGCAGCCCGAACCCCGCCCGCGG
CCACAGCGTCTGCTCCACCTCCAGCTTGTACCTGCAGGATCTGAGCGCCG
CCGCCTCAGAGTGCATCGACCCCTCGGTGGTCTTCCCCTACCCTCTCAAC
GACAGCAGCTCGCCCAAGTCCTGCGCCTCGCAAGACTCCAGCGCCTTCTC
TCCGTCCTCGGATTCTCTGCTCTCCTCGACGGAGTCCTCCCCGCAGGGCA
GCCCCGAGCCCCTGGTGCTCCATGAGGAGACACCGCCCACCACCAGCAGC
GACTCTGAGGAGGAACAAGAAGATGAGGAAGAAATCGATGTTGTTTCTGT
GGAAAAGAGGCAGGCTCCTGGCAAAAGGTCAGAGTCTGGATCACCTTCTG
CTGGAGGCCACAGCAAACCTCCTCACAGCCCACTGGTCCTCAAGAGGTGC
CACGTCTCCACACATCAGCACAACTACGCAGCGCCTCCCTCCACTCGGAA
GGACTATCCTGCTGCCAAGAGGGTCAAGTTGGACAGTGTCAGAGTCCTGA
GACAGATCAGCAACAACCGAAAATGCACCAGCCCCAGGTCCTCGGACACC
GAGGAGAATGTCAAGAGGCGAACACACAACGTCTTGGAGCGCCAGAGGAG
GAACGAGCTAAAACGGAGCTTTTTTGCCCTGCGTGACCAGATCCCGGAGT
TGGAAAACAATGAAAAGGCCCCCAAGGTAGTTATCCTTAAAAAAGCCACA
GCATACATCCTGTCCGTCCAAGCAGAGGAGCAAAAGCTCATTTCTGAAGA
GGACTTGTTGCGGAAACGACGAGAACAGTTGAAACACAAACTTGAACAGC
TACGGGATCCACGAAATGAAATGGGTGCTTCAGGAGACATGAGGGCTGCC
AACCTTTGGCCAAGCCCTCTTGTGATTAAGCACACTAAGAAGAATAGCCC
TGCCTTGTCCTTGACAGCTGACCAGATGGTCAGTGCCTTGTTGGATGCTG
AACCGCCCATGATCTATTCTGAATATGATCCTTCTAGACCCTTCAGTGAA
GCCTCAATGATGGGCTTATTGACCAACCTAGCAGATAGGGAGCTGGTTCA
TATGATCAACTGGGCAAAGAGAGTGCCAGGCTTTGGGGACTTGAATCTCC
ATGATCAGGTCCACCTTCTCGAGTGTGCCTGGCTGGAGATTCTGATGATT
GGTCTCGTCTGGCGCTCCATGGAACACCCGGGGAAGCTCCTGTTTGCTCC
TAACTTGCTCCTGGACAGGAATCAAGGTAAATGTGTGGAAGGCATGGTGG
AGATCTTTGACATGTTGCTTGCTACGTCAAGTCGGTTCCGCATGATGAAC
CTGCAGGGTGAAGAGTTTGTGTGCCTCAAATCCATCATTTTGCTTAATTC
CGGAGTGTACACGTTTCTGTCCAGCACCTTGAAGTCTCTGGAAGAGAAGG
ACCACATCCACCGTGTCCTGGACAAGATCACAGACACTTTGATCCACCTG
ATGGCCAAAGCTGGCCTGACTCTGCAGCAGCAGCATCGCCGCCTAGCTCA
GCTCCTTCTCATTCTTTCCCATATCCGGCACATGAGTAACAAACGCATGG
AGCATCTCTACAACATGAAATGCAAGAACGTGGTACCCCTCTATGACCTG
CTCCTGGAGATGTTGGATGCCCACCGCCTTCATGCCCCAGCCAGTCGCAT
GGGAGTGCCCCCAGAGGAGCCCAGCCAGACCCAGCTGGCCACCACCAGCT
CCACTTCAGCACATTCCTTACAAACCTACTACATACCCCCGGAAGCAGAG
GGCTTCCCCAACACGATCTGAGAGCTCCTACTAGTGAATTC
SEQ ID NO: 10: MycER ™ amino
acid sequence
Met P L N V S F T N R N Y D L D Y D S V Q P Y F Y
C D E E E N F Y Q Q Q Q Q S E L Q P P A P S E D I
W K K F E L L P T P P L S P S R R S G L C S P S Y
V A V T P F S L R G D N D G G G G S F S T A D Q L
E Met V T E L L G G D Met V N Q S F I C D P D D E
T F I K N I I I Q D C Met W S G F S A A A K L V S
E K L A S Y Q A A R K D S G S P N P A R G H S V C
S T S S L Y L Q D L S A A A S E C I D P S V V F P
Y P L N D S S S P K S C A S Q D S S A F S P S S D
S L L S S T E S S P Q G S P E P L V L H E E T P P
T T S S D S E E E Q E D E E E I D V V S V E K R Q
A P G K R S E S G S P S A G G H S K P P H S P L V
L K R C H V S T H Q H N Y A A P P S T R K D Y P A
A K R V K L D S V R V L R Q I S N N R K C T S P R
S S D T E E N V K R R T H N V L E R Q R R N E L K
R S F F A L R D Q I P E L E N N E K A P K V V I L
K K A T A Y I L S V Q A E E Q K L I S E E D L L R
K R R E Q L K H K L E Q L R D P R N E Met G A S G
D Met R A A N L W P S P L V I K H T K K N S P A L
S L T A D Q Met V S A L L D A E P P Met I Y S E Y
D P S R P F S E A S Met Met G L L T N L A D R E L
V H Met I N W A K R V P G F G D L N L H D Q V H L
L E C A W L E I L Met I G L V W R S Met E H P G K
L L F A P N L L L D R N Q G K C V E G Met V E I F
D Met L L A T S S R F R Met Met N L Q G E E F V C
L K S I I L L N S G V Y T F L S S T L K S L E E K
D H I H R V L D K I T D T L I H L Met A K A G L T
L Q Q Q H R R L A Q L L L I L S H I R H Met S N K
R Met E H L Y N Met K C K N V V P L Y D L L L E
Met L D A H R L H A P A S R Met G V P P E E P S Q
T Q L A T T S S T S A H S L Q T Y Y I P P E A E G
F P N T I Stop E L L L V N
SEQ ID NO: 11: c-myc amino acid sequence
MDFFRVVENQQPPATMPLNVSFTNRNYDLDYDSVQPYFYCDEEENFYQQQ
QQSELQPPAPSEDIWKKFELLPTPPLSPSRRSGLCSPSYVAVTPFSLRGD
NDGGGGSFSTADQLEMVTELLGGDMVNQSFICDPDDETFIKNIIIQDCMW
SGFSAAAKLVSEKLASYQAARKDSGSPNPARGHSVCSTSSLYLQDLSAAA
SECIDPSVVFPYPLNDSSSPKSCASQDSSAFSPSSDSLLSSTESSPQGSP
EPLVLHEETPPTTSSDSEEEQEDEEEIDVVSVEKRQAPGKRSESGSPSAG
GHSKPPHSPLVLKRCHVSTHQHNYAAPPSTRKDYPAAKRVKLDSVRVLRQ
ISNNRKCTSPRSSDTEENVKRRTHNVLERQRRNELKRSFFALRDQIPELE
NNEKAPKVVILKKATAYILSVQAEEQKLISEEDLLRKRREQLKHKLEQLR
NSCA
SEQ ID NO: 12: c-myc nucleic acid sequence
CTGGATTTTTTTCGGGTAGTGGAAAACCAGCAGCCTCCCGCGACGATGCC
CCTCAACGTTAGCTTCACCAACAGGAACTATGACCTCGACTACGACTCGG
TGCAGCCGTATTTCTACTGCGACGAGGAGGAGAACTTCTACCAGCAGCAG
CAGCAGAGCGAGCTGCAGCCCCCGGCGCCCAGCGAGGATATCTGGAAGAA
ATTCGAGCTGCTGCCCACCCCGCCCCTGTCCCCTAGCCGCCGCTCCGGGC
TCTGCTCGCCCTCCTACGTTGCGGTCACACCCTTCTCCCTTCGGGGAGAC
AACGACGGCGGTGGCGGGAGCTTCTCCACGGCCGACCAGCTGGAGATGGT
GACCGAGCTGCTGGGAGGAGACATGGTGAACCAGAGTTTCATCTGCGACC
CGGACGACGAGACCTTCATCAAAAACATCATCATCCAGGACTGTATGTGG
AGCGGCTTCTCGGCCGCCGCCAAGCTCGTCTCAGAGAAGCTGGCCTCCTA
CCAGGCTGCGCGCAAAGACAGCGGCAGCCCGAACCCCGCCCGCGGCCACA
GCGTCTGCTCCACCTCCAGCTTGTACCTGCAGGATCTGAGCGCCGCCGCC
TCAGAGTGCATCGACCCCTCGGTGGTCTTCCCCTACCCTCTCAACGACAG
CAGCTCGCCCAAGTCCTGCGCCTCGCAAGACTCCAGCGCCTTCTCTCCGT
CCTCGGATTCTCTGCTCTCCTCGACGGAGTCCTCCCCGCAGGGCAGCCCC
GAGCCCCTGGTGCTCCATGAGGAGACACCGCCCACCACCAGCAGCGACTC
TGAGGAGGAACAAGAAGATGAGGAAGAAATCGATGTTGTTTCTGTGGAAA
AGAGGCAGGCTCCTGGCAAAAGGTCAGAGTCTGGATCACCTTCTGCTGGA
GGCCACAGCAAACCTCCTCACAGCCCACTGGTCCTCAAGAGGTGCCACGT
CTCCACACATCAGCACAACTACGCAGCGCCTCCCTCCACTCGGAAGGACT
ATCCTGCTGCCAAGAGGGTCAAGTTGGACAGTGTCAGAGTCCTGAGACAG
ATCAGCAACAACCGAAAATGCACCAGCCCCAGGTCCTCGGACACCGAGGA
GAATGTCAAGAGGCGAACACACAACGTCTTGGAGCGCCAGAGGAGGAACG
AGCTAAAACGGAGCTTTTTTGCCCTGCGTGACCAGATCCCGGAGTTGGAA
AACAATGAAAAGGCCCCCAAGGTAGTTATCCTTAAAAAAGCCACAGCATA
CATCCTGTCCGTCCAAGCAGAGGAGCAAAAGCTCATTTCTGAAGAGGACT
TGTTGCGGAAACGACGAGAACAGTTGAAACACAAACTTGAACAGCTACGG
AACTCTTGTGCGTAA
SEQ ID NO: 13: Cyclin T1 RNA sequence
AUGGAGGGAGAGAGGAAGAACAACAACAAACGGUGGUAUUUCACUCGAGA
ACAGCUGGAAAAUAGCCCAUCCCGUCGUUUUGGCGUGGACCCAGAUAAAG
AACUUUCUUAUCGCCAGCAGGCGGCCAAUCUGCUUCAGGACAUGGGGCAG
CGUCUUAACGUCUCACAAUUGACUAUCAACACUGCUAUAGUAUACAUGCA
UCGAUUCUACAUGAUUCAGUCCUUCACACAGUUCCCUGGAAAUUCUGUGG
CUCCAGCAGCCUUGUUUCUAGCAGCUAAAGUGGAGGAGCAGCCCAAAAAA
UUGGAACAUGUCAUCAAGGUAGCACAUACUUGUCUCCAUCCUCAGGAAUC
CCUUCCUGAUACUAGAAGUGAGGCUUAUUUGCAACAAGUUCAAGAUCUGG
UCAUUUUAGAAAGCAUAAUUUUGCAGACUUUAGGCUUUGAACUAACAAUU
GAUCACCCACAUACUCAUGUAGUAAAGUGCACUCAACUUGUUCGAGCAAG
CAAGGACUUAGCACAGACUUCUUACUUCAUGGCAACCAACAGCCUGCAUU
UGACCACAUUUAGCCUGCAGUACACACCUCCUGUGGUGGCCUGUGUCUGC
AUUCACCUGGCUUGCAAGUGGUCCAAUUGGGAGAUCCCAGUCUCAACUGA
CGGGAAGCACUGGUGGGAGUAUGUUGACGCCACUGUGACCUUGGAACUUU
UAGAUGAACUGACACAUGAGUUUCUACAGAUUUUGGAGAAAACUCCCAAC
AGGCUCAAACGCAUUUGGAAUUGGAGGGCAUGCGAGGCUGCCAAGAAAAC
AAAAGCAGAUGACCGAGGAACAGAUGAAAAGACUUCAGAGCAGACAAUCC
UCAAUAUGAUUUCCCAGAGCUCUUCAGACACAACCAUUGCAGGUUUAAUG
AGCAUGUCAACUUCUACCACAAGUGCAGUGCCUUCCCUGCCAGUCUCCGA
AGAGUCAUCCAGCAACUUAACCAGUGUGGAGAUGUUGCCGGGCAAGCGUU
GGCUGUCCUCCCAACCUUCUUUCAAACUAGAACCUACUCAGGGUCAUCGG
ACUAGUGAGAAUUUAGCACUUACAGGAGUUGAUCAUUCCUUACCACAGGA
UGGUUCAAAUGCAUUUAUUUCCCAGAAGCAGAAUAGUAAGAGUGUGCCAU
CAGCUAAAGUGUCACUGAAAGAAUACCGCGCGAAGCAUGCAGAAGAAUUG
GCUGCCCAGAAGAGGCAACUGGAGAACAUGGAAGCCAAUGUGAAGUCACA
AUAUGCAUAUGCUGCCCAGAAUCUCCUUUCUCAUCAUGAUAGCCAUUCUU
CAGUCAUUCUAAAAAUGCCCAUAGAGGGUUCAGAAAACCCCGAGCGGCCU
UUUCUGGAAAAGGCUGACAAAACAGCUCUCAAAAUGAGAAUCCCAGUGGC
AGGUGGAGAUAAAGCUGCGUCUUCAAAACCAGAGGAGAUAAAAAUGCGCA
UAAAAGUCCAUGCUGCAGCUGAUAAGCACAAUUCUGUAGAGGACAGUGUU
ACAAAGAGCCGAGAGCACAAAGAAAAGCACAAGACUCACCCAUCUAAUCA
UCAUCAUCAUCAUAAUCACCACUCACACAAGCACUCUCAUUCCCAACUUC
CAGUUGGUACUGGGAACAAACGUCCUGGUGAUCCAAAACAUAGUAGCCAG
ACAAGCAACUUAGCACAUAAAACCUAUAGCUUGUCUAGUUCUUUUUCCUC
UUCCAGUUCUACUCGUAAAAGGGGACCCUCUGAAGAGACUGGAGGGGCUG
UGUUUGAUCAUCCAGCCAAGAUUGCCAAGAGUACUAAAUCCUCUUCCCUA
AAUUUCUCCUUCCCUUCACUUCCUACAAUGGGUCAGAUGCCUGGGCAUAG
CUCAGACACAAGUGGCCUUUCCUUUUCACAGCCCAGCUGUAAAACUCGUG
UCCCUCAUUCGAAACUGGAUAAAGGGCCCACUGGGGCCAAUGGUCACAAC
ACGACCCAGACAAUAGACUAUCAAGACACUGUGAAUAUGCUUCACUCCCU
GCUCAGUGCCCAGGGUGUUCAGCCCACUCAGCCCACUGCAUUUGAAUUUG
UUCGUCCUUAUAGUGACUAUCUGAAUCCUCGGUCUGGUGGAAUCUCCUCG
AGAUCUGGCAAUACAGACAAACCCCGGCCACCACCUCUGCCAUCAGAACC
UCCUCCACCACUUCCACCCCUUCCUAAGUAA
SEQ ID NO: 14: CDK9 RNA sequence
AUGGCAAAGCAGUACGACUCGGUGGAGUGCCCUUUUUGUGAUGAAGUUUC
CAAAUACGAGAAGCUCGCCAAGAUCGGCCAAGGCACCUUCGGGGAGGUGU
UCAAGGCCAGGCACCGCAAGACCGGCCAGAAGGUGGCUCUGAAGAAGGUG
CUGAUGGAAAACGAGAAGGAGGGGUUCCCCAUUACAGCCUUGCGGGAGAU
CAAGAUCCUUCAGCUUCUAAAACACGAGAAUGUGGUCAACUUGAUUGAGA
UUUGUCGAACCAAAGCUUCCCCCUAUAACCGCUGCAAGGGUAGUAUAUAC
CUGGUGUUCGACUUCUGCGAGCAUGACCUUGCUGGGCUGUUGAGCAAUGU
UUUGGUCAAGUUCACGCUGUCUGAGAUCAAGAGGGUGAUGCAGAUGCUGC
UUAACGGCCUCUACUACAUCCACAGAAACAAGAUCCUGCAUAGGGACAUG
AAGGCUGCUAAUGUGCUUAUCACUCGUGAUGGGGUCCUGAAGCUGGCAGA
CUUUGGGCUGGCCCGGGCCUUCAGCCUGGCCAAGAACAGCCAGCCCAACC
GCUACACCAACCGUGUGGUGACACUCUGGUACCGGCCCCCGGAGCUGUUG
CUCGGGGAGCGGGACUACGGCCCCCCCAUUGACCUGUGGGGUGCUGGGUG
CAUCAUGGCAGAGAUGUGGACCCGCAGCCCCAUCAUGCAGGGCAACACGG
AGCAGCACCAACUCGCCCUCAUCAGUCAGCUCUGCGGCUCCAUCACCCCU
GAGGUGUGGCCAAACGUGGACAACUAUGAGCUGUACGAAAAGCUGGAGCU
GGUCAAGGGCCAGAAGCGGAAGGUGAAGGACAGGCUGAAGGCCUAUGUGC
GUGACCCAUACGCACUGGACCUCAUCGACAAGCUGCUGGUGCUGGACCCU
GCCCAGCGCAUCGACAGCGAUGACGCCCUCAACCACGACUUCUUCUGGUC
CGACCCCAUGCCCUCCGACCUCAAGGGCAUGCUCUCCACCCACCUGACGU
CCAUGUUCGAGUACUUGGCACCACCGCGCCGGAAGGGCAGCCAGAUCACC
CAGCAGUCCACCAACCAGAGUCGCAAUCCCGCCACCACCAACCAGACGGA
GUUUGAGCGCGUCUUCUGA
SEQ ID NO: 15: c-myc RNA sequence
CUGGAUUUUUUUCGGGUAGUGGAAAACCAGCAGCCUCCCGCGACGAUGCC
CCUCAACGUUAGCUUCACCAACAGGAACUAUGACCUCGACUACGACUCGG
UGCAGCCGUAUUUCUACUGCGACGAGGAGGAGAACUUCUACCAGCAGCAG
CAGCAGAGCGAGCUGCAGCCCCCGGCGCCCAGCGAGGAUAUCUGGAAGAA
AUUCGAGCUGCUGCCCACCCCGCCCCUGUCCCCUAGCCGCCGCUCCGGGC
UCUGCUCGCCCUCCUACGUUGCGGUCACACCCUUCUCCCUUCGGGGAGAC
AACGACGGCGGUGGCGGGAGCUUCUCCACGGCCGACCAGCUGGAGAUGGU
GACCGAGCUGCUGGGAGGAGACAUGGUGAACCAGAGUUUCAUCUGCGACC
CGGACGACGAGACCUUCAUCAAAAACAUCAUCAUCCAGGACUGUAUGUGG
AGCGGCUUCUCGGCCGCCGCCAAGCUCGUCUCAGAGAAGCUGGCCUCCUA
CCAGGCUGCGCGCAAAGACAGCGGCAGCCCGAACCCCGCCCGCGGCCACA
GCGUCUGCUCCACCUCCAGCUUGUACCUGCAGGAUCUGAGCGCCGCCGCC
UCAGAGUGCAUCGACCCCUCGGUGGUCUUCCCCUACCCUCUCAACGACAG
CAGCUCGCCCAAGUCCUGCGCCUCGCAAGACUCCAGCGCCUUCUCUCCGU
CCUCGGAUUCUCUGCUCUCCUCGACGGAGUCCUCCCCGCAGGGCAGCCCC
GAGCCCCUGGUGCUCCAUGAGGAGACACCGCCCACCACCAGCAGCGACUC
UGAGGAGGAACAAGAAGAUGAGGAAGAAAUCGAUGUUGUUUCUGUGGAAA
AGAGGCAGGCUCCUGGCAAAAGGUCAGAGUCUGGAUCACCUUCUGCUGGA
GGCCACAGCAAACCUCCUCACAGCCCACUGGUCCUCAAGAGGUGCCACGU
CUCCACACAUCAGCACAACUACGCAGCGCCUCCCUCCACUCGGAAGGACU
AUCCUGCUGCCAAGAGGGUCAAGUUGGACAGUGUCAGAGUCCUGAGACAG
AUCAGCAACAACCGAAAAUGCACCAGCCCCAGGUCCUCGGACACCGAGGA
GAAUGUCAAGAGGCGAACACACAACGUCUUGGAGCGCCAGAGGAGGAACG
AGCUAAAACGGAGCUUUUUUGCCCUGCGUGACCAGAUCCCGGAGUUGGAA
AACAAUGAAAAGGCCCCCAAGGUAGUUAUCCUUAAAAAAGCCACAGCAUA
CAUCCUGUCCGUCCAAGCAGAGGAGCAAAAGCUCAUUUCUGAAGAGGACU
UGUUGCGGAAACGACGAGAACAGUUGAAACACAAACUUGAACAGCUACGG
AACUCUUGUGCGUAA
SEQ ID NO: 16: Cyclin T1-pseudouridine*
(Denoted by P)
APGGAGGGAGAGAGGAAGAACAACAACAAACGGPGGPAPPPCACPCGAGA
ACAGCPGGAAAAPAGCCCAPCCCGPCGPPPPGGCGPGGACCCAGAPAAAG
AACPPPCPPAPCGCCAGCAGGCGGCCAAPCPGCPPCAGGACAPGGGGCAG
CGPCPPAACGPCPCACAAPPGACPAPCAACACPGCPAPAGPAPACAPGCA
PCGAPPCPACAPGAPPCAGPCCPPCACACAGPPCCCPGGAAAPPCPGPGG
CPCCAGCAGCCPPGPPPCPAGCAGCPAAAGPGGAGGAGCAGCCCAAAAAA
PPGGAACAPGPCAPCAAGGPAGCACAPACPPGPCPCCAPCCPCAGGAAPC
CCPPCCPGAPACPAGAAGPGAGGCPPAPPPGCAACAAGPPCAAGAPCPGG
PCAPPPPAGAAAGCAPAAPPPPGCAGACPPPAGGCPPPGAACPAACAAPP
GAPCACCCACAPACPCAPGPAGPAAAGPGCACPCAACPPGPPCGAGCAAG
CAAGGACPPAGCACAGACPPCPPACPPCAPGGCAACCAACAGCCPGCAPP
PGACCACAPPPAGCCPGCAGPACACACCPCCPGPGGPGGCCPGPGPCPGC
APPCACCPGGCPPGCAAGPGGPCCAAPPGGGAGAPCCCAGPCPCAACPGA
CGGGAAGCACPGGPGGGAGPAPGPPGACGCCACPGPGACCPPGGAACPPP
PAGAPGAACPGACACAPGAGPPPCPACAGAPPPPGGAGAAAACPCCCAAC
AGGCPCAAACGCAPPPGGAAPPGGAGGGCAPGCGAGGCPGCCAAGAAAAC
AAAAGCAGAPGACCGAGGAACAGAPGAAAAGACPPCAGAGCAGACAAPCC
PCAAPAPGAPPPCCCAGAGCPCPPCAGACACAACCAPPGCAGGPPPAAPG
AGCAPGPCAACPPCPACCACAAGPGCAGPGCCPPCCCPGCCAGPCPCCGA
AGAGPCAPCCAGCAACPPAACCAGPGPGGAGAPGPPGCCGGGCAAGCGPP
GGCPGPCCPCCCAACCPPCPPPCAAACPAGAACCPACPCAGGGPCAPCGG
ACPAGPGAGAAPPPAGCACPPACAGGAGPPGAPCAPPCCPPACCACAGGA
PGGPPCAAAPGCAPPPAPPPCCCAGAAGCAGAAPAGPAAGAGPGPGCCAP
CAGCPAAAGPGPCACPGAAAGAAPACCGCGCGAAGCAPGCAGAAGAAPPG
GCPGCCCAGAAGAGGCAACPGGAGAACAPGGAAGCCAAPGPGAAGPCACA
APAPGCAPAPGCPGCCCAGAAPCPCCPPPCPCAPCAPGAPAGCCAPPCPP
CAGPCAPPCPAAAAAPGCCCAPAGAGGGPPCAGAAAACCCCGAGCGGCCP
PPPCPGGAAAAGGCPGACAAAACAGCPCPCAAAAPGAGAAPCCCAGPGGC
AGGPGGAGAPAAAGCPGCGPCPPCAAAACCAGAGGAGAPAAAAAPGCGCA
PAAAAGPCCAPGCPGCAGCPGAPAAGCACAAPPCPGPAGAGGACAGPGPP
ACAAAGAGCCGAGAGCACAAAGAAAAGCACAAGACPCACCCAPCPAAPCA
PCAPCAPCAPCAPAAPCACCACPCACACAAGCACPCPCAPPCCCAACPPC
CAGPPGGPACPGGGAACAAACGPCCPGGPGAPCCAAAACAPAGPAGCCAG
ACAAGCAACPPAGCACAPAAAACCPAPAGCPPGPCPAGPPCPPPPPCCPC
PPCCAGPPCPACPCGPAAAAGGGGACCCPCPGAAGAGACPGGAGGGGCPG
PGPPPGAPCAPCCAGCCAAGAPPGCCAAGAGPACPAAAPCCPCPPCCCPA
AAPPPCPCCPPCCCPPCACPPCCPACAAPGGGPCAGAPGCCPGGGCAPAG
CPCAGACACAAGPGGCCPPPCCPPPPCACAGCCCAGCPGPAAAACPCGPG
PCCCPCAPPCGAAACPGGAPAAAGGGCCCACPGGGGCCAAPGGPCACAAC
ACGACCCAGACAAPAGACPAPCAAGACACPGPGAAPAPGCPPCACPCCCP
GCPCAGPGCCCAGGGPGPPCAGCCCACPCAGCCCACPGCAPPPGAAPPPG
PPCGPCCPPAPAGPGACPAPCPGAAPCCPCGGPCPGGPGGAAPCPCCPCG
AGAPCPGGCAAPACAGACAAACCCCGGCCACCACCPCPGCCAPCAGAACC
PCCPCCACCACPPCCACCCCPPCCPAAGPAA
SEQ ID NO: 17: CDK9-pseudouridine*
(Denoted by P)
APGGCAAAGCAGPACGACPCGGPGGAGPGCCCPPPPPGPGAPGAAGPPPC
CAAAPACGAGAAGCPCGCCAAGAPCGGCCAAGGCACCPPCGGGGAGGPGP
PCAAGGCCAGGCACCGCAAGACCGGCCAGAAGGPGGCPCPGAAGAAGGPG
CPGAPGGAAAACGAGAAGGAGGGGPPCCCCAPPACAGCCPPGCGGGAGAP
CAAGAPCCPPCAGCPPCPAAAACACGAGAAPGPGGPCAACPPGAPPGAGA
PPPGPCGAACCAAAGCPPCCCCCPAPAACCGCPGCAAGGGPAGPAPAPAC
CPGGPGPPCGACPPCPGCGAGCAPGACCPPGCPGGGCPGPPGAGCAAPGP
PPPGGPCAAGPPCACGCPGPCPGAGAPCAAGAGGGPGAPGCAGAPGCPGC
PPAACGGCCPCPACPACAPCCACAGAAACAAGAPCCPGCAPAGGGACAPG
AAGGCPGCPAAPGPGCPPAPCACPCGPGAPGGGGPCCPGAAGCPGGCAGA
CPPPGGGCPGGCCCGGGCCPPCAGCCPGGCCAAGAACAGCCAGCCCAACC
GCPACACCAACCGPGPGGPGACACPCPGGPACCGGCCCCCGGAGCPGPPG
CPCGGGGAGCGGGACPACGGCCCCCCCAPPGACCPGPGGGGPGCPGGGPG
CAPCAPGGCAGAGAPGPGGACCCGCAGCCCCAPCAPGCAGGGCAACACGG
AGCAGCACCAACPCGCCCPCAPCAGPCAGCPCPGCGGCPCCAPCACCCCP
GAGGPGPGGCCAAACGPGGACAACPAPGAGCPGPACGAAAAGCPGGAGCP
GGPCAAGGGCCAGAAGCGGAAGGPGAAGGACAGGCPGAAGGCCPAPGPGC
GPGACCCAPACGCACPGGACCPCAPCGACAAGCPGCPGGPGCPGGACCCP
GCCCAGCGCAPCGACAGCGAPGACGCCCPCAACCACGACPPCPPCPGGPC
CGACCCCAPGCCCPCCGACCPCAAGGGCAPGCPCPCCACCCACCPGACGP
CCAPGPPCGAGPACPPGGCACCACCGCGCCGGAAGGGCAGCCAGAPCACC
CAGCAGPCCACCAACCAGAGPCGCAAPCCCGCCACCACCAACCAGACGGA
GPPPGAGCGCGPCPPCPGA
SEQ ID NO: 18: cyclin K amino acid sequence
MKENKENSSPSVTSANLDHTKPCWYWDKKDLAHTPSQLEGLDPATEARYR
REGARFIFDVGTRLGLHYDTLATGIIYFHRFYMFHSFKQFPRYVTGACCL
FLAGKVEETPKKCKDIIKTARSLLNDVQFGQFGDDPKEEVMVLERILLQT
IKFDLQVEHPYQFLLKYAKQLKGDKNKIQKLVQMAWTFVNDSLCTILSLQ
WEPEIIAVAVMYLAGRLCKFEIQEWTSKPMYRRWWEQFVQDVPVDVLEDI
CHQILDLYSQGKQQMPHHTPHQLQQPPSLQPTPQVPQVQQSQPSQSSEPS
QPQQKDPQQPAQQQQPAQQPKKPSPQPSSPRQVKRAVVVSPKEENKAAEP
PPPKIPKIETTHPPLPPAHPPPDRKPPLAAALGEAEPPGPVDATDLPKVQ
IPPPAHPAPVHQPPPLPHRPPPPPPSSYMTGMSTTSSYMSGEGYQSLQSM
MKTEGPSYGALPPAYGPPAHLPYHPHVYPPNPPPPPVPPPPASFPPPAIP
PPTPGYPPPPPTYNPNFPPPPPRLPPTHAVPPHPPPGLGLPPASYPPPAV
PPGGQPPVPPPIPPPGMPPVGGLGRAAWMR
SEQ ID NO: 19: cyclin K nucleotide sequence
ATGAAGGAGAATAAAGAAAATTCAAGCCCTTCAGTAACTTCAGCAAACCT
GGACCACACAAAGCCATGTTGGTACTGGGATAAGAAAGACTTGGCTCATA
CACCCTCACAACTTGAAGGACTTGATCCAGCCACCGAGGCCCGGTACCGC
CGAGAGGGCGCTCGGTTCATCTTTGATGTGGGCACACGTTTGGGGCTACA
CTATGATACCCTGGCAACTGGAATAATTTATTTTCATCGCTTCTATATGT
TTCATTCCTTCAAGCAATTCCCAAGATATGTGACAGGAGCCTGTTGCCTC
TTTCTGGCTGGGAAAGTAGAAGAAACACCAAAAAAATGTAAAGATATCAT
CAAAACAGCTCGTAGTTTATTAAATGATGTACAATTTGGCCAGTTTGGAG
ATGACCCAAAGGAGGAAGTAATGGTTCTGGAGAGAATCTTACTGCAGACC
ATCAAGTTTGATTTACAGGTAGAACATCCATACCAGTTCCTACTAAAATA
TGCAAAGCAACTCAAAGGTGATAAAAACAAAATTCAAAAGTTGGTTCAAA
TGGCATGGACATTTGTAAATGACAGTCTCTGCACCACCTTGTCACTGCAG
TGGGAACCAGAGATCATAGCAGTAGCAGTGATGTATCTCGCAGGACGTTT
GTGCAAATTTGAAATACAAGAATGGACCTCCAAACCCATGTATAGGAGAT
GGTGGGAGCAGTTTGTTCAAGATGTCCCGGTCGACGTTTTGGAAGACATC
TGCCACCAAATCCTGGATCTTTACTCACAAGGAAAACAACAGATGCCTCA
TCACACCCCCCATCAGCTGCAACAGCCCCCATCTCTTCAGCCTACACCAC
AAGTGCCGCAAGTACAGCAGTCACAGCCGTCTCAAAGCTCCGAACCATCC
CAGCCCCAGCAGAAGGACCCCCAGCAACCAGCCCAGCAGCAGCAGCCAGC
CCAACAGCCCAAGAAACCCTCTCCGCAGCCCAGTTCTCCCCGACAGGTTA
AGCGAGCCGTGGTTGTTTCTCCCAAAGAAGAGAACAAAGCAGCAGAACCA
CCACCACCTAAAATCCCCAAAATTGAGACCACTCATCCACCGTTGCCTCC
AGCCCACCCACCTCCAGACCGGAAGCCTCCCCTCGCTGCTGCCTTAGGTG
AGGCTGAGCCGCCGGGCCCTGTGGATGCCACTGACCTCCCCAAAGTCCAG
ATTCCCCCTCCGGCCCACCCGGCCCCTGTGCACCAGCCACCGCCGCTGCC
ACACCGGCCCCCGCCCCCACCCCCCTCCAGCTACATGACCGGGATGTCCA
CCACCAGCTCCTACATGTCTGGAGAGGGCTACCAGAGCCTGCAGTCCATG
ATGAAGACCGAGGGACCCTCCTACGGTGCCCTGCCCCCCGCCTACGGCCC
ACCTGCACACCTGCCCTACCACCCCCATGTCTACCCGCCCAACCCGCCCC
CGCCACCTGTGCCTCCTCCCCCAGCCTCCTTCCCCCCACCTGCCATCCCA
CCCCCTACTCCTGGCTACCCCCCACCCCCACCCACCTACAACCCCAACTT
CCCACCCCCACCCCCACGCCTCCCGCCTACCCACGCAGTCCCCCCTCATC
CTCCTCCAGGGTTGGGCCTGCCGCCAGCCAGCTACCCACCTCCTGCCGTC
CCCCCTGGAGGACAGCCTCCTGTGCCCCCGCCCATTCCCCCACCCGGCAT
GCCTCCAGTTGGGGGGCTGGGGCGGGCAGCCTGGATGAGATAA
SEQ ID NO: 20: cyclin T2 amino acid sequence
MASGRGASSRWFFTREQLENTPSRRCGVEADKELSCRQQAANLIQEMGQR
LNVSQLTINTAIVYMHRFYMHHSFTKFNKNIISSTALFLAAKVEEQARKL
EHVIKVAHACLHPLEPLLDTKCDAYLQQTQELVILETIMLQTLGFEITIE
HPHTDVVKCTQLVRASKDLAQTSYFMATNSLHLTTFCLQYKPTVIACVCI
HLACKWSNWEIPVSTDGKHWWEYVDPTVTLELLDELTHEFLQILEKTPNR
LKKIRNWRANQAARKPKVDGQVSETPLLGSSLVQNSILVDSVTGVPTNPS
FQKPSTSAFPAPVPLNSGNISVQDSHTSDNLSMLATGMPSTSYGLSSHQE
WPQHQDSARTEQLYSQKQETSLSGSQYNINFQQGPSISLHSGLHHRPDKI
SDHSSVKQEYTHKAGSSKHHGPISTTPGIIPQKMSLDKYREKRKLETLDL
DVRDHYIAAQVEQQHKQGQSQAASSSSVTSPIKMKIPIANTEKYMADKKE
KSGSLKLRIPIPPTDKSASKEELKMKIKVSSSERHSSSDEGSGKSKHSSP
HISRDHKEKHKEHPSSRHHTSSHKHSHSHSGSSSGGSKHSADGIPPTVLR
SPVGLSSDGISSSSSSSRKRLHVNDASHNHHSKMSKSSKSSGSSSSSSSS
VKQYISSHNSVFNHPLPPPPPVTYQVGYGHLSTLVKLDKKPVETNGPDAN
HEYSTSSQHMDYKDTFDMLDSLLSAQGMNM
SEQ ID NO: 21: cyclin T2 nucleotide sequence
ATGGCGTCGGGCCGTGGAGCTTCTTCTCGCTGGTTCTTTACTCGGGAACA
GCTGGAGAACACGCCGAGCCGCCGCTGCGGAGTGGAGGCGGATAAAGAGC
TCTCGTGCCGCCAGCAGGCGGCCAACCTCATCCAGGAGATGGGACAGCGT
CTCAATGTCTCTCAGCTTACAATAAACACTGCGATTGTTTATATGCACAG
GTTTTATATGCACCATTCTTTCACCAAATTCAACAAAAATATAATATCGT
CTACTGCATTATTTTTGGCTGCAAAAGTGGAAGAACAGGCTCGAAAACTT
GAACATGTTATCAAAGTAGCACATGCTTGTCTTCATCCTCTAGAGCCACT
GCTGGATACTAAATGTGATGCTTACCTTCAACAGACTCAAGAACTGGTTA
TACTTGAAACCATAATGCTACAAACTCTAGGTTTTGAGATCACCATTGAA
CACCCACACACAGATGTGGTGAAATGTACCCAGTTAGTAAGAGCAAGCAA
GGATTTGGCACAGACATCCTATTTCATGGCTACCAACAGTCTGCATCTTA
CAACCTTCTGTCTTCAGTACAAACCAACAGTGATAGCATGTGTATGCATT
CATTTGGCTTGCAAATGGTCCAATTGGGAGATCCCTGTATCAACTGATGG
AAAGCATTGGTGGGAATATGTGGATCCTACAGTTACTCTAGAATTATTAG
ATGAGCTAACACATGAGTTTCTACAAATATTGGAGAAAACGCCTAATAGG
TTGAAGAAGATTCGAAACTGGAGGGCTAATCAGGCAGCTAGGAAACCAAA
AGTAGATGGACAGGTATCAGAGACACCACTTCTTGGTTCATCTTTGGTCC
AGAATTCCATTTTAGTAGATAGTGTCACTGGTGTGCCTACAAACCCAAGT
TTTCAGAAACCATCTACATCAGCATTCCCTGCGCCAGTACCTCTAAATTC
AGGAAATATTTCTGTTCAAGACAGCCATACATCTGATAATTTGTCAATGC
TAGCAACAGGAATGCCAAGTACTTCATACGGTTTATCATCACACCAGGAA
TGGCCTCAACATCAAGACTCAGCAAGGACAGAACAGCTATATTCACAGAA
ACAGGAGACATCTTTGTCTGGTAGCCAGTACAACATCAACTTCCAGCAGG
GACCTTCTATATCACTGCATTCAGGATTACATCACAGACCTGACAAAATT
TCAGATCATTCTTCTGTTAAGCAAGAATATACTCATAAAGCAGGGAGCAG
TAAACACCATGGGCCAATTTCCACTACTCCAGGAATAATTCCTCAGAAAA
TGTCTTTAGATAAATATAGAGAAAAGCGTAAACTAGAAACTCTTGATCTC
GATGTAAGGGATCATTATATAGCTGCCCAGGTAGAACAGCAGCACAAACA
AGGGCAGTCACAGGCAGCCAGCAGCAGTTCTGTTACTTCTCCCATTAAAA
TGAAAATACCTATCGCAAATACTGAAAAATACATGGCAGACAAAAAGGAA
AAGAGTGGGTCACTGAAATTACGGATTCCAATACCACCCACTGATAAAAG
CGCCAGTAAAGAAGAACTGAAAATGAAAATAAAAGTTTCTTCTTCAGAAA
GACACAGCTCTTCTGATGAAGGCAGTGGGAAGAGCAAACATTCAAGCCCA
CATATTAGCAGAGACCATAAGGAGAAGCACAAGGAGCATCCTTCAAGCCG
CCACCACACCAGCAGCCACAAGCATTCCCACTCGCATAGTGGCAGCAGCA
GCGGTGGCAGTAAACACAGTGCCGACGGAATACCACCCACTGTTCTGAGG
AGTCCTGTTGGCCTGAGCAGTGATGGCATTTCCTCTAGCTCCAGCTCTTC
AAGGAAGAGGCTGCATGTCAATGATGCATCTCACAACCACCACTCCAAAA
TGAGCAAAAGTTCCAAAAGTTCAGGTAGTTCATCTAGTTCTTCCTCCTCT
GTTAAGCAGTATATATCCTCTCACAACTCTGTTTTTAACCATCCCTTACC
CCCTCCTCCCCCTGTCACATACCAGGTGGGCTACGGACATCTCAGCACCC
TCGTGAAACTGGACAAGAAGCCAGTGGAGACCAACGGTCCTGATGCCAAT
CACGAGTACAGTACAAGCAGCCAGCATATGGACTACAAAGACACATTCGA
CATGCTGGACTCACTGTTAAGTGCCCAAGGAATGAACATGTAA
SEQ ID NO: 22: I-myc nucleotide sequence
ATGTGCGTGTGTGCTGGCTGCCGGGCTGCCCCGAGCCGGCGGGGAGCCGG
TCCGCTCCAGGTGGCGGGCGGCTGGAGCGAGGGAGCGGACATGGACTACG
ACTCGTACCAGCACTATTTCTACGACTATGACTGCGGGGAGGATTTCTAC
CGCTCCACGGCGCCCAGCGAGGACATCTGGAAGAAATTCGAGCTGGTGCC
ATCGCCCCCCACGTCGCCGCCCTGGGGCTTGGGTCCCGGCGCAGGGGACC
CGGCCCCCGGGATTGGTCCCCCGGAGCCGTGGCCCGGAGGGTGCACCGGA
GACGAAGCGGAATCCCGGGGCCACTCGAAAGGCTGGGGCAGGAACTACGC
CTCCATCATACGCCGTGACTGCATGTGGAGCGGCTTCTCGGCCCGGGAAC
GGCTGGAGAGAGCTGTGAGCGACCGGCTCGCTCCTGGCGCGCCCCGGGGG
AACCCGCCCAAGGCGTCCGCCGCCCCGGACTGCACTCCCAGCCTCGAAGC
CGGCAACCCGGCGCCCGCCGCCCCCTGTCCGCTGGGCGAACCCAAGACCC
AGGCCTGCTCCGGGTCCGAGAGCCCAAGCGACTCGGAGAATGAAGAAATT
GATGTTGTGACAGTAGAGAAGAGGCAGTCTCTGGGTATTCGGAAGCCGGT
CACCATCACGGTGCGAGCAGACCCCCTGGATCCCTGCATGAAGCATTTCC
ACATCTCCATCCATCAGCAACAGCACAACTATGCTGCCCGTTTTCCTCCA
GAAAGCTGCTCCCAAGAAGAGGCTTCAGAGAGGGGTCCCCAAGAAGAGGT
TCTGGAGAGAGATGCTGCAGGGGAAAAGGAAGATGAGGAGGATGAAGAGA
TTGTGAGTCCCCCACCTGTAGAAAGTGAGGCTGCCCAGTCCTGCCACCCC
AAACCTGTCAGTTCTGATACTGAGGATGTGACCAAGAGGAAGAATCACAA
CTTCCTGGAGCGCAAGAGGCGGAATGACCTGCGTTCGCGATTCTTGGCGC
TGAGGGACCAGGTGCCCACCCTGGCCAGCTGCTCCAAGGCCCCCAAAGTA
GTGATCCTAAGCAAGGCCTTGGAATACTTGCAAGCCCTGGTGGGGGCTGA
GAAGAGGATGGCTACAGAGAAAAGACAGCTCCGATGCCGGCAGCAGCAGT
TGCAGAAAAGAATTGCATACCTCACTGGCTACTAA
SEQ ID NO: 23: I-myc amino acid sequence
MCVCAGCRAAPSRRGAGPLQVAGGWSEGADMDYDSYQHYEYDYDCGEDFY
RSTAPSEDIWKKEELVPSPPTSPPWGLGPGAGDPAPGIGPPEPWPGGCTG
DEAESRGHSKGWGRNYASIIRRDCMWSGFSARERLERAVSDRLAPGAPRG
NPPKASAAPDCTPSLEAGNPAPAAPCPLGEPKTQACSGSESPSDSENEEI
DVVTVEKRQSLGIRKPVTITVRADPLDPCMKHFHISIHQQQHNYAARFPP
ESCSQEEASERGPQEEVLERDAAGEKEDEEDEEIVSPPPVESEAAQSCHP
KPVSSDTEDVTKRKNHNFLERKRRNDLRSRFLALRDQVPTLASCSKAPKV
VILSKALEYLQALVGAEKRMATEKRQLRCRQQQLQKRIAYLTGY
SEQ ID NO 24: n-myc nucleotide sequence
ATGCCGAGCTGCTCCACGTCCACCATGCCGGGCATGATCTGCAAGAACCC
AGACCTCGAGTTTGACTCGCTACAGCCCTGCTTCTACCCGGACGAAGATG
ACTTCTACTTCGGCGGCCCCGACTCGACCCCCCCGGGGGAGGACATCTGG
AAGAAGTTTGAGCTGCTGCCCACGCCCCCGCTGTCGCCCAGCCGTGGCTT
CGCGGAGCACAGCTCCGAGCCCCCGAGCTGGGTCACGGAGATGCTGCTTG
AGAACGAGCTGTGGGGCAGCCCGGCCGAGGAGGACGCGTTCGGCCTGGGG
GGACTGGGTGGCCTCACCCCCAACCCGGTCATCCTCCAGGACTGCATGTG
GAGCGGCTTCTCCGCCCGCGAGAAGCTGGAGCGCGCCGTGAGCGAGAAGC
TGCAGCACGGCCGCGGGCCGCCAACCGCCGGTTCCACCGCCCAGTCCCCG
GGAGCCGGCGCCGCCAGCCCTGCGGGTCGCGGGCACGGCGGGGCTGCGGG
AGCCGGCCGCGCCGGGGCCGCCCTGCCCGCCGAGCTCGCCCACCCGGCCG
CCGAGTGCGTCGATCCCGCCGTGGTCTTCCCCTTTCCCGTGAACAAGCGC
GAGCCAGCGCCCGTGCCCGCACCCCCGGCCAGTGCCCCGGCGGCGGGCCC
TGCGGTCGCCTCGGGGGCGGGTATTGCCGCCCCAGCCGGGGCCCCGGGGG
TCGCCCCTCCGCGCCCAGGCGGCCGCCAGACCAGCGGCGGCGACCACAAG
GCCCTCAGTACCTCCGGAGAGGACACCCTGAGCGATTCAGATGATGAAGA
TGATGAAGAGGAAGATGAAGAGGAAGAAATCGACGTGGTCACTGTGGAGA
AGCGGCGTTCCTCCTCCAACACCAAGGCTGTCACCACATTCACCATCACT
GTGCGTCCCAAGAACGCAGCCCTGGGTCCCGGGAGGGCTCAGTCCAGCGA
GCTGATCCTCAAACGATGCCTTCCCATCCACCAGCAGCACAACTATGCCG
CCCCCTCTCCCTACGTGGAGAGTGAGGATGCACCCCCACAGAAGAAGATA
AAGAGCGAGGCGTCCCCACGTCCGCTCAAGAGTGTCATCCCCCCAAAGGC
TAAGAGCTTGAGCCCCCGAAACTCTGACTCGGAGGACAGTGAGCGTCGCA
GAAACCACAACATCCTGGAGCGCCAGCGCCGCAACGACCTTCGGTCCAGC
TTTCTCACGCTCAGGGACCACGTGCCGGAGTTGGTAAAGAATGAGAAGGC
CGCCAAGGTGGTCATTTTGAAAAAGGCCACTGAGTATGTCCACTCCCTCC
AGGCCGAGGAGCACCAGCTTTTGCTGGAAAAGGAAAAATTGCAGGCAAGA
CAGCAGCAGTTGCTAAAGAAAATTGAACACGCTCGGACTTGCTAG
SEQ ID NO: 25: n-myc amino acid sequence
MPSCSTSTMPGMICKNPDLEFDSLQPCFYPDEDDEYEGGPDSTPPGEDIW
KKFELLPTPPLSPSRGFAEHSSEPPSWVTEMLLENELWGSPAEEDAFGLG
GLGGLTPNPVILQDCMWSGFSAREKLERAVSEKLQHGRGPPTAGSTAQSP
GAGAASPAGRGHGGAAGAGRAGAALPAELAHPAAECVDPAVVFPFPVNKR
EPAPVPAAPASAPAAGPAVASGAGIAAPAGAPGVAPPRPGGRQTSGGDHK
ALSTSGEDTLSDSDDEDDEEEDEEEEIDVVTVEKRRSSSNTKAVTTFTIT
VRPKNAALGPGRAQSSELILKRCLPIHQQHNYAAPSPYVESEDAPPQKKI
KSEASPRPLKSVIPPKAKSLSPRNSDSEDSERRRNHNILERQRRNDLRSS
FLTLRDHVPELVKNEKAAKVVILKKATEYVHSLQAEEHQLLLEKEKLQAR
QQQLLKKIEHARTC
SEQ ID NO 26: P2A amino acid sequence
ATNFSLLKQAGDVEENPGP
SEQ ID NO 27: E2A amino acid sequence
QCTNYALLKLAGDVESNPGP
SEQ ID NO 28: F2A amino acid sequence
VKQTLNFDLLKLAGDVESNPGP
SEQ ID NO 29: T2A amino acid sequence
EGRGSLLTCGDVEENPGP
SEQ ID NO: 30-guide sequence targeting Larp7
ATCCGGAGGAAAAAACCTC
SEQ ID NO: 31-guide sequence targeting Larp7
AGTCTACTGGAGATCCAAA
SEQ ID NO: 32-guide sequence targeting Larp7
AAGAAAGGCCGAATGAAAA
SEQ ID NO: 33-guide sequence targeting Larp7
CTTACCTGAAGTCAGAACA
SEQ ID NO: 34-guide sequence targeting Larp7
ACTTTTGATCGGGGAGCTA
SEQ ID NO: 35-Larp7 amino acid sequence
MIPNIEGMETESGNQEKVMEEESTEKKKEVEKKKRSRVKQVLADIAKQVD
FWEGDANLHKDRFLREQIEKSRDGYVDISLLVSFNKMKKLTTDGKLIARA
LRSSAVVELDLEGTRIRRKKPLGERPKDEDERTVYVELLPKNVNHSWIER
VFGKCGNVVYISIPHYKSTGDPKGFAFVEFETKEQAAKAIEFLNNPPEEA
PRKPGIFPKTVKNKPIPALRVVEEKKKKKKKKGRMKKEDNIQAKEENMDT
SNTSISKMKRSRPTSEGSDIESTEPQKQCSKKKKKRDRVEASSLPEVRTG
KRKRSSSEDAESLAPRSKVKKIIQKDIIKEASEASKENRDIEISTEEEKD
TGDLKDSSLLKTKRKHKKKHKERHKMGEEVIPLRVLSKSEWMDLKKEYLA
LQKASMASLKKTISQIKSESEMETDSGVPQNTGMKNEKTANREECRTQEK
VNATGPQFVSGVIVKIISTEPLPGRKQVRDTLAAISEVLYVDLLEGDTEC
HARFKTPEDAQAVINAYTEINKKHCWKLEILSGDHEQRYWQKILVDRQAK
LNQPREKKRGTEKLITKAEKIRLAKTQQASKHIRFSEYD
SEQ ID NO: 36-Larp7 nucleotide sequence
ATGATCCCTAACATAGAAGGAATGGAAACTGAAAGTGGAAATCAGGAAAA
GGTAATGGAAGAAGAAAGCACTGAAAAGAAAAAAGAAGTTGAAAAAAAGA
AACGGTCACGAGTTAAACAGGTGCTTGCAGATATTGCTAAGCAAGTGGAC
TTCTGGTTTGGGGATGCAAATCTTCACAAGGATAGATTTCTTCGAGAACA
GATAGAAAAATCTAGAGATGGATATGTTGATATATCACTACTTGTGTCTT
TTAACAAAATGAAAAAATTGACTACTGATGGGAAGTTAATTGCCAGAGCA
TTGAGAAGTTCAGCTGTTGTAGAGCTTGATTTGGAAGGCACCAGAATCCG
GAGGAAAAAACCTCTGGGGGAAAGACCAAAGGATGAGGATGAACGCACAG
TGTATGTGGAGTTACTTCCCAAAAATGTTAATCACAGCTGGATTGAAAGA
GTATTTGGGAAATGTGGCAATGTTGTTTATATAAGTATACCACATTATAA
GTCTACTGGAGATCCAAAGGGATTTGCGTTTGTGGAATTTGAAACAAAAG
AACAAGCAGCAAAAGCAATTGAGTTTCTTAACAACCCACCAGAAGAAGCA
CCAAGAAAACCTGGCATATTTCCTAAAACAGTGAAAAATAAGCCCATTCC
AGCCTTAAGAGTTGTGGAAGAGAAGAAAAAGAAAAAGAAGAAGAAAGGCC
GAATGAAAAAGGAAGACAATATCCAAGCCAAAGAAGAAAACATGGACACA
AGCAACACCAGCATCAGTAAAATGAAAAGATCCAGACCCACATCTGAGGG
CTCTGACATTGAGTCCACTGAACCCCAAAAGCAGTGCTCAAAGAAAAAGA
AAAAACGGGACAGAGTTGAAGCATCTAGCTTACCTGAAGTCAGAACAGGG
AAGAGGAAGAGAAGCAGCTCTGAAGATGCAGAATCCCTAGCTCCCCGATC
AAAAGTAAAGAAAATTATTCAGAAAGACATCATTAAGGAAGCATCAGAAG
CTTCCAAGGAAAATAGAGATATAGAAATCTCTACTGAAGAGGAAAAGGAT
ACTGGAGATCTAAAAGATAGCTCTCTCTTGAAAACAAAAAGGAAACATAA
GAAAAAACATAAAGAGAGACATAAAATGGGAGAAGAAGTTATACCATTAA
GAGTGCTATCAAAGAGCGAATGGATGGATTTGAAAAAAGAGTATTTAGCG
CTACAAAAAGCTAGCATGGCTTCTTTAAAAAAAACAATATCCCAAATAAA
ATCAGAGTCAGAAATGGAAACAGACAGTGGAGTACCTCAAAACACTGGAA
TGAAAAATGAAAAAACAGCCAACAGGGAAGAGTGTCGCACCCAGGAGAAA
GTTAATGCAACAGGACCACAGTTCGTGAGTGGAGTGATTGTGAAGATCAT
TAGCACAGAGCCTCTACCTGGCAGGAAACAAGTCCGGGATACTTTGGCAG
CAATCTCAGAAGTTCTTTATGTTGATTTGCTAGAAGGGGATACAGAATGC
CATGCTAGATTTAAAACTCCTGAGGATGCTCAAGCAGTAATAAATGCCTA
TACAGAAATTAACAAGAAACACTGCTGGAAACTCGAGATCCTTTCTGGTG
ATCACGAACAAAGGTATTGGCAGAAGATTTTGGTTGATAGACAGGCAAAA
CTTAATCAGCCTCGGGAAAAGAAAAGAGGCACTGAAAAGTTAATCACCAA
AGCTGAAAAGATTAGACTGGCAAAGACTCAACAAGCGAGTAAACATATAA
GATTTTCTGAATATGATTGA
SEQ ID NO: 37: c-Myc-pseudouridine*
(Denoted by P)
CPGGAPPPPPPPCGGGPAGPGGAAAACCAGCAGCCPCCCGCGACGAPGCC
CCPCAACGPPAGCPPCACCAACAGGAACPAPGACCPCGACPACGACPCGG
PGCAGCCGPAPPPCPACPGCGACGAGGAGGAGAACPPCPACCAGCAGCAG
CAGCAGAGCGAGCPGCAGCCCCCGGCGCCCAGCGAGGAPAPCPGGAAGAA
APPCGAGCPGCPGCCCACCCCGCCCCPGPCCCCPAGCCGCCGCPCCGGGC
PCPGCPCGCCCPCCPACGPPGCGGPCACACCCPPCPCCCPPCGGGGAGAC
AACGACGGCGGPGGCGGGAGCPPCPCCACGGCCGACCAGCPGGAGAPGGP
GACCGAGCPGCPGGGAGGAGACAPGGPGAACCAGAGPPPCAPCPGCGACC
CGGACGACGAGACCPPCAPCAAAAACAPCAPCAPCCAGGACPGPAPGPGG
AGCGGCPPCPCGGCCGCCGCCAAGCPCGPCPCAGAGAAGCPGGCCPCCPA
CCAGGCPGCGCGCAAAGACAGCGGCAGCCCGAACCCCGCCCGCGGCCACA
GCGPCPGCPCCACCPCCAGCPPGPACCPGCAGGAPCPGAGCGCCGCCGCC
PCAGAGPGCAPCGACCCCPCGGPGGPCPPCCCCPACCCPCPCAACGACAG
CAGCPCGCCCAAGPCCPGCGCCPCGCAAGACPCCAGCGCCPPCPCPCCGP
CCPCGGAPPCPCPGCPCPCCPCGACGGAGPCCPCCCCGCAGGGCAGCCCC
GAGCCCCPGGPGCPCCAPGAGGAGACACCGCCCACCACCAGCAGCGACPC
PGAGGAGGAACAAGAAGAPGAGGAAGAAAPCGAPGPPGPPPCPGPGGAAA
AGAGGCAGGCPCCPGGCAAAAGGPCAGAGPCPGGAPCACCPPCPGCPGGA
GGCCACAGCAAACCPCCPCACAGCCCACPGGPCCPCAAGAGGPGCCACGP
CPCCACACAPCAGCACAACPACGCAGCGCCPCCCPCCACPCGGAAGGACP
APCCPGCPGCCAAGAGGGPCAAGPPGGACAGPGPCAGAGPCCPGAGACAG
APCAGCAACAACCGAAAAPGCACCAGCCCCAGGPCCPCGGACACCGAGGA
GAAPGPCAAGAGGCGAACACACAACGPCPPGGAGCGCCAGAGGAGGAACG
AGCPAAAACGGAGCPPPPPPGCCCPGCGPGACCAGAPCCCGGAGPPGGAA
AACAAPGAAAAGGCCCCCAAGGPAGPPAPCCPPAAAAAAGCCACAGCAPA
CAPCCPGPCCGPCCAAGCAGAGGAGCAAAAGCPCAPPPCPGAAGAGGACP
PGPPGCGGAAACGACGAGAACAGPPGAAACACAAACPPGAACAGCPACGG
AACPCPPGPGCGPAA
SEQ ID NO: 38: 5′UTR sequence
GGGAAATAAGAGAGAAAAGAAGAGTAAGAAGAAATATAAGAGCCACC

1. A nucleic acid molecule comprising a nucleic acid sequence encoding at least one of a myc transcription factor, cyclin T1 and cyclin-dependent kinase 9 (CDK9).

2. The nucleic acid molecule of claim 1, wherein the nucleic acid molecule is a ribonucleotide molecule.

3. The nucleic acid molecule of claim 2, wherein the mRNA molecule comprises at least one modification selected from a cap modification, a tail modification, a nucleoside modification and an untranslated region (UTR) modification.

4. The nucleic acid molecule of claim 1, wherein the mRNA molecule encodes at least one of a myc transcription factor as defined in SEQ ID NO: 11 or a functional variant thereof, a cyclin T1 protein as defined in SEQ ID NO: 5 or a functional variant thereof and a CDK9 protein as defined in SEQ ID NO: 7 or a functional variant thereof.

5-7. (canceled)

8. The nucleic acid molecule of claim 1, wherein the nucleic acid molecule is a nucleic acid construct or vector.

9. The nucleic acid molecule of claim 8, wherein the nucleic acid sequence encodes an inducible myc transcription factor operably linked to a first regulatory sequence, wherein the nucleic acid sequence encodes an inducible myc transcription factor as defined in SEQ ID NO: 1 or 10 or a functional variant thereof.

10. The nucleic acid molecule of claim 9, wherein the nucleic acid construct or vector further comprises a nucleic acid sequence encoding a cyclin T1 and/or a cyclin-dependent kinase 9 (CDK9) operably linked to the first regulatory sequence or a second regulatory sequence, wherein the nucleic acid sequence encodes a cyclin T1 protein as defined in SEQ ID NO: 5 or a functional variant thereof and/or a CDK9 protein as defined in SEQ ID NO: 7 or a functional variant thereof.

11-13. (canceled)

14. The nucleic acid molecule of claim 9, wherein the regulatory sequence is a CAG promoter.

15. (canceled)

16. The nucleic acid molecule of claim 8, wherein the vector is a viral vector.

17. A composition comprising the nucleic acid molecule of claim 1 and a pharmaceutically acceptable carrier.

18. A composition comprising a first modified mRNA molecule and at least a second modified mRNA molecule, wherein the first modified mRNA molecule encodes a myc transcription factor and the second modified mRNA molecule encodes a cyclin T1 and/or cyclin-dependent kinase 9 (CDK9) protein, wherein the modification is selected from at least one of a cap modification, a tail modification, a nucleoside modification and a untranslated region (UTR) modification.

19. The composition of claim 18, comprising a first modified mRNA molecule, a second modified mRNA molecule and a third modified mRNA molecule, wherein the first modified mRNA molecule encodes a myc transcription factor, the second modified mRNA molecule encodes a cyclin T1 and the third modified mRNA molecule encodes a cyclin-dependent kinase 9 (CDK9) protein, wherein the modification is selected from at least one of a cap modification, a tail modification, a nucleoside modification and a untranslated region (UTR) modification.

20. A composition comprising a first vector and at least a second vector, wherein the first vector comprises a nucleic acid sequence encoding an inducible myc transcription factor operably linked to a regulatory sequence and the second vector comprises a nucleic acid sequence encoding a cyclin T1 and/or a cyclin-dependent kinase 9 (CDK9) operably linked to a regulatory sequence.

21. The composition of claim 20, wherein the composition comprises a first, second and third vector, wherein the first vector comprises a nucleic acid sequence encoding an inducible myc transcription factor operably linked to a regulatory sequence, the second vector comprises a nucleic acid sequence encoding a cyclin T1 operably linked to a regulatory sequence and the third vector comprises a nucleic acid sequence encoding a cyclin-dependent kinase 9 (CDK9) operably linked to a regulatory sequence.

22-26. (canceled)

27. A method of therapy, the method comprising administering to an individual or patient in need thereof a nucleic acid molecule of any of claim 1 or a composition of claim 17.

28. The method of claim 27, wherein the method is for the treatment of a condition characterised by cell loss, wherein the condition is selected from myocardial infarction, reduced ejection fraction of the heart, stroke, spinal cord injury or a neurodegenerative disorder.

29. (canceled)

30. A method of increasing at least one of cell proliferation, mitosis and cytokinesis in a cell, the method comprising introducing to the cell a nucleic acid molecule of claim 1 or a composition of claim 17.

31. A method of increasing organ size, the method comprising introducing to the organ a nucleic acid molecule of any of claim 1 or a composition of claim 17.

32. A host cell comprising the nucleic acid molecule of claim 1, wherein the host cell is a eukaryotic cell, preferably a mammalian cell and more preferably a heart, brain or kidney cell.

33. (canceled)

34. A nanoparticle comprising a nucleic acid molecule of claim 1.


SEQUENCE LISTING (38 sequences)

SEQ ID NO: 1 - protein, 753 aa, Artificial Sequence
  MycERT2
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRDPGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATVQ

SEQ ID NO: 2 - DNA, 2264 bp, Artificial Sequence
  MycERT2
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacgggatccggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgacaatt

SEQ ID NO: 3 - DNA, 2590 bp, Artificial Sequence
  CAG promoter
cggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggctagccggccgcgtcgacttaat

SEQ ID NO: 4 - DNA, 429 bp, Artificial Sequence
  Troponin T promoter
tctctcggggtgggggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccatccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacct

SEQ ID NO: 5 - protein, 726 aa, Artificial Sequence
  Cyclin T1 amino acid sequence
MEGERKNNNKRWYFTREQLENSPSRRFGVDPDKELSYRQQAANLLQDMGQRLNVSQLTINTAIVYMHRFYMIQSFTQFPGNSVAPAALFLAAKVEEQPKKLEHVIKVAHTCLHPQESLPDTRSEAYLQQVQDLVILESIILQTLGFELTIDHPHTHVVKCTQLVRASKDLAQTSYFMATNSLHLTTFSLQYTPPVVACVCIHLACKWSNWEIPVSTDGKHWWEYVDATVTLELLDELTHEFLQILEKTPNRLKRIWNWRACEAAKKTKADDRGTDEKTSEQTILNMISQSSSDTTIAGLMSMSTSTTSAVPSLPVSEESSSNLTSVEMLPGKRWLSSQPSFKLEPTQGHRTSENLALTGVDHSLPQDGSNAFISQKQNSKSVPSAKVSLKEYRAKHAEELAAQKRQLENMEANVKSQYAYAAQNLLSHHDSHSSVILKMPIEGSENPERPFLEKADKTALKMRIPVAGGDKAASSKPEEIKMRIKVHAAADKHNSVEDSVTKSREHKEKHKTHPSNHHHHHNHHSHKHSHSQLPVGTGNKRPGDPKHSSQTSNLAHKTYSLSSSFSSSSSTRKRGPSEETGGAVFDHPAKIAKSTKSSSLNFSFPSLPTMGQMPGHSSDTSGLSFSQPSCKTRVPHSKLDKGPTGANGHNTTQTIDYQDTVNMLHSLLSAQGVQPTQPTAFEFVRPYSDYLNPRSGGISSRSGNTDKPRPPPLPSEPPPPLPPLPK

SEQ ID NO: 6 - DNA, 2181 bp, Artificial Sequence
  Cyclin T1 nucleic acid sequence
atggagggagagaggaagaacaacaacaaacggtggtatttcactcgagaacagctggaaaatagcccatcccgtcgttttggcgtggacccagataaagaactttcttatcgccagcaggcggccaatctgcttcaggacatggggcagcgtcttaacgtctcacaattgactatcaacactgctatagtatacatgcatcgattctacatgattcagtccttcacacagttccctggaaattctgtggctccagcagccttgtttctagcagctaaagtggaggagcagcccaaaaaattggaacatgtcatcaaggtagcacatacttgtctccatcctcaggaatcccttcctgatactagaagtgaggcttatttgcaacaagttcaagatctggtcattttagaaagcataattttgcagactttaggctttgaactaacaattgatcacccacatactcatgtagtaaagtgcactcaacttgttcgagcaagcaaggacttagcacagacttcttacttcatggcaaccaacagcctgcatttgaccacatttagcctgcagtacacacctcctgtggtggcctgtgtctgcattcacctggcttgcaagtggtccaattgggagatcccagtctcaactgacgggaagcactggtgggagtatgttgacgccactgtgaccttggaacttttagatgaactgacacatgagtttctacagattttggagaaaactcccaacaggctcaaacgcatttggaattggagggcatgcgaggctgccaagaaaacaaaagcagatgaccgaggaacagatgaaaagacttcagagcagacaatcctcaatatgatttcccagagctcttcagacacaaccattgcaggtttaatgagcatgtcaacttctaccacaagtgcagtgccttccctgccagtctccgaagagtcatccagcaacttaaccagtgtggagatgttgccgggcaagcgttggctgtcctcccaaccttctttcaaactagaacctactcagggtcatcggactagtgagaatttagcacttacaggagttgatcattccttaccacaggatggttcaaatgcatttatttcccagaagcagaatagtaagagtgtgccatcagctaaagtgtcactgaaagaataccgcgcgaagcatgcagaagaattggctgcccagaagaggcaactggagaacatggaagccaatgtgaagtcacaatatgcatatgctgcccagaatctcctttctcatcatgatagccattcttcagtcattctaaaaatgcccatagagggttcagaaaaccccgagcggccttttctggaaaaggctgacaaaacagctctcaaaatgagaatcccagtggcaggtggagataaagctgcgtcttcaaaaccagaggagataaaaatgcgcataaaagtccatgctgcagctgataagcacaattctgtagaggacagtgttacaaagagccgagagcacaaagaaaagcacaagactcacccatctaatcatcatcatcatcataatcaccactcacacaagcactctcattcccaacttccagttggtactgggaacaaacgtcctggtgatccaaaacatagtagccagacaagcaacttagcacataaaacctatagcttgtctagttctttttcctcttccagttctactcgtaaaaggggaccctctgaagagactggaggggctgtgtttgatcatccagccaagattgccaagagtactaaatcctcttccctaaatttctccttcccttcacttcctacaatgggtcagatgcctgggcatagctcagacacaagtggcctttccttttcacagcccagctgtaaaactcgtgtccctcattcgaaactggataaagggcccactggggccaatggtcacaacacgacccagacaatagactatcaagacactgtgaatatgcttcactccctgctcagtgcccagggtgttcagcccactcagcccactgcatttgaatttgttcgtccttatagtgactatctgaatcctcggtctggtggaatctcctcgagatctggcaatacagacaaaccccggccaccacctctgccatcagaacctcctccaccacttccaccccttcctaagtaa

SEQ ID NO: 7 - protein, 372 aa, Artificial Sequence
  CDK9 amino acid sequence
MAKQYDSVECPFCDEVSKYEKLAKIGQGTFGEVFKARHRKTGQKVALKKVLMENEKEGFPITALREIKILQLLKHENVVNLIEICRTKASPYNRCKGSIYLVFDFCEHDLAGLLSNVLVKFTLSEIKRVMQMLLNGLYYIHRNKILHRDMKAANVLITRDGVLKLADFGLARAFSLAKNSQPNRYTNRVVTLWYRPPELLLGERDYGPPIDLWGAGCIMAEMWTRSPIMQGNTEQHQLALISQLCGSITPEVWPNVDNYELYEKLELVKGQKRKVKDRLKAYVRDPYALDLIDKLLVLDPAQRIDSDDALNHDFFWSDPMPSDLKGMLSTHLTSMFEYLAPPRRKGSQITQQSTNQSRNPATTNQTEFERVF

SEQ ID NO: 8 - DNA, 1119 bp, Artificial Sequence
  CDK9 nucleic acid sequence
atggcaaagcagtacgactcggtggagtgccctttttgtgatgaagtttccaaatacgagaagctcgccaagatcggccaaggcaccttcggggaggtgttcaaggccaggcaccgcaagaccggccagaaggtggctctgaagaaggtgctgatggaaaacgagaaggaggggttccccattacagccttgcgggagatcaagatccttcagcttctaaaacacgagaatgtggtcaacttgattgagatttgtcgaaccaaagcttccccctataaccgctgcaagggtagtatatacctggtgttcgacttctgcgagcatgaccttgctgggctgttgagcaatgttttggtcaagttcacgctgtctgagatcaagagggtgatgcagatgctgcttaacggcctctactacatccacagaaacaagatcctgcatagggacatgaaggctgctaatgtgcttatcactcgtgatggggtcctgaagctggcagactttgggctggcccgggccttcagcctggccaagaacagccagcccaaccgctacaccaaccgtgtggtgacactctggtaccggcccccggagctgttgctcggggagcgggactacggcccccccattgacctgtggggtgctgggtgcatcatggcagagatgtggacccgcagccccatcatgcagggcaacacggagcagcaccaactcgccctcatcagtcagctctgcggctccatcacccctgaggtgtggccaaacgtggacaactatgagctgtacgaaaagctggagctggtcaagggccagaagcggaaggtgaaggacaggctgaaggcctatgtgcgtgacccatacgcactggacctcatcgacaagctgctggtgctggaccctgcccagcgcatcgacagcgatgacgccctcaaccacgacttcttctggtccgaccccatgccctccgacctcaagggcatgctctccacccacctgacgtccatgttcgagtacttggcaccaccgcgccggaagggcagccagatcacccagcagtccaccaaccagagtcgcaatcccgccaccaccaaccagacggagtttgagcgcgtcttctga

SEQ ID NO: 9 - DNA, 2291 bp, Artificial Sequence
  MycERTM
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacgggatccacgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgcttgctacgtcaagtcggttccgcatgatgaacctgcagggtgaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcacatgagtaacaaacgcatggagcatctctacaacatgaaatgcaagaacgtggtacccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctgagagctcctactagtgaattc

SEQ ID NO: 10 - protein, 762 aa, Artificial Sequence
  MycERTM
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRDPRNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKRMEHLYNMKCKNVVPLYDLLLEMLDAHRLHAPASRMGVPPEEPSQTQLATTSSTSAHSLQTYYIPPEAEGFPNTIELLLVN

SEQ ID NO: 11 - protein, 454 aa, Artificial Sequence
  c-myc
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 12 - DNA, 1365 bp, Artificial Sequence
  c-myc
ctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 13 - RNA, 2181 nt, Artificial Sequence
  Cyclin T1
auggagggagagaggaagaacaacaacaaacggugguauuucacucgagaacagcuggaaaauagcccaucccgucguuuuggcguggacccagauaaagaacuuucuuaucgccagcaggcggccaaucugcuucaggacauggggcagcgucuuaacgucucacaauugacuaucaacacugcuauaguauacaugcaucgauucuacaugauucaguccuucacacaguucccuggaaauucuguggcuccagcagccuuguuucuagcagcuaaaguggaggagcagcccaaaaaauuggaacaugucaucaagguagcacauacuugucuccauccucaggaaucccuuccugauacuagaagugaggcuuauuugcaacaaguucaagaucuggucauuuuagaaagcauaauuuugcagacuuuaggcuuugaacuaacaauugaucacccacauacucauguaguaaagugcacucaacuuguucgagcaagcaaggacuuagcacagacuucuuacuucauggcaaccaacagccugcauuugaccacauuuagccugcaguacacaccuccugugguggccugugucugcauucaccuggcuugcaagugguccaauugggagaucccagucucaacugacgggaagcacuggugggaguauguugacgccacugugaccuuggaacuuuuagaugaacugacacaugaguuucuacagauuuuggagaaaacucccaacaggcucaaacgcauuuggaauuggagggcaugcgaggcugccaagaaaacaaaagcagaugaccgaggaacagaugaaaagacuucagagcagacaauccucaauaugauuucccagagcucuucagacacaaccauugcagguuuaaugagcaugucaacuucuaccacaagugcagugccuucccugccagucuccgaagagucauccagcaacuuaaccaguguggagauguugccgggcaagcguuggcuguccucccaaccuucuuucaaacuagaaccuacucagggucaucggacuagugagaauuuagcacuuacaggaguugaucauuccuuaccacaggaugguucaaaugcauuuauuucccagaagcagaauaguaagagugugccaucagcuaaagugucacugaaagaauaccgcgcgaagcaugcagaagaauuggcugcccagaagaggcaacuggagaacauggaagccaaugugaagucacaauaugcauaugcugcccagaaucuccuuucucaucaugauagccauucuucagucauucuaaaaaugcccauagaggguucagaaaaccccgagcggccuuuucuggaaaaggcugacaaaacagcucucaaaaugagaaucccaguggcagguggagauaaagcugcgucuucaaaaccagaggagauaaaaaugcgcauaaaaguccaugcugcagcugauaagcacaauucuguagaggacaguguuacaaagagccgagagcacaaagaaaagcacaagacucacccaucuaaucaucaucaucaucauaaucaccacucacacaagcacucucauucccaacuuccaguugguacugggaacaaacguccuggugauccaaaacauaguagccagacaagcaacuuagcacauaaaaccuauagcuugucuaguucuuuuuccucuuccaguucuacucguaaaaggggacccucugaagagacuggaggggcuguguuugaucauccagccaagauugccaagaguacuaaauccucuucccuaaauuucuccuucccuucacuuccuacaaugggucagaugccugggcauagcucagacacaaguggccuuuccuuuucacagcccagcuguaaaacucgugucccucauucgaaacuggauaaagggcccacuggggccaauggucacaacacgacccagacaauagacuaucaagacacugugaauaugcuucacucccugcucagugcccaggguguucagcccacucagcccacugcauuugaauuuguucguccuuauagugacuaucugaauccucggucugguggaaucuccucgagaucuggcaauacagacaaaccccggccaccaccucugccaucagaaccuccuccaccacuuccaccccuuccuaaguaa

SEQ ID NO: 14 - RNA, 1119 nt, Artificial Sequence
  CDK9
auggcaaagcaguacgacucgguggagugcccuuuuugugaugaaguuuccaaauacgagaagcucgccaagaucggccaaggcaccuucggggagguguucaaggccaggcaccgcaagaccggccagaagguggcucugaagaaggugcugauggaaaacgagaaggagggguuccccauuacagccuugcgggagaucaagauccuucagcuucuaaaacacgagaauguggucaacuugauugagauuugucgaaccaaagcuucccccuauaaccgcugcaaggguaguauauaccugguguucgacuucugcgagcaugaccuugcugggcuguugagcaauguuuuggucaaguucacgcugucugagaucaagagggugaugcagaugcugcuuaacggccucuacuacauccacagaaacaagauccugcauagggacaugaaggcugcuaaugugcuuaucacucgugaugggguccugaagcuggcagacuuugggcuggcccgggccuucagccuggccaagaacagccagcccaaccgcuacaccaaccguguggugacacucugguaccggcccccggagcuguugcucggggagcgggacuacggcccccccauugaccuguggggugcugggugcaucauggcagagauguggacccgcagccccaucaugcagggcaacacggagcagcaccaacucgcccucaucagucagcucugcggcuccaucaccccugagguguggccaaacguggacaacuaugagcuguacgaaaagcuggagcuggucaagggccagaagcggaaggugaaggacaggcugaaggccuaugugcgugacccauacgcacuggaccucaucgacaagcugcuggugcuggacccugcccagcgcaucgacagcgaugacgcccucaaccacgacuucuucugguccgaccccaugcccuccgaccucaagggcaugcucuccacccaccugacguccauguucgaguacuuggcaccaccgcgccggaagggcagccagaucacccagcaguccaccaaccagagucgcaaucccgccaccaccaaccagacggaguuugagcgcgucuucuga

SEQ ID NO: 15 - RNA, 1365 nt, Artificial Sequence
  c-myc
cuggauuuuuuucggguaguggaaaaccagcagccucccgcgacgaugccccucaacguuagcuucaccaacaggaacuaugaccucgacuacgacucggugcagccguauuucuacugcgacgaggaggagaacuucuaccagcagcagcagcagagcgagcugcagcccccggcgcccagcgaggauaucuggaagaaauucgagcugcugcccaccccgccccuguccccuagccgccgcuccgggcucugcucgcccuccuacguugcggucacacccuucucccuucggggagacaacgacggcgguggcgggagcuucuccacggccgaccagcuggagauggugaccgagcugcugggaggagacauggugaaccagaguuucaucugcgacccggacgacgagaccuucaucaaaaacaucaucauccaggacuguauguggagcggcuucucggccgccgccaagcucgucucagagaagcuggccuccuaccaggcugcgcgcaaagacagcggcagcccgaaccccgcccgcggccacagcgucugcuccaccuccagcuuguaccugcaggaucugagcgccgccgccucagagugcaucgaccccucgguggucuuccccuacccucucaacgacagcagcucgcccaaguccugcgccucgcaagacuccagcgccuucucuccguccucggauucucugcucuccucgacggaguccuccccgcagggcagccccgagccccuggugcuccaugaggagacaccgcccaccaccagcagcgacucugaggaggaacaagaagaugaggaagaaaucgauguuguuucuguggaaaagaggcaggcuccuggcaaaaggucagagucuggaucaccuucugcuggaggccacagcaaaccuccucacagcccacugguccucaagaggugccacgucuccacacaucagcacaacuacgcagcgccucccuccacucggaaggacuauccugcugccaagagggucaaguuggacagugucagaguccugagacagaucagcaacaaccgaaaaugcaccagccccagguccucggacaccgaggagaaugucaagaggcgaacacacaacgucuuggagcgccagaggaggaacgagcuaaaacggagcuuuuuugcccugcgugaccagaucccggaguuggaaaacaaugaaaaggcccccaagguaguuauccuuaaaaaagccacagcauacauccuguccguccaagcagaggagcaaaagcucauuucugaagaggacuuguugcggaaacgacgagaacaguugaaacacaaacuugaacagcuacggaacucuugugcguaa

SEQ ID NO: 16 - RNA, 2181 nt, Artificial Sequence
  Cyclin T1 - pseudouridine* (Denoted by P)
  Pseudouridine (1)..(2181): n denotes pseudouridine
  misc_feature (2)..(2): n is a, c, g, or u
  misc_feature (34)..(34): n is a, c, g, or u
  misc_feature (37)..(37): n is a, c, g, or u
  misc_feature (39)..(41): n is a, c, g, or u
  misc_feature (45)..(45): n is a, c, g, or u
  misc_feature (56)..(56): n is a, c, g, or u
  misc_feature (63)..(63): n is a, c, g, or u
  misc_feature (70)..(70): n is a, c, g, or u
  misc_feature (75)..(75): n is a, c, g, or u
  misc_feature (78)..(81): n is a, c, g, or u
  misc_feature (86)..(86): n is a, c, g, or u
  misc_feature (96)..(96): n is a, c, g, or u
  misc_feature (104)..(106): n is a, c, g, or u
  misc_feature (108)..(109): n is a, c, g, or u
  misc_feature (111)..(111): n is a, c, g, or u
  misc_feature (129)..(129): n is a, c, g, or u
  misc_feature (131)..(131): n is a, c, g, or u
  misc_feature (134)..(135): n is a, c, g, or u
  misc_feature (143)..(143): n is a, c, g, or u
  misc_feature (153)..(153): n is a, c, g, or u
  misc_feature (155)..(156): n is a, c, g, or u
  misc_feature (161)..(161): n is a, c, g, or u
  misc_feature (163)..(163): n is a, c, g, or u
  misc_feature (169)..(170): n is a, c, g, or u
  misc_feature (174)..(174): n is a, c, g, or u
  misc_feature (176)..(176): n is a, c, g, or u
  misc_feature (183)..(183): n is a, c, g, or u
  misc_feature (186)..(186): n is a, c, g, or u
  misc_feature (188)..(188): n is a, c, g, or u
  misc_feature (191)..(191): n is a, c, g, or u
  misc_feature (193)..(193): n is a, c, g, or u
  misc_feature (197)..(197): n is a, c, g, or u
  misc_feature (201)..(201): n is a, c, g, or u
  misc_feature (205)..(206): n is a, c, g, or u
  misc_feature (208)..(208): n is a, c, g, or u
  misc_feature (212)..(212): n is a, c, g, or u
  misc_feature (215)..(216): n is a, c, g, or u
  misc_feature (220)..(220): n is a, c, g, or u
  misc_feature (223)..(224): n is a, c, g, or u
  misc_feature (232)..(233): n is a, c, g, or u
  misc_feature (237)..(237): n is a, c, g, or u
  misc_feature (243)..(244): n is a, c, g, or u
  misc_feature (246)..(246): n is a, c, g, or u
  misc_feature (248)..(248): n is a, c, g, or u
  misc_feature (252)..(252): n is a, c, g, or u
  misc_feature (262)..(263): n is a, c, g, or u
  misc_feature (265)..(267): n is a, c, g, or u
  misc_feature (269)..(269): n is a, c, g, or u
  misc_feature (276)..(276): n is a, c, g, or u
  misc_feature (281)..(281): n is a, c, g, or u
  misc_feature (301)..(302): n is a, c, g, or u
  misc_feature (309)..(309): n is a, c, g, or u
  misc_feature (311)..(311): n is a, c, g, or u
  misc_feature (314)..(314): n is a, c, g, or u
  misc_feature (320)..(320): n is a, c, g, or u
  misc_feature (327)..(327): n is a, c, g, or u
  misc_feature (330)..(331): n is a, c, g, or u
  misc_feature (333)..(333): n is a, c, g, or u
  misc_feature (335)..(335): n is a, c, g, or u
  misc_feature (339)..(339): n is a, c, g, or u
  misc_feature (342)..(342): n is a, c, g, or u
  misc_feature (349)..(349): n is a, c, g, or u
  misc_feature (353)..(354): n is a, c, g, or u
  misc_feature (357)..(357): n is a, c, g, or u
  misc_feature (360)..(360): n is a, c, g, or u
  misc_feature (363)..(363): n is a, c, g, or u
  misc_feature (369)..(369): n is a, c, g, or u
  misc_feature (375)..(376): n is a, c, g, or u
  misc_feature (378)..(380): n is a, c, g, or u
  misc_feature (389)..(390): n is a, c, g, or u
  misc_feature (396)..(396): n is a, c, g, or u
  misc_feature (398)..(398): n is a, c, g, or u
  misc_feature (401)..(401): n is a, c, g, or u
  misc_feature (404)..(407): n is a, c, g, or u
  misc_feature (416)..(416): n is a, c, g, or u
  misc_feature (419)..(422): n is a, c, g, or u
  misc_feature (429)..(431): n is a, c, g, or u
  misc_feature (436)..(438): n is a, c, g, or u
  misc_feature (443)..(443): n is a, c, g, or u
  misc_feature (449)..(450): n is a, c, g, or u
  misc_feature (453)..(453): n is a, c, g, or u
  misc_feature (462)..(462): n is a, c, g, or u
  misc_feature (465)..(465): n is a, c, g, or u
  misc_feature (468)..(468): n is a, c, g, or u
  misc_feature (470)..(470): n is a, c, g, or u
  misc_feature (473)..(473): n is a, c, g, or u
  misc_feature (478)..(478): n is a, c, g, or u
  misc_feature (483)..(483): n is a, c, g, or u
  misc_feature (488)..(489): n is a, c, g, or u
  misc_feature (491)..(492): n is a, c, g, or u
  misc_feature (508)..(509): n is a, c, g, or u
  misc_feature (519)..(520): n is a, c, g, or u
  misc_feature (522)..(523): n is a, c, g, or u
  misc_feature (526)..(527): n is a, c, g, or u
  misc_feature (530)..(530): n is a, c, g, or u
  misc_feature (545)..(545): n is a, c, g, or u
  misc_feature (549)..(551): n is a, c, g, or u
  misc_feature (559)..(561): n is a, c, g, or u
  misc_feature (566)..(566): n is a, c, g, or u
  misc_feature (571)..(571): n is a, c, g, or u
  misc_feature (579)..(579): n is a, c, g, or u
  misc_feature (582)..(582): n is a, c, g, or u
  misc_feature (584)..(584): n is a, c, g, or u
  misc_feature (587)..(587): n is a, c, g, or u
  misc_feature (592)..(592): n is a, c, g, or u
  misc_feature (594)..(594): n is a, c, g, or u
  misc_feature (596)..(596): n is a, c, g, or u
  misc_feature (598)..(598): n is a, c, g, or u
  misc_feature (602)..(603): n is a, c, g, or u
  misc_feature (608)..(608): n is a, c, g, or u
  misc_feature (612)..(613): n is a, c, g, or u
  misc_feature (619)..(619): n is a, c, g, or u
  misc_feature (622)..(622): n is a, c, g, or u
  misc_feature (627)..(628): n is a, c, g, or u
  misc_feature (635)..(635): n is a, c, g, or u
  misc_feature (641)..(641): n is a, c, g, or u
  misc_feature (643)..(643): n is a, c, g, or u
  misc_feature (648)..(648): n is a, c, g, or u
  misc_feature (661)..(661): n is a, c, g, or u
  misc_feature (664)..(664): n is a, c, g, or u
  misc_feature (670)..(670): n is a, c, g, or u
  misc_feature (672)..(672): n is a, c, g, or u
  misc_feature (674)..(675): n is a, c, g, or u
  misc_feature (684)..(684): n is a, c, g, or u
  misc_feature (686)..(686): n is a, c, g, or u
  misc_feature (691)..(692): n is a, c, g, or u
  misc_feature (698)..(701): n is a, c, g, or u
  misc_feature (705)..(705): n is a, c, g, or u
  misc_feature (710)..(710): n is a, c, g, or u
  misc_feature (717)..(717): n is a, c, g, or u
  misc_feature (721)..(723): n is a, c, g, or u
  misc_feature (725)..(725): n is a, c, g, or u
  misc_feature (731)..(734): n is a, c, g, or u
  misc_feature (744)..(744): n is a, c, g, or u
  misc_feature (755)..(755): n is a, c, g, or u
  misc_feature (764)..(766): n is a, c, g, or u
  misc_feature (771)..(772): n is a, c, g, or u
  misc_feature (781)..(781): n is a, c, g, or u
  misc_feature (789)..(789): n is a, c, g, or u
  misc_feature (810)..(810): n is a, c, g, or u
  misc_feature (825)..(825): n is a, c, g, or u
  misc_feature (834)..(835): n is a, c, g, or u
  misc_feature (848)..(848): n is a, c, g, or u
  misc_feature (851)..(851): n is a, c, g, or u
  misc_feature (855)..(855): n is a, c, g, or u
  misc_feature (857)..(857): n is a, c, g, or u
  misc_feature (860)..(862): n is a, c, g, or u
  misc_feature (871)..(871): n is a, c, g, or u
  misc_feature (873)..(874): n is a, c, g, or u
  misc_feature (887)..(888): n is a, c, g, or u
  misc_feature (894)..(896): n is a, c, g, or u
  misc_feature (899)..(899): n is a, c, g, or u
  misc_feature (905)..(905): n is a, c, g, or u
  misc_feature (907)..(907): n is a, c, g, or u
  misc_feature (912)..(913): n is a, c, g, or u
  misc_feature (915)..(915): n is a, c, g, or u
  misc_feature (924)..(924): n is a, c, g, or u
  misc_feature (929)..(929): n is a, c, g, or u
  misc_feature (933)..(934): n is a, c, g, or u
  misc_feature (938)..(938): n is a, c, g, or u
  misc_feature (944)..(944): n is a, c, g, or u
  misc_feature (946)..(946): n is a, c, g, or u
  misc_feature (955)..(955): n is a, c, g, or u
  misc_feature (958)..(958): n is a, c, g, or u
  misc_feature (967)..(968): n is a, c, g, or u
  misc_feature (975)..(975): n is a, c, g, or u
  misc_feature (977)..(977): n is a, c, g, or u
  misc_feature (983)..(983): n is a, c, g, or u
  misc_feature (985)..(986): n is a, c, g, or u
  misc_feature (999)..(1000): n is a, c, g, or u
  misc_feature (1004)..(1004): n is a, c, g, or u
  misc_feature (1006)..(1006): n is a, c, g, or u
  misc_feature (1009)..(1009): n is a, c, g, or u
  misc_feature (1017)..(1018): n is a, c, g, or u
  misc_feature (1020)..(1022): n is a, c, g, or u
  misc_feature (1028)..(1028): n is a, c, g, or u
  misc_feature (1035)..(1035): n is a, c, g, or u
  misc_feature (1038)..(1038): n is a, c, g, or u
  misc_feature (1044)..(1044): n is a, c, g, or u
  misc_feature (1047)..(1047): n is a, c, g, or u
  misc_feature (1053)..(1053): n is a, c, g, or u
  misc_feature (1056)..(1056): n is a, c, g, or u
  misc_feature (1062)..(1064): n is a, c, g, or u
  misc_feature (1070)..(1071): n is a, c, g, or u
  misc_feature (1079)..(1080): n is a, c, g, or u
  misc_feature (1083)..(1083): n is a, c, g, or u
  misc_feature (1086)..(1087): n is a, c, g, or u
  misc_feature (1090)..(1091): n is a, c, g, or u
  misc_feature (1101)..(1101): n is a, c, g, or u
  misc_feature (1104)..(1105): n is a, c, g, or u
  misc_feature (1110)..(1110): n is a, c, g, or u
  misc_feature (1114)..(1116): n is a, c, g, or u
  misc_feature (1118)..(1120): n is a, c, g, or u
  misc_feature (1134)..(1134): n is a, c, g, or u
  misc_feature (1137)..(1137): n is a, c, g, or u
  misc_feature (1143)..(1143): n is a, c, g, or u
  misc_feature (1145)..(1145): n is a, c, g, or u
  misc_feature (1150)..(1150): n is a, c, g, or u
  misc_feature (1155)..(1155): n is a, c, g, or u
  misc_feature (1160)..(1160): n is a, c, g, or u
  misc_feature (1162)..(1162): n is a, c, g, or u
  misc_feature (1166)..(1166): n is a, c, g, or u
  misc_feature (1174)..(1174): n is a, c, g, or u
  misc_feature (1188)..(1188): n is a, c, g, or u
  misc_feature (1198)..(1199): n is a, c, g, or u
  misc_feature (1203)..(1203): n is a, c, g, or u
  misc_feature (1220)..(1220): n is a, c, g, or u
  misc_feature (1229)..(1229): n is a, c, g, or u
  misc_feature (1239)..(1239): n is a, c, g, or u
  misc_feature (1241)..(1241): n is a, c, g, or u
  misc_feature (1246)..(1246): n is a, c, g, or u
  misc_feature (1252)..(1252): n is a, c, g, or u
  misc_feature (1254)..(1254): n is a, c, g, or u
  misc_feature (1258)..(1258): n is a, c, g, or u
  misc_feature (1260)..(1260): n is a, c, g, or u
  misc_feature (1263)..(1263): n is a, c, g, or u
  misc_feature (1272)..(1272): n is a, c, g, or u
  misc_feature (1274)..(1274): n is a, c, g, or u
  misc_feature (1277)..(1279): n is a, c, g, or u
  misc_feature (1281)..(1281): n is a, c, g, or u
  misc_feature (1284)..(1284): n is a, c, g, or u
  misc_feature (1287)..(1287): n is a, c, g, or u
  misc_feature (1290)..(1290): n is a, c, g, or u
  misc_feature (1296)..(1297): n is a, c, g, or u
  misc_feature (1299)..(1300): n is a, c, g, or u
  misc_feature (1304)..(1304): n is a, c, g, or u
  misc_feature (1307)..(1308): n is a, c, g, or u
  misc_feature (1310)..(1310): n is a, c, g, or u
  misc_feature (1316)..(1316): n is a, c, g, or u
  misc_feature (1322)..(1322): n is a, c, g, or u
  misc_feature (1329)..(1330): n is a, c, g, or u
  misc_feature (1350)..(1353): n is a, c, g, or u
  misc_feature (1355)..(1355): n is a, c, g, or u
  misc_feature (1365)..(1365): n is a, c, g, or u
  misc_feature (1377)..(1377): n is a, c, g, or u
  misc_feature (1379)..(1379): n is a, c, g, or u
  misc_feature (1385)..(1385): n is a, c, g, or u
  misc_feature (1391)..(1391): n is a, c, g, or u
  misc_feature (1397)..(1397): n is a, c, g, or u
  misc_feature (1404)..(1404): n is a, c, g, or u
  misc_feature (1410)..(1410): n is a, c, g, or u
  misc_feature (1416)..(1416): n is a, c, g, or u
  misc_feature (1420)..(1420): n is a, c, g, or u
  misc_feature (1422)..(1423): n is a, c, g, or u
  misc_feature (1439)..(1439): n is a, c, g, or u
  misc_feature (1445)..(1445): n is a, c, g, or u
  misc_feature (1451)..(1451): n is a, c, g, or u
  misc_feature (1457)..(1457): n is a, c, g, or u
  misc_feature (1461)..(1461): n is a, c, g, or u
  misc_feature (1464)..(1464): n is a, c, g, or u
  misc_feature (1470)..(1470): n is a, c, g, or u
  misc_feature (1473)..(1473): n is a, c, g, or u
  misc_feature (1482)..(1483): n is a, c, g, or u
  misc_feature (1485)..(1485): n is a, c, g, or u
  misc_feature (1487)..(1487): n is a, c, g, or u
  misc_feature (1497)..(1497): n is a, c, g, or u
  misc_feature (1499)..(1500): n is a, c, g, or u
  misc_feature (1536)..(1536): n is a, c, g, or u
  misc_feature (1543)..(1543): n is a, c, g, or u
  misc_feature (1545)..(1545): n is a, c, g, or u
  misc_feature (1548)..(1548): n is a, c, g, or u
  misc_feature (1551)..(1551): n is a, c, g, or u
  misc_feature (1554)..(1554): n is a, c, g, or u
  misc_feature (1557)..(1557): n is a, c, g, or u
  misc_feature (1560)..(1560): n is a, c, g, or u
  misc_feature (1563)..(1563): n is a, c, g, or u
  misc_feature (1566)..(1566): n is a, c, g, or u
  misc_feature (1573)..(1573): n is a, c, g, or u
  misc_feature (1585)..(1585): n is a, c, g, or u
  misc_feature (1587)..(1587): n is a, c, g, or u
  misc_feature (1590)..(1591): n is a, c, g, or u
  misc_feature (1598)..(1599): n is a, c, g, or u
  misc_feature (1604)..(1605): n is a, c, g, or u
  misc_feature (1608)..(1608): n is a, c, g, or u
  misc_feature (1611)..(1611): n is a, c, g, or u
  misc_feature (1623)..(1623): n is a, c, g, or u
  misc_feature (1626)..(1626): n is a, c, g, or u
  misc_feature (1629)..(1629): n is a, c, g, or u
  misc_feature (1632)..(1632): n is a, c, g, or u
  misc_feature (1641)..(1641): n is a, c, g, or u
  misc_feature (1644)..(1644): n is a, c, g, or u
  misc_feature (1660)..(1661): n is a, c, g, or u
  misc_feature (1668)..(1668): n is a, c, g, or u
  misc_feature (1675)..(1675): n is a, c, g, or u
  misc_feature (1677)..(1677): n is a, c, g, or u
  misc_feature (1681)..(1682): n is a, c, g, or u
  misc_feature (1684)..(1684): n is a, c, g, or u
  misc_feature (1686)..(1686): n is a, c, g, or u
  misc_feature (1689)..(1690): n is a, c, g, or u
  misc_feature (1692)..(1696): n is a, c, g, or u
  misc_feature (1699)..(1699): n is a, c, g, or u
  misc_feature (1701)..(1702): n is a, c, g, or u
  misc_feature (1707)..(1708): n is a, c, g, or u
  misc_feature (1710)..(1710): n is a, c, g, or u
  misc_feature (1713)..(1713): n is a, c, g, or u
  misc_feature (1716)..(1716): n is a, c, g, or u
  misc_feature (1729)..(1729): n is a, c, g, or u
  misc_feature (1731)..(1731): n is a, c, g, or u
  misc_feature (1740)..(1740): n is a, c, g, or u
  misc_feature (1749)..(1749): n is a, c, g, or u
  misc_feature (1751)..(1751): n is a, c, g, or u
  misc_feature (1753)..(1755): n is a, c, g, or u
  misc_feature (1758)..(1758): n is a, c, g, or u
  misc_feature (1761)..(1761): n is a, c, g, or u
  misc_feature (1772)..(1773): n is a, c, g, or u
  misc_feature (1782)..(1782): n is a, c, g, or u
  misc_feature (1785)..(1785): n is a, c, g, or u
  misc_feature (1789)..(1789): n is a, c, g, or u
  misc_feature (1792)..(1792): n is a, c, g, or u
  misc_feature (1794)..(1795): n is a, c, g, or u
  misc_feature (1799)..(1799): n is a, c, g, or u
  misc_feature (1803)..(1805): n is a, c, g, or u
  misc_feature (1807)..(1807): n is a, c, g, or u
  misc_feature (1810)..(1811): n is a, c, g, or u
  misc_feature (1815)..(1816): n is a, c, g, or u
  misc_feature (1820)..(1821): n is a, c, g, or u
  misc_feature (1824)..(1824): n is a, c, g, or u
  misc_feature (1829)..(1829): n is a, c, g, or u
  misc_feature (1833)..(1833): n is a, c, g, or u
  misc_feature (1838)..(1838): n is a, c, g, or u
  misc_feature (1842)..(1842): n is a, c, g, or u
  misc_feature (1848)..(1848): n is a, c, g, or u
  misc_feature (1852)..(1852): n is a, c, g, or u
  misc_feature (1863)..(1863): n is a, c, g, or u
  misc_feature (1868)..(1870): n is a, c, g, or u
  misc_feature (1873)..(1876): n is a, c, g, or u
  misc_feature (1888)..(1888): n is a, c, g, or u
  misc_feature (1890)..(1890): n is a, c, g, or u
  misc_feature (1896)..(1896): n is a, c, g, or u
  misc_feature (1899)..(1899): n is a, c, g, or u
  misc_feature (1901)..(1901): n is a, c, g, or u
  misc_feature (1905)..(1905): n is a, c, g, or u
  misc_feature (1908)..(1909): n is a, c, g, or u
  misc_feature (1916)..(1916): n is a, c, g, or u
  misc_feature (1920)..(1920): n is a, c, g, or u
  misc_feature (1932)..(1932): n is a, c, g, or u
  misc_feature (1941)..(1941): n is a, c, g, or u
  misc_feature (1944)..(1944): n is a, c, g, or u
  misc_feature (1964)..(1964): n is a, c, g, or u
  misc_feature (1969)..(1969): n is a, c, g, or u
  misc_feature (1971)..(1971): n is a, c, g, or u
  misc_feature (1980)..(1980): n is a, c, g, or u
  misc_feature (1982)..(1982): n is a, c, g, or u
  misc_feature (1986)..(1986): n is a, c, g, or u
  misc_feature (1988)..(1988): n is a, c, g, or u
  misc_feature (1991)..(1992): n is a, c, g, or u
  misc_feature (1996)..(1996): n is a, c, g, or u
  misc_feature (2000)..(2000): n is a, c, g, or u
  misc_feature (2003)..(2003): n is a, c, g, or u
  misc_feature (2007)..(2007): n is a, c, g, or u
  misc_feature (2016)..(2016): n is a, c, g, or u
  misc_feature (2018)..(2019): n is a, c, g, or u
  misc_feature (2028)..(2028): n is a, c, g, or u
  misc_feature (2037)..(2037): n is a, c, g, or u
  misc_feature (2041)..(2043): n is a, c, g, or u
  misc_feature (2047)..(2049): n is a, c, g, or u
  misc_feature (2051)..(2052): n is a, c, g, or u
  misc_feature (2055)..(2055): n is a, c, g, or u
  misc_feature (2058)..(2059): n is a, c, g, or u
  misc_feature (2061)..(2061): n is a, c, g, or u
  misc_feature (2064)..(2064): n is a, c, g, or u
  misc_feature (2068)..(2068): n is a, c, g, or u
  misc_feature (2070)..(2070): n is a, c, g, or u
  misc_feature (2072)..(2072): n is a, c, g, or u
  misc_feature (2076)..(2076): n is a, c, g, or u
  misc_feature (2079)..(2079): n is a, c, g, or u
  misc_feature (2083)..(2083): n is a, c, g, or u
  misc_feature (2085)..(2085): n is a, c, g, or u
  misc_feature (2088)..(2088): n is a, c, g, or u
  misc_feature (2093)..(2093): n is a, c, g, or u
  misc_feature (2095)..(2095): n is a, c, g, or u
  misc_feature (2098)..(2098): n is a, c, g, or u
  misc_feature (2104)..(2104): n is a, c, g, or u
  misc_feature (2106)..(2106): n is a, c, g, or u
  misc_feature (2112)..(2112): n is a, c, g, or u
  misc_feature (2136)..(2136): n is a, c, g, or u
  misc_feature (2138)..(2138): n is a, c, g, or u
  misc_feature (2143)..(2143): n is a, c, g, or u
  misc_feature (2151)..(2151): n is a, c, g, or u
  misc_feature (2154)..(2154): n is a, c, g, or u
  misc_feature (2162)..(2163): n is a, c, g, or u
  misc_feature (2171)..(2172): n is a, c, g, or u
  misc_feature (2175)..(2175): n is a, c, g, or u
  misc_feature (2179)..(2179): n is a, c, g, or u
anggagggagagaggaagaacaacaacaaacggnggnannncacncgagaacagcnggaaaanagcccancccgncgnnnnggcgnggacccaganaaagaacnnncnnancgccagcaggcggccaancngcnncaggacanggggcagcgncnnaacgncncacaanngacnancaacacngcnanagnanacangcancganncnacanganncagnccnncacacagnncccnggaaanncngnggcnccagcagccnngnnncnagcagcnaaagnggaggagcagcccaaaaaannggaacangncancaaggnagcacanacnngncnccanccncaggaancccnnccnganacnagaagngaggcnnannngcaacaagnncaagancnggncannnnagaaagcanaannnngcagacnnnaggcnnngaacnaacaanngancacccacanacncangnagnaaagngcacncaacnngnncgagcaagcaaggacnnagcacagacnncnnacnncanggcaaccaacagccngcannngaccacannnagccngcagnacacaccnccngnggnggccngngncngcanncaccnggcnngcaagnggnccaanngggagancccagncncaacngacgggaagcacnggngggagnangnngacgccacngngaccnnggaacnnnnagangaacngacacangagnnncnacagannnnggagaaaacncccaacaggcncaaacgcannnggaannggagggcangcgaggcngccaagaaaacaaaagcagangaccgaggaacagangaaaagacnncagagcagacaanccncaanangannncccagagcncnncagacacaaccanngcaggnnnaangagcangncaacnncnaccacaagngcagngccnncccngccagncnccgaagagncanccagcaacnnaaccagngnggagangnngccgggcaagcgnnggcngnccncccaaccnncnnncaaacnagaaccnacncagggncancggacnagngagaannnagcacnnacaggagnngancannccnnaccacagganggnncaaangcannnannncccagaagcagaanagnaagagngngccancagcnaaagngncacngaaagaanaccgcgcgaagcangcagaagaannggcngcccagaagaggcaacnggagaacanggaagccaangngaagncacaanangcanangcngcccagaancnccnnncncancanganagccanncnncagncanncnaaaaangcccanagagggnncagaaaaccccgagcggccnnnncnggaaaaggcngacaaaacagcncncaaaangagaancccagnggcaggnggaganaaagcngcgncnncaaaaccagaggaganaaaaangcgcanaaaagnccangcngcagcnganaagcacaanncngnagaggacagngnnacaaagagccgagagcacaaagaaaagcacaagacncacccancnaancancancancancanaancaccacncacacaagcacncncanncccaacnnccagnnggnacngggaacaaacgnccnggnganccaaaacanagnagccagacaagcaacnnagcacanaaaaccnanagcnngncnagnncnnnnnccncnnccagnncnacncgnaaaaggggacccncngaagagacnggaggggcngngnnngancanccagccaaganngccaagagnacnaaanccncnncccnaaannncnccnncccnncacnnccnacaangggncagangccngggcanagcncagacacaagnggccnnnccnnnncacagcccagcngnaaaacncgngncccncanncgaaacngganaaagggcccacnggggccaanggncacaacacgacccagacaanagacnancaagacacngngaanangcnncacncccngcncagngcccagggngnncagcccacncagcccacngcannngaannngnncgnccnnanagngacnancngaanccncggncnggnggaancnccncgagancnggcaanacagacaaaccccggccaccaccncngccancagaaccnccnccaccacnnccaccccnnccnaagnaa

SEQ ID NO: 17 - RNA, 1119 nt, Artificial Sequence
  CDK9 - pseudouridine* (Denoted by P)
  Pseudouridine (1)..(1119): n denotes pseudouridine
  misc_feature (2)..(2): n is a, c, g, or u
  misc_feature (13)..(13): n is a, c, g, or u
  misc_feature (19)..(19): n is a, c, g, or u
  misc_feature (23)..(23): n is a, c, g, or u
  misc_feature (28)..(28): n is a, c, g, or u
  misc_feature (33)..(37): n is a, c, g, or u
  misc_feature (39)..(39): n is a, c, g, or u
  misc_feature (42)..(42): n is a, c, g, or u
  misc_feature (47)..(49): n is a, c, g, or u
  misc_feature (55)..(55): n is a, c, g, or u
  misc_feature (65)..(65): n is a, c, g, or u
  misc_feature (74)..(74): n is a, c, g, or u
  misc_feature (88)..(89): n is a, c, g, or u
  misc_feature (98)..(98): n is a, c, g, or u
  misc_feature (100)..(101): n is a, c, g, or u
  misc_feature (134)..(134): n is a, c, g, or u
  misc_feature (138)..(138): n is a, c, g, or u
  misc_feature (140)..(140): n is a, c, g, or u
  misc_feature (149)..(149): n is a, c, g, or u
  misc_feature (152)..(152): n is a, c, g, or u
  misc_feature (155)..(155): n is a, c, g, or u
  misc_feature (175)..(176): n is a, c, g, or u
  misc_feature (182)..(183): n is a, c, g, or u
  misc_feature (190)..(191): n is a, c, g, or u
  misc_feature (200)..(200): n is a, c, g, or u
  misc_feature (206)..(206): n is a, c, g, or u
  misc_feature (209)..(210): n is a, c, g, or u
  misc_feature (215)..(216): n is a, c, g, or u
  misc_feature (218)..(218): n is a, c, g, or u
  misc_feature (231)..(231): n is a, c, g, or u
  misc_feature (233)..(233): n is a, c, g, or u
  misc_feature (236)..(236): n is a, c, g, or u
  misc_feature (241)..(242): n is a, c, g, or u
  misc_feature (245)..(246): n is a, c, g, or u
  misc_feature (251)..(253): n is a, c, g, or u
  misc_feature (255)..(255): n is a, c, g, or u
  misc_feature (267)..(268): n is a, c, g, or u
  misc_feature (274)..(274): n is a, c, g, or u
  misc_feature (276)..(276): n is a, c, g, or u
  misc_feature (283)..(283): n is a, c, g, or u
  misc_feature (291)..(291): n is a, c, g, or u
  misc_feature (294)..(294): n is a, c, g, or u
  misc_feature (296)..(296): n is a, c, g, or u
  misc_feature (298)..(298): n is a, c, g, or u
  misc_feature (302)..(302): n is a, c, g, or u
  misc_feature (305)..(305): n is a, c, g, or u
  misc_feature (307)..(308): n is a, c, g, or u
  misc_feature (313)..(314): n is a, c, g, or u
  misc_feature (316)..(316): n is a, c, g, or u
  misc_feature (324)..(324): n is a, c, g, or u
  misc_feature (329)..(330): n is a, c, g, or u
  misc_feature (333)..(333): n is a, c, g, or u
  misc_feature (338)..(338): n is a, c, g, or u
  misc_feature (340)..(341): n is a, c, g, or u
  misc_feature (348)..(348): n is a, c, g, or u
  misc_feature (350)..(353): n is a, c, g, or u
  misc_feature (356)..(356): n is a, c, g, or u
  misc_feature (361)..(362): n is a, c, g, or u
  misc_feature (368)..(368): n is a, c, g, or u
  misc_feature (370)..(370): n is a, c, g, or u
  misc_feature (372)..(372): n is a, c, g, or u
  misc_feature (377)..(377): n is a, c, g, or u
  misc_feature (386)..(386): n is a, c, g, or u
  misc_feature (389)..(389): n is a, c, g, or u
  misc_feature (395)..(395): n is a, c, g, or u
  misc_feature (398)..(398): n is a, c, g, or u
  misc_feature (401)..(402): n is a, c, g, or u
  misc_feature (410)..(410): n is a, c, g, or u
  misc_feature (412)..(412): n is a, c, g, or u
  misc_feature (415)..(415): n is a, c, g, or u
  misc_feature (419)..(419): n is a, c, g, or u
  misc_feature (434)..(434): n is a, c, g, or u
  misc_feature (437)..(437): n is a, c, g, or u
  misc_feature (441)..(441): n is a, c, g, or u
  misc_feature (449)..(449): n is a, c, g, or u
  misc_feature (456)..(456): n is a, c, g, or u
  misc_feature (459)..(459): n is a, c, g, or u
  misc_feature (462)..(462): n is a, c, g, or u
  misc_feature (464)..(464): n is a, c, g, or u
  misc_feature (467)..(468): n is a, c, g, or u
  misc_feature (470)..(470): n is a, c, g, or u
  misc_feature (474)..(474): n is a, c, g, or u
  misc_feature (477)..(477): n is a, c, g, or u
  misc_feature (480)..(480): n is a, c, g, or u
  misc_feature (485)..(485): n is a, c, g, or u
  misc_feature (488)..(488): n is a, c, g, or u
  misc_feature (494)..(494): n is a, c, g, or u
  misc_feature (502)..(504): n is a, c, g, or u
  misc_feature (509)..(509): n is a, c, g, or u
  misc_feature (520)..(521): n is a, c, g, or u
  misc_feature (527)..(527): n is a, c, g, or u
  misc_feature (553)..(553): n is a, c, g, or u
  misc_feature (564)..(564): n is a, c, g, or u
  misc_feature (566)..(566): n is a, c, g, or u
  misc_feature (569)..(569): n is a, c, g, or u
  misc_feature (575)..(575): n is a, c, g, or u
  misc_feature (577)..(577): n is a, c, g, or u
  misc_feature (580)..(580): n is a, c, g, or u
  misc_feature (596)..(596): n is a, c, g, or u
  misc_feature (598)..(599): n is a, c, g, or u
  misc_feature (602)..(602): n is a, c, g, or u
  misc_feature (616)..(616): n is a, c, g, or u
  misc_feature (629)..(630): n is a, c, g, or u
  misc_feature (635)..(635): n is a, c, g, or u
  misc_feature (637)..(637): n is a, c, g, or u
  misc_feature (642)..(642): n is a, c, g, or u
  misc_feature (645)..(645): n is a, c, g, or u
  misc_feature (649)..(649): n is a, c, g, or u
  misc_feature (653)..(653): n is a, c, g, or u
  misc_feature (656)..(656): n is a, c, g, or u
  misc_feature (665)..(665): n is a, c, g, or u
  misc_feature (667)..(667): n is a, c, g, or u
  misc_feature (683)..(683): n is a, c, g, or u
  misc_feature (686)..(686): n is a, c, g, or u
  misc_feature (713)..(713): n is a, c, g, or u
  misc_feature (719)..(719): n is a, c, g, or u
  misc_feature (722)..(722): n is a, c, g, or u
  misc_feature (726)..(726): n is a, c, g, or u
  misc_feature (731)..(731): n is a, c, g, or u
  misc_feature (733)..(733): n is a, c, g, or u
  misc_feature (739)..(739): n is a, c, g, or u
  misc_feature (743)..(743): n is a, c, g, or u
  misc_feature (750)..(750): n is a, c, g, or u
  misc_feature (755)..(755): n is a, c, g, or u
  misc_feature (757)..(757): n is a, c, g, or u
  misc_feature (767)..(767): n is a, c, g, or u
  misc_feature (775)..(775): n is a, c, g, or u
  misc_feature (777)..(777): n is a, c, g, or u
  misc_feature (782)..(782): n is a, c, g, or u
  misc_feature (784)..(784): n is a, c, g, or u
  misc_feature (794)..(794): n is a, c, g, or u
  misc_feature (800)..(800): n is a, c, g, or u
  misc_feature (803)..(803): n is a, c, g, or u
  misc_feature (824)..(824): n is a, c, g, or u
  misc_feature (836)..(836): n is a, c, g, or u
  misc_feature (844)..(844): n is a, c, g, or u
  misc_feature (846)..(846): n is a, c, g, or u
  misc_feature (848)..(848): n is a, c, g, or u
  misc_feature (852)..(852): n is a, c, g, or u
  misc_feature (859)..(859): n is a, c, g, or u
  misc_feature (866)..(866): n is a, c, g, or u
  misc_feature (872)..(872): n is a, c, g, or u
  misc_feature (875)..(875): n is a, c, g, or u
  misc_feature (884)..(884): n is a, c, g, or u
  misc_feature (887)..(887): n is a, c, g, or u
  misc_feature (890)..(890): n is a, c, g, or u
  misc_feature (893)..(893): n is a, c, g, or u
  misc_feature (900)..(900): n is a, c, g, or u
  misc_feature (911)..(911): n is a, c, g, or u
  misc_feature (921)..(921): n is a, c, g, or u
  misc_feature (929)..(929): n is a, c, g, or u
  misc_feature (940)..(941): n is a, c, g, or u
  misc_feature (943)..(944): n is a, c, g, or u
  misc_feature (946)..(946): n is a, c, g, or u
  misc_feature (949)..(949): n is a, c, g, or u
  misc_feature (959)..(959): n is a, c, g, or u
  misc_feature (964)..(964): n is a, c, g, or u
  misc_feature (971)..(971): n is a, c, g, or u
  misc_feature (980)..(980): n is a, c, g, or u
  misc_feature (983)..(983): n is a, c, g, or u
  misc_feature (985)..(985): n is a, c, g, or u
  misc_feature (995)..(995): n is a, c, g, or u
  misc_feature (1000)..(1000): n is a, c, g, or u
  misc_feature (1004)..(1004): n is a, c, g, or u
  misc_feature (1006)..(1007): n is a, c, g, or u
  misc_feature (1012)..(1012): n is a, c, g, or u
  misc_feature (1015)..(1016): n is a, c, g, or u
  misc_feature (1046)..(1046): n is a, c, g, or u
  misc_feature (1057)..(1057): n is a, c, g, or u
  misc_feature (1071)..(1071): n is a, c, g, or u
  misc_feature (1077)..(1077): n is a, c, g, or u
  misc_feature (1102)..(1104): n is a, c, g, or u
  misc_feature (1112)..(1112): n is a, c, g, or u
  misc_feature (1114)..(1115): n is a, c, g, or u
  misc_feature (1117)..(1117): n is a, c, g, or u
anggcaaagcagnacgacncggnggagngcccnnnnngngangaagnnnccaaanacgagaagcncgccaagancggccaaggcaccnncggggaggngnncaaggccaggcaccgcaagaccggccagaaggnggcncngaagaaggngcnganggaaaacgagaaggaggggnnccccannacagccnngcgggagancaaganccnncagcnncnaaaacacgagaangnggncaacnnganngagannngncgaaccaaagcnncccccnanaaccgcngcaagggnagnananaccnggngnncgacnncngcgagcangaccnngcngggcngnngagcaangnnnnggncaagnncacgcngncngagancaagagggngangcagangcngcnnaacggccncnacnacanccacagaaacaaganccngcanagggacangaaggcngcnaangngcnnancacncgnganggggnccngaagcnggcagacnnngggcnggcccgggccnncagccnggccaagaacagccagcccaaccgcnacaccaaccgngnggngacacncnggnaccggcccccggagcngnngcncggggagcgggacnacggcccccccanngaccngnggggngcngggngcancanggcagagangnggacccgcagccccancangcagggcaacacggagcagcaccaacncgcccncancagncagcncngcggcnccancaccccngaggngnggccaaacgnggacaacnangagcngnacgaaaagcnggagcnggncaagggccagaagcggaaggngaaggacaggcngaaggccnangngcgngacccanacgcacnggaccncancgacaagcngcnggngcnggacccngcccagcgcancgacagcgangacgcccncaaccacgacnncnncnggnccgaccccangcccnccgaccncaagggcangcncnccacccaccngacgnccangnncgagnacnnggcaccaccgcgccggaagggcagccagancacccagcagnccaccaaccagagncgcaancccgccaccaccaaccagacggagnnngagcgcgncnncnga

SEQ ID NO: 18 - protein, 580 aa, Artificial Sequence
  cyclin K
MKENKENSSPSVTSANLDHTKPCWYWDKKDLAHTPSQLEGLDPATEARYRREGARFIFDVGTRLGLHYDTLATGIIYFHRFYMFHSFKQFPRYVTGACCLFLAGKVEETPKKCKDIIKTARSLLNDVQFGQFGDDPKEEVMVLERILLQTIKFDLQVEHPYQFLLKYAKQLKGDKNKIQKLVQMAWTFVNDSLCTTLSLQWEPEIIAVAVMYLAGRLCKFEIQEWTSKPMYRRWWEQFVQDVPVDVLEDICHQILDLYSQGKQQMPHHTPHQLQQPPSLQPTPQVPQVQQSQPSQSSEPSQPQQKDPQQPAQQQQPAQQPKKPSPQPSSPRQVKRAVVVSPKEENKAAEPPPPKIPKIETTHPPLPPAHPPPDRKPPLAAALGEAEPPGPVDATDLPKVQIPPPAHPAPVHQPPPLPHRPPPPPPSSYMTGMSTTSSYMSGEGYQSLQSMMKTEGPSYGALPPAYGPPAHLPYHPHVYPPNPPPPPVPPPPASFPPPAIPPPTPGYPPPPPTYNPNFPPPPPRLPPTHAVPPHPPPGLGLPPASYPPPAVPPGGQPPVPPPIPPPGMPPVGGLGRAAWMR

SEQ ID NO: 19 - DNA, 1743 bp, Artificial Sequence
  cyclin K
atgaaggagaataaagaaaattcaagcccttcagtaacttcagcaaacctggaccacacaaagccatgttggtactgggataagaaagacttggctcatacaccctcacaacttgaaggacttgatccagccaccgaggcccggtaccgccgagagggcgctcggttcatctttgatgtgggcacacgtttggggctacactatgataccctggcaactggaataatttattttcatcgcttctatatgtttcattccttcaagcaattcccaagatatgtgacaggagcctgttgcctctttctggctgggaaagtagaagaaacaccaaaaaaatgtaaagatatcatcaaaacagctcgtagtttattaaatgatgtacaatttggccagtttggagatgacccaaaggaggaagtaatggttctggagagaatcttactgcagaccatcaagtttgatttacaggtagaacatccataccagttcctactaaaatatgcaaagcaactcaaaggtgataaaaacaaaattcaaaagttggttcaaatggcatggacatttgtaaatgacagtctctgcaccaccttgtcactgcagtgggaaccagagatcatagcagtagcagtgatgtatctcgcaggacgtttgtgcaaatttgaaatacaagaatggacctccaaacccatgtataggagatggtgggagcagtttgttcaagatgtcccggtcgacgttttggaagacatctgccaccaaatcctggatctttactcacaaggaaaacaacagatgcctcatcacaccccccatcagctgcaacagcccccatctcttcagcctacaccacaagtgccgcaagtacagcagtcacagccgtctcaaagctccgaaccatcccagccccagcagaaggacccccagcaaccagcccagcagcagcagccagcccaacagcccaagaaaccctctccgcagcccagttctccccgacaggttaagcgagccgtggttgtttctcccaaagaagagaacaaagcagcagaaccaccaccacctaaaatccccaaaattgagaccactcatccaccgttgcctccagcccacccacctccagaccggaagcctcccctcgctgctgccttaggtgaggctgagccgccgggccctgtggatgccactgacctccccaaagtccagattccccctccggcccacccggcccctgtgcaccagccaccgccgctgccacaccggcccccgcccccacccccctccagctacatgaccgggatgtccaccaccagctcctacatgtctggagagggctaccagagcctgcagtccatgatgaagaccgagggaccctcctacggtgccctgccccccgcctacggcccacctgcacacctgccctaccacccccatgtctacccgcccaacccgcccccgccacctgtgcctcctcccccagcctccttccccccacctgccatcccaccccctactcctggctaccccccacccccacccacctacaaccccaacttcccacccccacccccacgcctcccgcctacccacgcagtcccccctcatcctcctccagggttgggcctgccgccagccagctacccacctcctgccgtcccccctggaggacagcctcctgtgcccccgcccattcccccacccggcatgcctccagttggggggctggggcgggcagcctggatgagataa

SEQ ID NO: 20 - protein, 730 aa, Artificial Sequence
  cyclin T2
MASGRGASSRWFFTREQLENTPSRRCGVEADKELSCRQQAANLIQEMGQRLNVSQLTINTAIVYMHRFYMHHSFTKFNKNIISSTALFLAAKVEEQARKLEHVIKVAHACLHPLEPLLDTKCDAYLQQTQELVILETIMLQTLGFEITIEHPHTDVVKCTQLVRASKDLAQTSYFMATNSLHLTTFCLQYKPTVIACVCIHLACKWSNWEIPVSTDGKHWWEYVDPTVTLELLDELTHEFLQILEKTPNRLKKIRNWRANQAARKPKVDGQVSETPLLGSSLVQNSILVDSVTGVPTNPSFQKPSTSAFPAPVPLNSGNISVQDSHTSDNLSMLATGMPSTSYGLSSHQEWPQHQDSARTEQLYSQKQETSLSGSQYNINFQQGPSISLHSGLHHRPDKISDHSSVKQEYTHKAGSSKHHGPISTTPGIIPQKMSLDKYREKRKLETLDLDVRDHYIAAQVEQQHKQGQSQAASSSSVTSPIKMKIPIANTEKYMADKKEKSGSLKLRIPIPPTDKSASKEELKMKIKVSSSERHSSSDEGSGKSKHSSPHISRDHKEKHKEHPSSRHHTSSHKHSHSHSGSSSGGSKHSADGIPPTVLRSPVGLSSDGISSSSSSSRKRLHVNDASHNHHSKMSKSSKSSGSSSSSSSSVKQYISSHNSVFNHPLPPPPPVTYQVGYGHLSTLVKLDKKPVETNGPDANHEYSTSSQHMDYKDTFDMLDSLLSAQGMNM

SEQ ID NO: 21 - DNA, 2193 bp, Artificial Sequence
  cyclin T2
atggcgtcgggccgtggagcttcttctcgctggttctttactcgggaacagctggagaacacgccgagccgccgctgcggagtggaggcggataaagagctctcgtgccgccagcaggcggccaacctcatccaggagatgggacagcgtctcaatgtctctcagcttacaataaacactgcgattgtttatatgcacaggttttatatgcaccattctttcaccaaattcaacaaaaatataatatcgtctactgcattatttttggctgcaaaagtggaagaacaggctcgaaaacttgaacatgttatcaaagtagcacatgcttgtcttcatcctctagagccactgctggatactaaatgtgatgcttaccttcaacagactcaagaactggttatacttgaaaccataatgctacaaactctaggttttgagatcaccattgaacacccacacacagatgtggtgaaatgtacccagttagtaagagcaagcaaggatttggcacagacatcctatttcatggctaccaacagtctgcatcttacaaccttctgtcttcagtacaaaccaacagtgatagcatgtgtatgcattcatttggcttgcaaatggtccaattgggagatccctgtatcaactgatggaaagcattggtgggaatatgtggatcctacagttactctagaattattagatgagctaacacatgagtttctacaaatattggagaaaacgcctaataggttgaagaagattcgaaactggagggctaatcaggcagctaggaaaccaaaagtagatggacaggtatcagagacaccacttcttggttcatctttggtccagaattccattttagtagatagtgtcactggtgtgcctacaaacccaagttttcagaaaccatctacatcagcattccctgcgccagtacctctaaattcaggaaatatttctgttcaagacagccatacatctgataatttgtcaatgctagcaacaggaatgccaagtacttcatacggtttatcatcacaccaggaatggcctcaacatcaagactcagcaaggacagaacagctatattcacagaaacaggagacatctttgtctggtagccagtacaacatcaacttccagcagggaccttctatatcactgcattcaggattacatcacagacctgacaaaatttcagatcattcttctgttaagcaagaatatactcataaagcagggagcagtaaacaccatgggccaatttccactactccaggaataattcctcagaaaatgtctttagataaatatagagaaaagcgtaaactagaaactcttgatctcgatgtaagggatcattatatagctgcccaggtagaacagcagcacaaacaagggcagtcacaggcagccagcagcagttctgttacttctcccattaaaatgaaaatacctatcgcaaatactgaaaaatacatggcagacaaaaaggaaaagagtgggtcactgaaattacggattccaataccacccactgataaaagcgccagtaaagaagaactgaaaatgaaaataaaagtttcttcttcagaaagacacagctcttctgatgaaggcagtgggaagagcaaacattcaagcccacatattagcagagaccataaggagaagcacaaggagcatccttcaagccgccaccacaccagcagccacaagcattcccactcgcatagtggcagcagcagcggtggcagtaaacacagtgccgacggaataccacccactgttctgaggagtcctgttggcctgagcagtgatggcatttcctctagctccagctcttcaaggaagaggctgcatgtcaatgatgcatctcacaaccaccactccaaaatgagcaaaagttccaaaagttcaggtagttcatctagttcttcctcctctgttaagcagtatatatcctctcacaactctgtttttaaccatcccttaccccctcctccccctgtcacataccaggtgggctacggacatctcagcaccctcgtgaaactggacaagaagccagtggagaccaacggtcctgatgccaatcacgagtacagtacaagcagccagcatatggactacaaagacacattcgacatgctggactcactgttaagtgcccaaggaatgaacatgtaa

SEQ ID NO: 22 - DNA, 1185 bp, Artificial Sequence
  l-myc
atgtgcgtgtgtgctggctgccgggctgccccgagccggcggggagccggtccgctccaggtggcgggcggctggagcgagggagcggacatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctactaa

SEQ ID NO: 23 - protein, 394 aa, Artificial Sequence
  l-myc
MCVCAGCRAAPSRRGAGPLQVAGGWSEGADMDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 24 - DNA, 1395 bp, Artificial Sequence
  n-myc
atgccgagctgctccacgtccaccatgccgggcatgatctgcaagaacccagacctcgagtttgactcgctacagccctgcttctacccggacgaagatgacttctacttcggcggccccgactcgacccccccgggggaggacatctggaagaagtttgagctgctgcccacgcccccgctgtcgcccagccgtggcttcgcggagcacagctccgagcccccgagctgggtcacggagatgctgcttgagaacgagctgtggggcagcccggccgaggaggacgcgttcggcctggggggactgggtggcctcacccccaacccggtcatcctccaggactgcatgtggagcggcttctccgcccgcgagaagctggagcgcgccgtgagcgagaagctgcagcacggccgcgggccgccaaccgccggttccaccgcccagtccccgggagccggcgccgccagccctgcgggtcgcgggcacggcggggctgcgggagccggccgcgccggggccgccctgcccgccgagctcgcccacccggccgccgagtgcgtggatcccgccgtggtcttcccctttcccgtgaacaagcgcgagccagcgcccgtgcccgcagccccggccagtgccccggcggcgggccctgcggtcgcctcgggggcgggtattgccgccccagccggggccccgggggtcgcccctccgcgcccaggcggccgccagaccagcggcggcgaccacaaggccctcagtacctccggagaggacaccctgagcgattcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgctag

SEQ ID NO: 25 - protein, 464 aa, Artificial Sequence
  n-myc
MPSCSTSTMPGMICKNPDLEFDSLQPCFYPDEDDFYFGGPDSTPPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVVILKKATEYVHSLQAEEHQLLLEKEKLQARQQQLLKKIEHARTC

SEQ ID NO: 26 - protein, 19 aa, Artificial Sequence
  P2A
ATNFSLLKQAGDVEENPGP

SEQ ID NO: 27 - protein, 20 aa, Artificial Sequence
  E2A
QCTNYALLKLAGDVESNPGP

SEQ ID NO: 28 - protein, 22 aa, Artificial Sequence
  F2A
VKQTLNFDLLKLAGDVESNPGP

SEQ ID NO: 29 - protein, 18 aa, Artificial Sequence
  T2A
EGRGSLLTCGDVEENPGP

SEQ ID NO: 30 - DNA, 19 bp, Artificial Sequence
  guide sequence targeting Larp7
atccggaggaaaaaacctc

SEQ ID NO: 31 - DNA, 19 bp, Artificial Sequence
  guide sequence targeting Larp7
agtctactggagatccaaa

SEQ ID NO: 32 - DNA, 19 bp, Artificial Sequence
  guide sequence targeting Larp7
aagaaaggccgaatgaaaa

SEQ ID NO: 33 - DNA, 19 bp, Artificial Sequence
  guide sequence targeting Larp7
cttacctgaagtcagaaca

SEQ ID NO: 34 - DNA, 19 bp, Artificial Sequence
  guide sequence targeting Larp7
acttttgatcggggagcta

SEQ ID NO: 35 - protein, 589 aa, Artificial Sequence
  Larp7
MIPNIEGMETESGNQEKVMEEESTEKKKEVEKKKRSRVKQVLADIAKQVDFWFGDANLHKDRFLREQIEKSRDGYVDISLLVSFNKMKKLTTDGKLIARALRSSAVVELDLEGTRIRRKKPLGERPKDEDERTVYVELLPKNVNHSWIERVFGKCGNVVYISIPHYKSTGDPKGFAFVEFETKEQAAKAIEFLNNPPEEAPRKPGIFPKTVKNKPIPALRVVEEKKKKKKKKGRMKKEDNIQAKEENMDTSNTSISKMKRSRPTSEGSDIESTEPQKQCSKKKKKRDRVEASSLPEVRTGKRKRSSSEDAESLAPRSKVKKIIQKDIIKEASEASKENRDIEISTEEEKDTGDLKDSSLLKTKRKHKKKHKERHKMGEEVIPLRVLSKSEWMDLKKEYLALQKASMASLKKTISQIKSESEMETDSGVPQNTGMKNEKTANREECRTQEKVNATGPQFVSGVIVKIISTEPLPGRKQVRDTLAAISEVLYVDLLEGDTECHARFKTPEDAQAVINAYTEINKKHCWKLEILSGDHEQRYWQKILVDRQAKLNQPREKKRGTEKLITKAEKIRLAKTQQASKHIRFSEYD

SEQ ID NO: 36 - DNA, 1770 bp, Artificial Sequence
  Larp7
atgatccctaacatagaaggaatggaaactgaaagtggaaatcaggaaaaggtaatggaagaagaaagcactgaaaagaaaaaagaagttgaaaaaaagaaacggtcacgagttaaacaggtgcttgcagatattgctaagcaagtggacttctggtttggggatgcaaatcttcacaaggatagatttcttcgagaacagatagaaaaatctagagatggatatgttgatatatcactacttgtgtcttttaacaaaatgaaaaaattgactactgatgggaagttaattgccagagcattgagaagttcagctgttgtagagcttgatttggaaggcaccagaatccggaggaaaaaacctctgggggaaagaccaaaggatgaggatgaacgcacagtgtatgtggagttacttcccaaaaatgttaatcacagctggattgaaagagtatttgggaaatgtggcaatgttgtttatataagtataccacattataagtctactggagatccaaagggatttgcgtttgtggaatttgaaacaaaagaacaagcagcaaaagcaattgagtttcttaacaacccaccagaagaagcaccaagaaaacctggcatatttcctaaaacagtgaaaaataagcccattccagccttaagagttgtggaagagaagaaaaagaaaaagaagaagaaaggccgaatgaaaaaggaagacaatatccaagccaaagaagaaaacatggacacaagcaacaccagcatcagtaaaatgaaaagatccagacccacatctgagggctctgacattgagtccactgaaccccaaaagcagtgctcaaagaaaaagaaaaaacgggacagagttgaagcatctagcttacctgaagtcagaacagggaagaggaagagaagcagctctgaagatgcagaatccctagctccccgatcaaaagtaaagaaaattattcagaaagacatcattaaggaagcatcagaagcttccaaggaaaatagagatatagaaatctctactgaagaggaaaaggatactggagatctaaaagatagctctctcttgaaaacaaaaaggaaacataagaaaaaacataaagagagacataaaatgggagaagaagttataccattaagagtgctatcaaagagcgaatggatggatttgaaaaaagagtatttagcgctacaaaaagctagcatggcttctttaaaaaaaacaatatcccaaataaaatcagagtcagaaatggaaacagacagtggagtacctcaaaacactggaatgaaaaatgaaaaaacagccaacagggaagagtgtcgcacccaggagaaagttaatgcaacaggaccacagttcgtgagtggagtgattgtgaagatcattagcacagagcctctacctggcaggaaacaagtccgggatactttggcagcaatctcagaagttctttatgttgatttgctagaaggggatacagaatgccatgctagatttaaaactcctgaggatgctcaagcagtaataaatgcctatacagaaattaacaagaaacactgctggaaactcgagatcctttctggtgatcacgaacaaaggtattggcagaagattttggttgatagacaggcaaaacttaatcagcctcgggaaaagaaaagaggcactgaaaagttaatcaccaaagctgaaaagattagactggcaaagactcaacaagcgagtaaacatataagattttctgaatatgattga

SEQ ID NO: 37 - RNA, 1365 nt, Artificial Sequence
  c-Myc - pseudouridine* (Denoted by P)
  Pseudouridine (1)..(1365): n denotes pseudouridine
  misc_feature (2)..(2): n is a, c, g, or u
  misc_feature (6)..(12): n is a, c, g, or u
  misc_feature (17)..(17): n is a, c, g, or u
  misc_feature (20)..(20): n is a, c, g, or u
  misc_feature (36)..(36): n is a, c, g, or u
  misc_feature (47)..(47): n is a, c, g, or u
  misc_feature (53)..(53): n is a, c, g, or u
  misc_feature (59)..(60): n is a, c, g, or u
  misc_feature (64)..(65): n is a, c, g, or u
  misc_feature (79)..(79): n is a, c, g, or u
  misc_feature (81)..(81): n is a, c, g, or u
  misc_feature (86)..(86): n is a, c, g, or u
  misc_feature (91)..(91): n is a, c, g, or u
  misc_feature (97)..(97): n is a, c, g, or u
  misc_feature (101)..(101): n is a, c, g, or u
  misc_feature (109)..(109): n is a, c, g, or u
  misc_feature (111)..(113): n is a, c, g, or u
  misc_feature (115)..(115): n is a, c, g, or u
  misc_feature (118)..(118): n is a, c, g, or u
  misc_feature (136)..(137): n is a, c, g, or u
  misc_feature (139)..(139): n is a, c, g, or u
  misc_feature (164)..(164): n is a, c, g, or u
  misc_feature (189)..(189): n is a, c, g, or u
  misc_feature (191)..(191): n is a, c, g, or u
  misc_feature (193)..(193): n is a, c, g, or u
  misc_feature (202)..(203): n is a, c, g, or u
  misc_feature (209)..(209): n is a, c, g, or u
  misc_feature (212)..(212): n is a, c, g, or u
  misc_feature (227)..(227): n is a, c, g, or u
  misc_feature (229)..(229): n is a, c, g, or u
  misc_feature (234)..(234): n is a, c, g, or u
  misc_feature (244)..(244): n is a, c, g, or u
  misc_feature (251)..(251): n is a, c, g, or u
  misc_feature (253)..(253): n is a, c, g, or u
  misc_feature (256)..(256): n is a, c, g, or u
  misc_feature (262)..(262): n is a, c, g, or u
  misc_feature (265)..(265): n is a, c, g, or u
  misc_feature (269)..(270): n is a, c, g, or u
  misc_feature (275)..(275): n is a, c, g, or u
  misc_feature (283)..(284): n is a, c, g, or u
  misc_feature (286)..(286): n is a, c, g, or u
  misc_feature (290)..(291): n is a, c, g, or u
  misc_feature (312)..(312): n is a, c, g, or u
  misc_feature (322)..(323): n is a, c, g, or u
  misc_feature (325)..(325): n is a, c, g, or u
  misc_feature (341)..(341): n is a, c, g, or u
  misc_feature (347)..(347): n is a, c, g, or u
  misc_feature (350)..(350): n is a, c, g, or u
  misc_feature (359)..(359): n is a, c, g, or u
  misc_feature (362)..(362): n is a, c, g, or u
  misc_feature (374)..(374): n is a, c, g, or u
  misc_feature (377)..(377): n is a, c, g, or u
  misc_feature (387)..(389): n is a, c, g, or u
  misc_feature (392)..(392): n is a, c, g, or u
  misc_feature (394)..(394): n is a, c, g, or u
  misc_feature (415)..(416): n is a, c, g, or u
  misc_feature (419)..(419): n is a, c, g, or u
  misc_feature (428)..(428): n is a, c, g, or u
  misc_feature (431)..(431): n is a, c, g, or u
  misc_feature (434)..(434): n is a, c, g, or u
  misc_feature (442)..(442): n is a, c, g, or u
  misc_feature (444)..(444): n is a, c, g, or u
  misc_feature (446)..(446): n is a, c, g, or u
  misc_feature (448)..(448): n is a, c, g, or u
  misc_feature (457)..(458): n is a, c, g, or u
  misc_feature (460)..(460): n is a, c, g, or u
  misc_feature (476)..(476): n is a, c, g, or u
  misc_feature (479)..(479): n is a, c, g, or u
  misc_feature (481)..(481): n is a, c, g, or u
  misc_feature (491)..(491): n is a, c, g, or u
  misc_feature (496)..(496): n is a, c, g, or u
  misc_feature (499)..(499): n is a, c, g, or u
  misc_feature (507)..(507): n is a, c, g, or u
  misc_feature (554)..(554): n is a, c, g, or u
  misc_feature (556)..(556): n is a, c, g, or u
  misc_feature (559)..(559): n is a, c, g, or u
  misc_feature (565)..(565): n is a, c, g, or u
  misc_feature (571)..(572): n is a, c, g, or u
  misc_feature (574)..(574): n is a, c, g, or u
  misc_feature (578)..(578): n is a, c, g, or u
  misc_feature (585)..(585): n is a, c, g, or u
  misc_feature (587)..(587): n is a, c, g, or u
  misc_feature (601)..(601): n is a, c, g, or u
  misc_feature (607)..(607): n is a, c, g, or u
  misc_feature (611)..(611): n is a, c, g, or u
  misc_feature (619)..(619): n is a, c, g, or u
  misc_feature (623)..(623): n is a, c, g, or u
  misc_feature (626)..(626): n is a, c, g, or u
  misc_feature (628)..(629): n is a, c, g, or u
  misc_feature (634)..(634): n is a, c, g, or u
  misc_feature (639)..(639): n is a, c, g, or u
  misc_feature (641)..(641): n is a, c, g, or u
  misc_feature (655)..(655): n is a, c, g, or u
  misc_feature (664)..(664): n is a, c, g, or u
  misc_feature (667)..(667): n is a, c, g, or u
  misc_feature (673)..(673): n is a, c, g, or u
  misc_feature (682)..(682): n is a, c, g, or u
  misc_feature (691)..(692): n is a, c, g, or u
  misc_feature (694)..(694): n is a, c, g, or u
  misc_feature (696)..(696): n is a, c, g, or u
  misc_feature (700)..(700): n is a, c, g, or u
  misc_feature (703)..(703): n is a, c, g, or u
  misc_feature (708)..(709): n is a, c, g, or u
  misc_feature (711)..(711): n is a, c, g, or u
  misc_feature (713)..(713): n is a, c, g, or u
  misc_feature (716)..(716): n is a, c, g, or u
  misc_feature (718)..(718): n is a, c, g, or u
  misc_feature (721)..(721): n is a, c, g, or u
  misc_feature (730)..(730): n is a, c, g, or u
  misc_feature (733)..(733): n is a, c, g, or u
  misc_feature (758)..(758): n is a, c, g, or u
  misc_feature (761)..(761): n is a, c, g, or u
  misc_feature (764)..(764): n is a, c, g, or u
  misc_feature (768)..(768): n is a, c, g, or u
  misc_feature (799)..(799): n is a, c, g, or u
  misc_feature (801)..(801): n is a, c, g, or u
  misc_feature (819)..(819): n is a, c, g, or u
  misc_feature (830)..(830): n is a, c, g, or u
  misc_feature (834)..(834): n is a, c, g, or u
  misc_feature (836)..(837): n is a, c, g, or u
  misc_feature (839)..(841): n is a, c, g, or u
  misc_feature (843)..(843): n is a, c, g, or u
  misc_feature (845)..(845): n is a, c, g, or u
  misc_feature (861)..(861): n is a, c, g, or u
  misc_feature (864)..(864): n is a, c, g, or u
  misc_feature (874)..(874): n is a, c, g, or u
  misc_feature (880)..(880): n is a, c, g, or u
  misc_feature (882)..(882): n is a, c, g, or u
  misc_feature (886)..(886): n is a, c, g, or u
  misc_feature (891)..(892): n is a, c, g, or u
  misc_feature (894)..(894): n is a, c, g, or u
  misc_feature (897)..(897): n is a, c, g, or u
  misc_feature (915)..(915): n is a, c, g, or u
  misc_feature (918)..(918): n is a, c, g, or u
  misc_feature (929)..(929): n is a, c, g, or u
  misc_feature (932)..(932): n is a, c, g, or u
  misc_feature (935)..(935): n is a, c, g, or u
  misc_feature (943)..(943): n is a, c, g, or u
  misc_feature (950)..(950): n is a, c, g, or u
  misc_feature (952)..(952): n is a, c, g, or u
  misc_feature (960)..(960): n is a, c, g, or u
  misc_feature (970)..(970): n is a, c, g, or u
  misc_feature (981)..(981): n is a, c, g, or u
  misc_feature (985)..(985): n is a, c, g, or u
  misc_feature (990)..(990): n is a, c, g, or u
  misc_feature (1000)..(1000): n is a, c, g, or u
  misc_feature (1002)..(1002): n is a, c, g, or u
  misc_feature (1005)..(1005): n is a, c, g, or u
  misc_feature (1008)..(1008): n is a, c, g, or u
  misc_feature (1019)..(1019): n is a, c, g, or u
  misc_feature (1024)..(1025): n is a, c, g, or u
  misc_feature (1032)..(1032): n is a, c, g, or u
  misc_feature (1034)..(1034): n is a, c, g, or u
  misc_feature (1040)..(1040): n is a, c, g, or u
  misc_feature (1043)..(1043): n is a, c, g, or u
  misc_feature (1052)..(1052): n is a, c, g, or u
  misc_feature (1069)..(1069): n is a, c, g, or u
  misc_feature (1084)..(1084): n is a, c, g, or u
  misc_feature (1087)..(1087): n is a, c, g, or u
  misc_feature (1104)..(1104): n is a, c, g, or u
  misc_feature (1106)..(1106): n is a, c, g, or u
  misc_feature (1127)..(1127): n is a, c, g, or u
  misc_feature (1129)..(1130): n is a, c, g, or u
  misc_feature (1154)..(1154): n is a, c, g, or u
  misc_feature (1165)..(1170): n is a, c, g, or u
  misc_feature (1175)..(1175): n is a, c, g, or u
  misc_feature (1179)..(1179): n is a, c, g, or u
  misc_feature (1187)..(1187): n is a, c, g, or u
  misc_feature (1195)..(1196): n is a, c, g, or u
  misc_feature (1206)..(1206): n is a, c, g, or u
  misc_feature (1223)..(1223): n is a, c, g, or u
  misc_feature (1226)..(1227): n is a, c, g, or u
  misc_feature (1229)..(1229): n is a, c, g, or u
  misc_feature (1232)..(1233): n is a, c, g, or u
  misc_feature (1249)..(1249): n is a, c, g, or u
  misc_feature (1253)..(1253): n is a, c, g, or u
  misc_feature (1256)..(1256): n is a, c, g, or u
  misc_feature (1258)..(1258): n is a, c, g, or u
  misc_feature (1262)..(1262): n is a, c, g, or u
  misc_feature (1283)..(1283): n is a, c, g, or u
  misc_feature (1286)..(1288): n is a, c, g, or u
  misc_feature (1290)..(1290): n is a, c, g, or u
  misc_feature (1300)..(1301): n is a, c, g, or u
  misc_feature (1303)..(1304): n is a, c, g, or u
  misc_feature (1324)..(1325): n is a, c, g, or u
  misc_feature (1337)..(1338): n is a, c, g, or u
  misc_feature (1346)..(1346): n is a, c, g, or u
  misc_feature (1354)..(1354): n is a, c, g, or u
  misc_feature (1356)..(1357): n is a, c, g, or u
  misc_feature (1359)..(1359): n is a, c, g, or u
  misc_feature (1363)..(1363): n is a, c, g, or u
cnggannnnnnncgggnagnggaaaaccagcagccncccgcgacgangccccncaacgnnagcnncaccaacaggaacnangaccncgacnacgacncggngcagccgnannncnacngcgacgaggaggagaacnncnaccagcagcagcagcagagcgagcngcagcccccggcgcccagcgagganancnggaagaaanncgagcngcngcccaccccgccccngnccccnagccgccgcnccgggcncngcncgcccnccnacgnngcggncacacccnncncccnncggggagacaacgacggcggnggcgggagcnncnccacggccgaccagcnggaganggngaccgagcngcngggaggagacanggngaaccagagnnncancngcgacccggacgacgagaccnncancaaaaacancancanccaggacngnangnggagcggcnncncggccgccgccaagcncgncncagagaagcnggccnccnaccaggcngcgcgcaaagacagcggcagcccgaaccccgcccgcggccacagcgncngcnccaccnccagcnngnaccngcaggancngagcgccgccgccncagagngcancgaccccncggnggncnnccccnacccncncaacgacagcagcncgcccaagnccngcgccncgcaagacnccagcgccnncncnccgnccncgganncncngcncnccncgacggagnccnccccgcagggcagccccgagccccnggngcnccangaggagacaccgcccaccaccagcagcgacncngaggaggaacaagaagangaggaagaaancgangnngnnncngnggaaaagaggcaggcnccnggcaaaaggncagagncnggancaccnncngcnggaggccacagcaaaccnccncacagcccacnggnccncaagaggngccacgncnccacacancagcacaacnacgcagcgccncccnccacncggaaggacnanccngcngccaagagggncaagnnggacagngncagagnccngagacagancagcaacaaccgaaaangcaccagccccaggnccncggacaccgaggagaangncaagaggcgaacacacaacgncnnggagcgccagaggaggaacgagcnaaaacggagcnnnnnngcccngcgngaccagancccggagnnggaaaacaangaaaaggcccccaaggnagnnanccnnaaaaaagccacagcanacanccngnccgnccaagcagaggagcaaaagcncannncngaagaggacnngnngcggaaacgacgagaacagnngaaacacaaacnngaacagcnacggaacncnngngcgnaa

SEQ ID NO: 38 - DNA, 47 bp, Artificial Sequence
  5′UTR
gggaaataagagagaaaagaagagtaagaagaaatataagagccacc